SEQUENCE LISTING (39 sequences)

SEQ ID NO: 1 - protein, 707 aa, Unknown
  Derived from a clade of megaphages containing Cas Phi
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 2 - protein, 757 aa, Unknown
  Derived from a clade of megaphages containing Cas Phi
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 3 - protein, 766 aa, Unknown
  Derived from a clade of megaphages containing Cas Phi
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 4 - protein, 765 aa, Unknown
  Derived from a clade of megaphages containing Cas Phi
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 5 - protein, 793 aa, Unknown
  Derived from a clade of megaphages containing Cas Phi
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 6 - protein, 441 aa, Unknown
  Derived from a clade of megaphages containing Cas Phi
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 7 - protein, 772 aa, Unknown
  Derived from a clade of megaphages containing Cas Phi
MSNTAVSTREHMSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 8 - protein, 717 aa, Unknown
  Derived from a clade of megaphages containing Cas Phi
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 9 - protein, 793 aa, Unknown
  Derived from a clade of megaphages containing Cas Phi
MRSSREIGDKILMRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 10 - protein, 812 aa, Unknown
  Derived from a clade of megaphages containing Cas Phi
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 11 - DNA, 2121 bp, Artificial
  Bacteria optimized
  misc_feature (1)..(2121): cas_phi-1
atggcggataccccgaccctgtttacccagtttctgcgccatcatctgccgggccagcgctttcgcaaagatattctgaaacaggcgggccgcattctggcgaacaaaggcgaagatgcgaccattgcgtttctgcgcggcaaaagcgaagaaagcccgccggattttcagccgccggtgaaatgcccgattattgcgtgcagccgcccgctgaccgaatggccgatttatcaggcgagcgtggcgattcagggctatgtgtatggccagagcctggcggaatttgaagcgagcgatccgggctgcagcaaagatggcctgctgggctggtttgataaaaccggcgtgtgcaccgattattttagcgtgcagggcctgaacctgatttttcagaacgcgcgcaaacgctatattggcgtgcagaccaaagtgaccaaccgcaacgaaaaacgccataaaaaactgaaacgcattaacgcgaaacgcattgcggaaggcctgccggaactgaccagcgatgaaccggaaagcgcgctggatgaaaccggccatctgattgatccgccgggcctgaacaccaacatttattgctatcagcaggtgagcccgaaaccgctggcgctgagcgaagtgaaccagctgccgaccgcgtatgcgggctatagcaccagcggcgatgatccgattcagccgatggtgaccaaagatcgcctgagcattagcaaaggccagccgggctatattccggaacatcagcgcgcgctgctgagccagaaaaaacatcgccgcatgcgcggctatggcctgaaagcgcgcgcgctgctggtgattgtgcgcattcaggatgattgggcggtgattgatctgcgcagcctgctgcgcaacgcgtattggcgccgcattgtgcagaccaaagaaccgagcaccattaccaaactgctgaaactggtgaccggcgatccggtgctggatgcgacccgcatggtggcgacctttacctataaaccgggcattgtgcaggtgcgcagcgcgaaatgcctgaaaaacaaacagggcagcaaactgtttagcgaacgctatctgaacgaaaccgtgagcgtgaccagcattgatctgggcagcaacaacctggtggcggtggcgacctatcgcctggtgaacggcaacaccccggaactgctgcagcgctttaccctgccgagccatctggtgaaagattttgaacgctataaacaggcgcatgataccctggaagatagcattcagaaaaccgcggtggcgagcctgccgcagggccagcagaccgaaattcgcatgtggagcatgtatggctttcgcgaagcgcaggaacgcgtgtgccaggaactgggcctggcggatggcagcattccgtggaacgtgatgaccgcgaccagcaccattctgaccgatctgtttctggcgcgcggcggcgatccgaaaaaatgcatgtttaccagcgaaccgaaaaaaaaaaaaaacagcaaacaggtgctgtataaaattcgcgatcgcgcgtgggcgaaaatgtatcgcaccctgctgagcaaagaaacccgcgaagcgtggaacaaagcgctgtggggcctgaaacgcggcagcccggattatgcgcgcctgagcaaacgcaaagaagaactggcgcgccgctgcgtgaactataccattagcaccgcggaaaaacgcgcgcagtgcggccgcaccattgtggcgctggaagatctgaacattggcttttttcatggccgcggcaaacaggaaccgggctgggtgggcctgtttacccgcaaaaaagaaaaccgctggctgatgcaggcgctgcataaagcgtttctggaactggcgcatcatcgcggctatcatgtgattgaagtgaacccggcgtataccagccagacctgcccggtgtgccgccattgcgatccggataaccgcgatcagcataaccgcgaagcgtttcattgcattggctgcggctttcgcggcaacgcggatctggatgtggcgacccataacattgcgatggtggcgattaccggcgaaagcctgaaacgcgcgcgcggcagcgtggcgagcaaaaccccgcagccgctggcggcggaa

SEQ ID NO: 12 - DNA, 2271 bp, Artificial
  Bacteria optimized
  misc_feature (1)..(2271): cas_phi-2
atgccgaaaccggcggtggaaagcgaatttagcaaagtgctgaaaaaacattttccgggcgaacgctttcgcagcagctatatgaaacgcggcggcaaaattctggcggcgcagggcgaagaagcggtggtggcgtatctgcagggcaaaagcgaagaagaaccgccgaactttcagccgccggcgaaatgccatgtggtgaccaaaagccgcgattttgcggaatggccgattatgaaagcgagcgaagcgattcagcgctatatttatgcgctgagcaccaccgaacgcgcggcgtgcaaaccgggcaaaagcagcgaaagccatgcggcgtggtttgcggcgaccggcgtgagcaaccatggctatagccatgtgcagggcctgaacctgatttttgatcataccctgggccgctatgatggcgtgctgaaaaaagtgcagctgcgcaacgaaaaagcgcgcgcgcgcctggaaagcattaacgcgagccgcgcggatgaaggcctgccggaaattaaagcggaagaagaagaagtggcgaccaacgaaaccggccatctgctgcagccgccgggcattaacccgagcttttatgtgtatcagaccattagcccgcaggcgtatcgcccgcgcgatgaaattgtgctgccgccggaatatgcgggctatgtgcgcgatccgaacgcgccgattccgctgggcgtggtgcgcaaccgctgcgatattcagaaaggctgcccgggctatattccggaatggcagcgcgaagcgggcaccgcgattagcccgaaaaccggcaaagcggtgaccgtgccgggcctgagcccgaaaaaaaacaaacgcatgcgccgctattggcgcagcgaaaaagaaaaagcgcaggatgcgctgctggtgaccgtgcgcattggcaccgattgggtggtgattgatgtgcgcggcctgctgcgcaacgcgcgctggcgcaccattgcgccgaaagatattagcctgaacgcgctgctggatctgtttaccggcgatccggtgattgatgtgcgccgcaacattgtgacctttacctataccctggatgcgtgcggcacctatgcgcgcaaatggaccctgaaaggcaaacagaccaaagcgaccctggataaactgaccgcgacccagaccgtggcgctggtggcgattgatctgggccagaccaacccgattagcgcgggcattagccgcgtgacccaggaaaacggcgcgctgcagtgcgaaccgctggatcgctttaccctgccggatgatctgctgaaagatattagcgcgtatcgcattgcgtgggatcgcaacgaagaagaactgcgcgcgcgcagcgtggaagcgctgccggaagcgcagcaggcggaagtgcgcgcgctggatggcgtgagcaaagaaaccgcgcgcacccagctgtgcgcggattttggcctggatccgaaacgcctgccgtgggataaaatgagcagcaacaccacctttattagcgaagcgctgctgagcaacagcgtgagccgcgatcaggtgttttttaccccggcgccgaaaaaaggcgcgaaaaaaaaagcgccggtggaagtgatgcgcaaagatcgcacctgggcgcgcgcgtataaaccgcgcctgagcgtggaagcgcagaaactgaaaaacgaagcgctgtgggcgctgaaacgcaccagcccggaatatctgaaactgagccgccgcaaagaagaactgtgccgccgcagcattaactatgtgattgaaaaaacccgccgccgcacccagtgccagattgtgattccggtgattgaagatctgaacgtgcgcttttttcatggcagcggcaaacgcctgccgggctgggataacttttttaccgcgaaaaaagaaaaccgctggtttattcagggcctgcataaagcgtttagcgatctgcgcacccatcgcagcttttatgtgtttgaagtgcgcccggaacgcaccagcattacctgcccgaaatgcggccattgcgaagtgggcaaccgcgatggcgaagcgtttcagtgcctgagctgcggcaaaacctgcaacgcggatctggatgtggcgacccataacctgacccaggtggcgctgaccggcaaaaccatgccgaaacgcgaagaaccgcgcgatgcgcagggcaccgcgccggcgcgcaaaaccaaaaaagcgagcaaaagcaaagcgccgccggcggaacgcgaagatcagaccccggcgcaggaaccgagccagaccagc

SEQ ID NO: 13 - DNA, 2298 bp, Artificial
  Bacteria optimized
  misc_feature (1)..(2298): cas_phi-3
atggaaaaagaaattaccgaactgaccaaaattcgccgcgaatttccgaacaaaaaatttagcagcaccgatatgaaaaaagcgggcaaactgctgaaagcggaaggcccggatgcggtgcgcgattttctgaacagctgccaggaaattattggcgattttaaaccgccggtgaaaaccaacattgtgagcattagccgcccgtttgaagaatggccggtgagcatggtgggccgcgcgattcaggaatattattttagcctgaccaaagaagaactggaaagcgtgcatccgggcaccagcagcgaagatcataaaagcttttttaacattaccggcctgagcaactataactataccagcgtgcagggcctgaacctgatttttaaaaacgcgaaagcgatttatgatggcaccctggtgaaagcgaacaacaaaaacaaaaaactggaaaaaaaatttaacgaaattaaccataaacgcagcctggaaggcctgccgattattaccccggattttgaagaaccgtttgatgaaaacggccatctgaacaacccgccgggcattaaccgcaacatttatggctatcagggctgcgcggcgaaagtgtttgtgccgagcaaacataaaatggtgagcctgccgaaagaatatgaaggctataaccgcgatccgaacctgagcctggcgggctttcgcaaccgcctggaaattccggaaggcgaaccgggccatgtgccgtggtttcagcgcatggatattccggaaggccagattggccatgtgaacaaaattcagcgctttaactttgtgcatggcaaaaacagcggcaaagtgaaatttagcgataaaaccggccgcgtgaaacgctatcatcatagcaaatataaagatgcgaccaaaccgtataaatttctggaagaaagcaaaaaagtgagcgcgctggatagcattctggcgattattaccattggcgatgattgggtggtgtttgatattcgcggcctgtatcgcaacgtgttttatcgcgaactggcgcagaaaggcctgaccgcggtgcagctgctggatctgtttaccggcgatccggtgattgatccgaaaaaaggcgtggtgacctttagctataaagaaggcgtggtgccggtgtttagccagaaaattgtgccgcgctttaaaagccgcgataccctggaaaaactgaccagccagggcccggtggcgctgctgagcgtggatctgggccagaacgaaccggtggcggcgcgcgtgtgcagcctgaaaaacattaacgataaaattaccctggataacagctgccgcattagctttctggatgattataaaaaacagattaaagattatcgcgatagcctggatgaactggaaattaaaattcgcctggaagcgattaacagcctggaaaccaaccagcaggtggaaattcgcgatctggatgtgtttagcgcggatcgcgcgaaagcgaacaccgtggatatgtttgatattgatccgaacctgattagctgggatagcatgagcgatgcgcgcgtgagcacccagattagcgatctgtatctgaaaaacggcggcgatgaaagccgcgtgtattttgaaattaacaacaaacgcattaaacgcagcgattataacattagccagctggtgcgcccgaaactgagcgatagcacccgcaaaaacctgaacgatagcatttggaaactgaaacgcaccagcgaagaatatctgaaactgagcaaacgcaaactggaactgagccgcgcggtggtgaactataccattcgccagagcaaactgctgagcggcattaacgatattgtgattattctggaagatctggatgtgaaaaaaaaatttaacggccgcggcattcgcgatattggctgggataacttttttagcagccgcaaagaaaaccgctggtttattccggcgtttcataaagcgtttagcgaactgagcagcaaccgcggcctgtgcgtgattgaagtgaacccggcgtggaccagcgcgacctgcccggattgcggcttttgcagcaaagaaaaccgcgatggcattaactttacctgccgcaaatgcggcgtgagctatcatgcggatattgatgtggcgaccctgaacattgcgcgcgtggcggtgctgggcaaaccgatgagcggcccggcggatcgcgaacgcctgggcgataccaaaaaaccgcgcgtggcgcgcagccgcaaaaccatgaaacgcaaagatattagcaacagcaccgtggaagcgatggtgaccgcg

SEQ ID NO: 14 - DNA, 2298 bp, Artificial
  Bacteria optimized
  misc_feature (1)..(2298): cas_phi-4
atgtattcgctggagatggcagatttaaaatcggaaccttcgctgttggcaaagctgctgcgcgaccgcttccctggcaagtactggcttccaaaatattggaaattagcggaaaagaagcgcctgacgggcggggaagaagctgcctgtgaatatatggcagacaaacaattggactcccccccacccaattttcgtccgcctgctcgttgtgtcattttagcaaaatcgcgcccgttcgaggactggcccgtccaccgtgtcgcgtcaaaggcacagagcttcgtaattgggttgtctgaacaggggttcgcggcactgcgtgctgctccaccctcaacagcggacgctcgccgtgactggttacgctcacatggggcgagcgaggacgacttaatggccttggaggctcaacttctggagacaatcatgggaaatgcgatttctcttcacggcggcgtattgaagaaaatcgacaacgcaaatgttaaagctgccaaacgcttaagtggccgtaacgaggcgcgccttaataagggtttgcaagaacttccccccgaacaggagggaagtgcctatggagcagacggtttattagtgaaccccccgggccttaatcttaatatctattgccgcaagtcgtgttgtccaaagcctgtaaaaaacactgcacgtttcgtcggtcattaccccggttatttacgcgactcagattccatccttatcagtggtactatggatcgcctgactatcatcgaaggtatgccgggtcatattccggcctggcagcgcgaacaaggcttagtaaagcctggaggacgccgtcgtcgcctgagcggatcggagtcgaacatgcgccagaaggtcgatccgagcacagggccacgccgctcaacccgttcgggcaccgttaaccgctcgaatcagcgcactggtcgcaatggggacccgctgttagttgaaattcgtatgaaggaagactgggtcttattagacgcgcgcggtctgttacgtaacttgcgttggcgcgaaagtaagcgtggcctttcctgcgatcatgaggacctgtcgttgagtggcttattagcccttttctctggtgatccggtaattgatcccgtacgcaacgaggtcgtgttcttgtacggggagggcattatcccggtccgctccacaaaacccgttggaacccgccagtccaagaaactgctggagcgccaggcctcaatgggcccattaacattaatctcatgtgacttggggcagactaatcttattgctggccgcgctagcgcaatttcgctgacccacgggagcctgggcgttcgttcgtcggttcgcatcgagttggatcctgagatcatcaaatcctttgaacgcctgcgcaaggacgccgaccgcttagagaccgaaatcttaactgcagccaaagaaactttaagcgatgaacaacgtggagaggtgaacagtcatgaaaaagatagcccccaaaccgctaaggcgagcttgtgccgtgaacttggcttacatccgccaagtcttccgtggggacagatgggaccaagcaccaccttcatcgccgatatgttgatctcacatggccgcgacgacgatgcctttttgtcccacggggagttccccactcttgaaaaacgtaaaaaattcgacaaacgtttttgtctggaaagtcgtccattgttaagtagtgaaacccgtaaggcccttaatgaatcgttgtgggaggtaaagcgcacttcgtcggagtatgcccgtctgtctcaacgcaaaaaagaaatggctcgccgcgctgtaaacttcgtagtagagattagtcgtcgtaagactggtcttagtaacgttattgttaacattgaagatcttaacgtacgtatctttcatggaggcggaaagcaagctccgggttgggatggcttcttccgccccaagtcggaaaatcgttggttcatccaagctatccataaggctttctccgacttagctgcccaccatggtattccagtgatcgagagcgatccgcagcgcacatcgatgacctgtcctgaatgtggtcactgtgattctaaaaatcgcaacggtgtccgcttcctttgtaagggctgtggggcttctatggacgcagattttgacgcagcctgtcgtaacttagaacgtgtggcactgacagggaaacctatgcccaagccgtcaacctcatgtgagcgcttgttaagcgcaacgaccgggaaggtttgttcggaccactcattgtcgcatgacgcgattgaaaaggcatcataa

SEQ ID NO: 15 - DNA, 2382 bp, Artificial
  Bacteria optimized
  misc_feature (1)..(2382): cas_phi-5
atgagttctcttccgacacctctggaactgttgaaacagaagcatgcggacttgttcaaaggtttacagttttcatctaaagacaataaaatggccgggaaggtactgaagaaagacggcgaggaggccgcgttggccttcctgtcggaacgtggtgtaagtcgcggcgaactgcctaacttccgtccgccggcaaaaacgttggtagttgcccaatctcgtccatttgaggaattcccgatctatcgtgtatcagaggcaattcagctttacgtctactcattgagcgtaaaagaactggaaaccgtgcccagtggcagttcgaccaagaaagagcatcagcgctttttccaggactcttccgtacctgatttcgggtacacgtccgtccaaggacttaataagatctttgggctggcccgcggtatttacttgggggttatcacacgcggcgaaaatcaattacagaaggctaaatctaaacatgaagcactgaataagaagcgccgtgcctccggcgaagccgagactgagttcgacccaacgccttacgaatatatgacgcctgagcgtaaattggccaaaccgcccggagtcaatcacagtattatgtgttatgttgatatttctgttgatgaattcgatttccgtaatccggacggaatcgttttgccctcggaatacgccggttactgtcgcgagatcaataccgcgatcgaaaaggggaccgtcgatcgtcttggccatttaaaaggcggacccggttatattccaggccaccaacgtaaggagagcactaccgaagggcctaagatcaacttccgtaagggacgtattcgccgctcgtatacagcgttgtatgccaagcgtgacagtcgccgtgttcgtcagggtaagcttgccttgccttcttaccgtcaccacatgatgcgtttgaactctaacgcagaatccgcaattctggctgtaattttctttggaaaagactgggtcgtttttgatctgcgtgggttgttacgcaatgtacgctggcgtaatttatttgttgacggatcgactccgagtacgcttctgggcatgtttggagatcccgtgattgatcccaagcgtggggtagtcgcgttctgctataaggaacaaatcgtgcccgtcgtttcaaagagcatcacaaagatggtcaaggccccggagttgcttaataagttatacttgaagtctgaggaccctctggtacttgtagcgatcgacttgggtcagactaatcctgttggggtaggcgtgtaccgtgtcatgaatgcgtctttagattacgaagtcgtcactcgcttcgcccttgagtctgaacttttacgcgaaattgaatcttatcgccaacgtacaaatgctttcgaggcccagatccgcgcagaaacgtttgacgcaatgacaagtgaggagcaggaggagatcacgcgcgtccgcgctttcagcgcctctaaggctaaagagaacgtatgtcaccgcttcggaatgccggtagacgctgtggactgggcgacaatgggttctaacacgatccacatcgctaaatgggtgatgcgtcacggtgatcctagtcttgtggaagtactggaatatcgcaaagataatgagattaaattggataagaacggagtacctaaaaaggtgaaactgacggacaaacgtatcgctaatctgacgtctattcgccttcgtttttcgcaagaaacatcgaaacactacaacgataccatgtgggaacttcgccgtaagcaccccgtctatcagaagctgtcaaaatccaaagctgatttcagccgtcgcgtggttaactcgattatccgtcgtgtcaaccatctggtaccacgcgcacgtatcgtcttcattatcgaggaccttaagaatcttggaaaggtctttcacggcagtgggaagcgcgagttaggctgggactcatactttgaaccgaagtcggagaatcgctggttcatccaagttttacataaggcttttagcgagacaggtaagcacaaagggtattatattatcgagtgctggccgaactggactagctgcacatgtccgaagtgcagctgctgcgatagtgagaaccgccacggtgaggtattccgctgcttagcctgcgggtacacctgcaataccgactttgggactgcaccggataatcttgtcaagattgcaacgactggaaaaggattgccaggaccaaagaaacgttgtaaaggttcgtccaaaggaaaaaacccgaagatcgcacgttcatcagagaccggggtttcggtaaccgagtcgggtgcccccaaagttaaaaaatcatccccaactcaaacttcccaatcgtcttcgcagagcgctccataa

SEQ ID NO: 16 - DNA, 1326 bp, Artificial
  Bacteria optimized
  misc_feature (1)..(1326): cas_phi-6
atgaacaagatcgaaaaagaaaagacccccttggctaaattaatgaacgagaactttgccggtttgcgttttccattcgctattattaaacaggcaggcaagaaactgcttaaggagggggaactgaaaacgattgagtatatgaccgggaaaggtagcattgaacccttgccaaatttcaaaccgccagtaaagtgcttgatcgtggctaaacgtcgcgacctgaaatattttcccatttgcaaagcgtcatgtgagattcaatcgtatgtctattctcttaactataaggatttcatggactacttctcgacgcctatgacgtcacagaagcagcacgaggagtttttcaaaaaaagtggacttaatatcgaatatcaaaatgttgctggattgaatttgattttcaacaatgtaaaaaacacctataacggggttatccttaaagtaaaaaaccgcaacgaaaaacttaaaaagaaagccattaaaaataactatgagtttgaggaaattaaaaccttcaatgacgatggatgtcttattaacaagcctggtattaacaacgtgatttactgcttccaatccatcagccccaagatcttgaagaacatcacacatttacccaaggagtacaacgattatgactgctctgtcgatcgcaatattatccaaaagtatgtgagtcgtcttgacatccccgagtcgcaacccgggcacgttcccgaatggcagcgtaaattgcccgagttcaataacactaacaatccacgtcgtcgccgtaaatggtatagtaacgggcgtaacatctcgaaagggtattccgtcgatcaggttaatcaggccaaaatcgaagacagcctgttggcgcaaatcaaaatcggagaagactggattatcttggatattcgcggtctgcttcgcgacttgaatcgccgcgagcttatttcttacaagaataaacttaccattaaagatgtgttaggtttcttctccgactaccctattattgatattaagaaaaatttagtaacgttttgctacaaggaaggagtaattcaagtagtatcacaaaagtccattggaaacaaaaaatctaaacaattacttgaaaagttgatcgagaacaagccgattgctctggtttctattgatttgggtcagacaaacccagtctcggtcaagatctctaaattgaataagatcaacaacaaaatctcaattgagtcatttacgtaccgcttccttaatgaggagatcttaaaggaaatcgaaaagtatcgtaaagattacgataaattggaattgaagttgattaacgaagcgtaa

SEQ ID NO: 17 - DNA, 2319 bp, Artificial
  Bacteria optimized
  misc_feature (1)..(2319): cas_phi-7
atgtctaatacagcagtatcaacacgcgagcatatgtctaacaaaacaacgccaccgtcgccgttgagtttgttattgcgtgcgcattttccgggccttaaattcgaatctcaagactataagatcgctgggaaaaagcttcgcgatgggggcccagaagcggtaatcagctacttgaccggtaaaggacaagccaaattaaaagatgttaagccacccgcaaaagcgttcgtcattgctcaatcacgcccttttattgaatgggatttagtgcgtgtgagtcgccaaattcaggagaaaattttcggcatccccgcaactaaagggcgtcccaagcaagacggtctttcggaaacggcgttcaatgaggctgtggcgtcattagaggtagatggaaagtcgaagctgaacgaagaaacacgtgctgcattctatgaagtgttgggcctggatgcaccaagtctgcatgcacaggcacaaaacgccttgattaaatcggcaatttctattcgtgaaggcgtcttaaaaaaagtggaaaatcgtaatgaaaaaaatttatcgaaaaccaagcgtcgtaaggaggcaggtgaagaggccaccttcgtagaggagaaagcgcacgacgaacgcggctatcttatccaccctcccggtgttaaccagacgattcctggataccaggccgtggtgattaaatcttgcccgagcgattttatcggacttcctagcggctgcctggcgaaggagtcggccgaggccttaaccgattacttgcctcacgaccgcatgaccattcccaagggtcaacccggttacgttcccgaatggcagcatcccttacttaatcgtcgtaagaaccgccgccgccgtgattggtactcggcttctttaaataaaccaaaagcaacctgctctaagcgcagtggcacgccgaatcgtaaaaatagccgcacagaccaaatccagtcggggcgcttcaagggcgctatccctgtgctgatgcgttttcaggacgaatgggtaattattgacattcgtgggcttttacgtaacgcacgctatcgtaaattactgaaggagaaaagcacaatcccggatttgttatcgttgttcactggggacccctcaattgatatgcgtcagggggtatgtaccttcatctacaaagctgggcaggcgtgctctgctaagatggtaaaaacgaaaaatgctcctgagatcttgtcagagcttacaaagagcgggccggttgtgttagtttcgatcgatttgggacaaactaatccgattgcagcaaaggtgtcgcgtgttacacaattgagcgatggtcaacttagccatgaaacgttattacgtgaattgctgtcaaacgactcaagtgatggcaaagaaatcgcccgctaccgtgtggcttcagatcgtctgcgtgacaaattggccaacttggcggtcgagcgcttaagccccgagcataaatcagagattcttcgtgctaaaaatgacacaccggcgttgtgtaaagcccgcgtgtgtgcggcccttggtttaaatcccgagatgatcgcgtgggataaaatgaccccatatacggaattcctggcgacagcctatcttgagaagggcggcgaccgtaaggtggcgactttgaagcccaagaaccgcccagaaatgctgcgccgcgacatcaagttcaagggtaccgaaggcgtgcgcattgaggtgagtcccgaagcagcggaagcgtaccgcgaagcccagtgggacttacagcgtacctctccagagtacctgcgtcttagtacatggaaacaagagcttacaaaacgtatcctgaatcaactgcgccataaggcagccaagtcctcacaatgtgaagtagtggttatggcgttcgaagacttgaacattaaaatgatgcacggaaacgggaaatgggccgatggaggatgggacgcattttttatcaagaaacgtgaaaatcgttggttcatgcaggcattccataaatctttaaccgagcttggcgcacacaaaggagtccccactatcgaagtgacaccgcaccgcacttcgatcacctgtacaaagtgtgggcattgtgacaaggcaaatcgtgacggagagcgctttgcgtgtcaaaagtgcggcttcgttgctcatgcagatttagagattgctacagataatattgagcgtgtggctctgacagggaagcctatgccgaaaccggagtcagaacgttcgggagacgctaaaaaatccgtgggtgcccgcaaagcggcgtttaagccggaagaagacgctgaggcggcggaatga

SEQ ID NO: 18 - DNA, 2154 bp, Artificial
  Bacteria optimized
  misc_feature (1)..(2154): cas_phi-8
atgattaaaccgacagtgagccagtttctgacgccgggctttaagcttatccgcaaccattctcgcacggccggcctgaagctgaaaaatgagggagaggaggcctgtaagaagttcgtacgtgagaacgagattccaaaggatgaatgtcccaacttccagggtggcccagctatcgctaacatcatcgcaaaatctcgcgaattcacagaatgggaaatctaccagtccagtcttgcaattcaggaggtcattttcacattaccaaaagataagcttccggagccaatcttgaaggaggaatggcgcgcccaatggttgtcggaacacggcctggataccgtcccgtacaaagaagcagctggcttaaatcttatcatcaaaaacgcggtcaacacatataagggtgtacaggtaaaagtagacaataagaacaagaacaatttagccaagattaaccgcaagaatgaaatcgccaaattaaacggcgaacaggagatttccttcgaagaaattaaagccttcgatgataagggatatctgcttcaaaagcctagccccaataaatctatctattgttaccaaagtgtctcacctaaaccattcattacgagcaaatatcacaacgtaaatcttccggaagaatatattggatattatcgcaaaagtaatgaaccgatcgtctctccctatcaatttgatcgcttacgtatccctatcggcgagcctggatatgtgcccaagtggcagtatacatttcttagcaaaaaggaaaacaagcgtcgcaaattatcgaagcgcatcaaaaatgtctccccgattttaggcatcatttgtattaaaaaggattggtgtgtttttgacatgcgcggattattacgtaccaatcactggaaaaaataccacaaacccactgattccattaacgacttattcgactatttcacaggcgacccagtaattgacactaaggcgaacgttgtccgtttccgttataagatggaaaacggaattgtaaattacaaaccggttcgtgagaagaaaggcaaagagcttttagaaaatatttgcgatcaaaatgggtcgtgcaagttagccaccgtggacgtgggtcaaaacaacccggttgcaattggcttgtttgaattgaagaaagtcaatggtgagctgaccaaaactttaatctcacgtcaccccactccaatcgacttctgcaataagattaccgcctaccgcgaacgctatgacaagttggaatctagtatcaagttggacgcgattaagcagcttacttccgaacaaaaaatcgaagtcgacaattacaacaacaacttcaccccccagaacaccaagcaaattgtgtgttctaaacttaacatcaacccgaacgatcttccatgggacaaaatgattagcggcacacactttatctcagaaaaggcccaggtctcgaacaagtctgaaatctatttcacgagtactgataagggtaagacgaaggatgtgatgaagagtgattacaaatggttccaggactacaaacctaaattatctaaagaagtacgtgacgctctttccgacattgaatggcgtcttcgccgcgagtcgcttgagttcaataagttatccaagagccgtgagcaggatgcccgccaattggccaactggatttcgtccatgtgtgatgtgatcggcattgagaatttggtgaaaaaaaacaatttttttggaggctctgggaaacgcgaacccgggtgggataatttctacaaaccaaagaaggagaatcgctggtggatcaacgctatccataaagctttaacagaactttcccaaaataaaggtaagcgcgtgattctgcttcccgctatgcgtacttcaattacttgtcccaagtgtaaatactgcgattccaagaaccgtaacggagagaagtttaactgtttaaaatgcggtatcgaacttaacgcggatattgacgttgctacggagaacctggccacggtcgcgattaccgcacagagtatgcctaagccaacttgcgagcgcagtggcgacgcgaaaaaaccggtgcgtgcacgtaaggcaaaagctcccgagttccacgataaattggcgccatcttacaccgtggttttgcgtgaagcagtatga

SEQ ID NO: 19 - DNA, 2382 bp, Artificial
  Bacteria optimized
  misc_feature (1)..(2382): cas_phi-9
atgcgctccagccgcgagattggtgacaaaattcttatgcgccagcctgctgagaagactgctttccaggttttccgccaagaggttattgggacccaaaagctttcgggtggagatgcgaagactgcagggcgtttatacaaacagggcaaaatggaagctgcccgtgaatggttactgaagggtgctcgtgacgacgtgccgccaaattttcagccgcccgctaaatgcctggttgttgcggtttcccatccgttcgaagagtgggatattagtaaaacgaaccatgacgtacaggcttatatctacgcgcaacccttgcaggctgaaggacacctgaacgggttgagtgagaagtgggaggatacttcagctgaccagcacaagttatggtttgagaagaccggtgtcccagatcgcggcttgcccgtgcaagcgattaacaaaattgctaaggccgcagtaaatcgcgcgtttggtgtcgtacgtaaggtcgaaaaccgtaatgagaaacgtcgttcacgcgataatcgcatcgctgaacacaaccgcgaaaatgggctgactgaggtggttcgcgaggcacctgaagtggccaccaacgcagatgggtttctgttacacccgccagggatcgatcctagcatcttaagctatgcgtcagtatccccggttccctataattccagcaaacattccttcgtccgcttacctgaagaatatcaggcgtacaatgtcgaacccgacgcaccgattccgcagttcgttgtagaagaccgtttcgcaatcccgccaggacaacctgggtacgtccccgaatggcaacgccttaaatgttctactaacaagcatcgccgtatgcgtcaatggtctaaccaagactataagccgaaggcgggccgtcgtgcgaaaccgctggaatttcaggcccatttgactcgtgagcgcgcaaagggcgcactgctggtcgttatgcgcattaaagaagattgggtagtattcgatgtgcgcgggttacttcgtaacgtggagtggcgcaaggtgttgtccgaggaagctcgtgagaagctgactctgaaggggttgttggatctgttcacaggggaccccgttatcgataccaagcgtgggatcgttacattcctgtacaaggccgagatcacgaagattctgagtaagcgtacagttaagaccaaaaacgcccgtgatcttttgctgcgccttacagaaccaggcgaagatgggcttcgtcgcgaggtgggattagttgccgtcgacctgggacagacgcatcccatcgcggcagcgatctaccgtattgggcgcacttcggcaggggccctggagagcacggtcttacatcgtcaaggccttcgcgaggaccagaaggaaaaacttaaagaataccgtaaacgccataccgccttagacagccgtttgcgcaaggaagcgttcgaaacgttatcagtcgagcagcagaaagagatcgtcaccgtgtctggatcgggtgcacaaattacgaaagacaaagtgtgcaactacttaggggttgacccttccactctgccatgggaaaaaatgggctcttacacacactttatttcggatgactttctgcgtcgtggcggagatccaaatatcgtacacttcgatcgtcaaccgaaaaagggaaaagtctccaaaaaaagtcagcgcattaaacgttcagattcacagtgggtagggcgcatgcgccctcgcctgtcccaggaaactgcgaaagctcgcatggaggcggactgggcggcacaaaatgagaacgaggaatacaagcgcctggcccgttcgaagcaggaacttgcgcgttggtgtgttaatacgttactgcaaaatactcgctgcattactcagtgtgacgagattgtcgtggttatcgaggatttgaacgtcaaaagtttacatggtaagggagcacgcgaaccaggctgggataatttctttacaccgaaaacagagaaccgttggttcatccagattttgcataagacctttagtgaattaccaaagcaccgcggggagcacgtaattgaagggtgtcccttgcgcacttccatcacttgtccagcttgttcttattgtgataagaattcccgcaatggagagaaattcgtttgcgtagcgtgcggggcaacgttccacgcggacttcgaagtggcaacatacaatcttgtgcgtcttgccacgaccgggatgcccatgcccaaatcgttggaacgtcaaggtggtggagaaaaagctggaggtgcccgcaaggcacgtaagaaagctaagcaagttgaaaagatcgtggtacaggccaatgctaatgtcaccatgaacggagcctcgctgcacagtccgtaa

SEQ ID NO: 20 - DNA, 2439 bp, Artificial
  Bacteria optimized
  misc_feature (1)..(2439): cas_phi-10
atggatatgctggatacggagactaactatgcaaccgagactcctgcgcaacagcaagattattctcccaaaccgccaaagaaagctcagcgcgcaccaaaggggtttagcaaaaaagcgcgcccagagaaaaaaccgcctaaacccattacattgttcacccaaaagcatttcagcggggtccgcttccttaaacgcgttatccgtgatgcttcaaaaattttaaagcttagtgaatctcgcaccattacttttttagagcaggcgatcgagcgcgatggtagtgcaccacccgatgtgacaccaccagtacataacacgattatggctgtgacacgtcctttcgaggaatggccagaggtgattttgtccaaagctctgcagaagcactgctatgcactgaccaagaagattaaaatcaaaacatggcctaagaaaggtcccggaaaaaaatgcctggccgcttggtccgctcgtacgaaaattccgcttatccccggccaagttcaagctacaaatgggttgtttgaccgcatcgggtcaatttatgacggagtcgagaaaaaagttacaaatcgcaacgcaaacaagaagttagagtatgacgaggcaattaaagagggtcgtaacccggccgtacccgagtatgagacggcctataacatcgatgggaccctgattaataaaccagggtacaacccgaatttatatatcacacagtctcgtactccccgcttaattactgaagccgatcgccctcttgtagaaaagatcttatggcaaatggtggaaaagaaaacgcaaagccgtaatcaagcacgccgtgcacgcttagagaaggcggctcatttgcagggattgccggtgcccaagttcgttcccgaaaaggttgatcgctcccaaaaaattgagatccgcatcattgatcctttggataaaattgagccctacatgccgcaagaccgcatggccatcaaagcaagtcaagacggccatgtgccctattggcaacgtcccttcctgtcgaagcgtcgcaaccgtcgtgtgcgtgcggggtggggaaaacaagtcagctccatccaagcgtggttaaccggtgccttattagtaattgtacgcctgggaaacgaagcttttttagccgacattcgcggggcgcttcgcaatgcccagtggcgtaaactgcttaagccagacgcaacctatcagtcattatttaacctgttcacaggtgaccccgtcgttaacacacgtacgaaccaccttacaatggcataccgtgaaggcgtagtcaatattgtcaaaagccgctccttcaagggacgtcaaactcgcgaacacttacttacactgctgggccagggcaaaactgttgctggtgtttcgttcgatctgggacaaaaacatgcggcggggttgcttgccgcacattttggcctgggcgaagatggtaatccagtcttcactcccatccaagcttgctttttaccgcagcgttatcttgacagccttactaactatcgcaaccgctatgatgcccttacgctggacatgcgtcgtcagtctttgttggccctgacccctgcacaacaacaagagttcgcggacgcgcagcgtgatccgggcggacaggcaaaacgtgcgtgttgtttaaagcttaatttaaacccggacgaaattcgctgggacctggtatcagggatctctacaatgatctctgatttatacattgaacgcggtggagaccctcgtgacgtacaccaacaggtggaaactaaaccgaaaggcaagcgtaaaagcgaaatccgcatcctgaagatccgtgatggtaaatgggcctatgactttcgtcccaagattgctgatgaaactcgcaaagcacaacgtgaacagttatggaagcttcaaaaggcatctagtgagttcgaacgtctttcccgctacaaaattaatatcgctcgcgccattgcaaattgggctctgcagtggggccgcgagttaagtggctgcgacattgtgatcccggtacttgaagacttgaatgtcggctctaagttttttgacggaaaggggaagtggcttttaggatgggataatcgctttactccaaaaaaggagaatcgctggtttattaaagtgcttcacaaggcggttgcggagctggcaccccaccgcggagtaccggtctatgaagtcatgcctcatcgcacatccatgacgtgtcctgcttgccattactgtcatccgacaaatcgtgagggcgatcgtttcgaatgccagagttgtcatgtagttaagaacactgaccgcgacgtggcgccctataatatccttcgcgtcgcagtagagggaaagactcttgatcgctggcaagctgagaaaaagccgcaagcggagccagatcgcccaatgattttaattgacaaccaggaatcctga

SEQ ID NO: 21 - DNA, 2124 bp, Artificial
  Human optimized
  misc_feature (1)..(2124): cas_phi-1
atggcagatacaccaacattgtttacacaatttctccgccaccatctgccggggcaacgatttagaaaggacatccttaaacaggcgggacgcatccttgctaataaaggtgaggatgcaacaatcgcgtttttgcggggaaagagtgaggaatctccacccgattttcagccccccgttaaatgcccgataatcgcatgctcccgcccgctcacggagtggccaatttaccaagcatcagttgctatccaaggttacgtctatgggcagtccttggccgaatttgaagccagtgaccccggatgctcaaaagatggtctgctggggtggttcgacaaaacaggcgtttgcaccgattacttctctgttcaggggttgaatctcatttttcagaacgctcggaagcggtatatcggtgtgcaaacaaaggttacgaatagaaatgaaaagcggcacaaaaagctgaaacgcatcaacgctaagcgaattgcagaaggtctgcccgaattgacgagcgacgaacccgaatccgcgttggatgagaccgggcacctcattgacccccctggtcttaacacgaacatctactgttaccaacaggtaagcccgaagcctctcgcacttagcgaggtcaatcaactcccgacggcctacgctggatactcaactagcggtgatgacccgatacagccgatggtgacaaaggacaggttgagcatctcaaagggccagcctggctatattccagagcatcaaagagcattgctgtcccaaaagaaacatcgcagaatgaggggatatggactgaaggctcgcgccctgttggtgattgttcgcattcaggatgactgggccgtcatagaccttagaagcttgctcaggaacgcctactggcgccgaattgtccagaccaaagagcctagcaccattacaaaactgttgaaactggtgacaggtgatccagtgttggacgctacgaggatggtagctactttcacctataagccggggattgtgcaggtgcgctccgccaaatgtttgaaaaataagcaaggctccaaactgttctcagagcgctatcttaatgagactgtaagtgttacatctattgatttgggatcaaataaccttgtcgctgtagccacttatcgcttggttaacggaaacactcccgagcttctgcagaggtttactctcccgagtcacctcgtgaaagactttgaacgatacaaacaggcgcatgatacattggaggactccatacagaagactgcagtggcgagcttgccacagggccaacaaacggaaatccgcatgtggagtatgtacggcttccgcgaagcccaggaacgcgtctgtcaggaattgggcttggcagatggatcaatcccttggaacgttatgactgcgaccagcaccatactcacagatcttttccttgctcgcggtggtgatcccaaaaaatgtatgtttactagtgaacccaagaagaagaagaatagcaaacaggttttgtataagatacgagaccgagcttgggcgaagatgtataggacattgttgagtaaggaaacccgggaggcatggaacaaggcactctggggtcttaagaggggctcccccgattatgctaggctctcaaagaggaaggaggaacttgcgagaagatgcgtaaactacactatcagcactgctgagaagcgggcgcagtgcgggaggaccattgtagcgctggaggatcttaatatcggatttttccatgggcgggggaaacaggaaccgggttgggttggccttttcaccagaaagaaggaaaatcgctggctcatgcaggccttgcataaggcattcttggagctggctcaccacagagggtaccatgtcattgaagtcaatcccgcctatacttctcagacttgtcctgtgtgcaggcactgtgatcccgacaaccgagaccagcataatcgcgaggcctttcattgtatcggatgtggtttccgagggaatgcagaccttgacgtggcgacccacaatatagccatggttgccatcacgggagaatctttgaaacgcgctcgcggtagcgtggcgtcaaagaccccacaacccttggccgcggaatag

SEQ ID NO: 22 - DNA, 2274 bp, Artificial
  Human optimized
  misc_feature (1)..(2274): cas_phi-2
atgccaaagccagcagtagagagtgagtttagtaaagtgctgaaaaagcacttccccggggaacggttcagatcaagctacatgaagagaggaggcaagatattggcagcccaaggggaggaagctgtcgtagcttacctccagggtaaaagcgaagaagagcccccaaacttccaacctccggcgaaatgccacgttgtcacaaagtccagagattttgcggaatggcccatcatgaaggcaagcgaagctatacagcggtacatttacgccctgagtaccacagaacgagcggcatgtaagcccgggaaaagttctgagtcacacgcagcttggttcgcagcaactggcgtgtctaatcacggctattcccacgtgcaagggctcaacttgatctttgaccacactctcggccggtacgacggggtacttaaaaaagttcagctccgcaatgaaaaggcgagggcgaggctcgagagtataaatgctagcagagctgatgagggactgcctgaaataaaggcggaagaggaggaagtggctaccaacgaaacaggtcatcttttgcagcctccgggtattaacccttccttttatgtctaccagacgatctccccacaagcttatcgcccaagagatgagattgtattgccaccagagtatgcgggttacgtacgggaccctaatgctcctatacctctcggtgtcgtgcgaaaccgctgcgacatacaaaaaggctgcccaggctacattccagagtggcaacgggaggcaggaacggcaatttctcctaagacgggtaaagctgtgactgtacccggtctgagcccgaaaaaaaataagcggatgcggcgatattggcgatctgagaaggaaaaggcacaggacgccctcttggtaacagttaggataggtacggattgggtcgttattgatgtacgagggcttctcagaaatgcgagatggagaacgatagccccaaaagatataagcctcaacgcgctcttggacctctttaccggtgacccagtaatagatgttcggcgaaacatcgtgacgtttacttacactctcgatgcatgcggtacgtatgcaagaaagtggactcttaaaggcaagcaaacgaaagctaccctggacaaactgactgccactcagactgtcgcccttgtggcgattgatcttggccaaacgaacccaatctctgcgggcataagccgcgtgacgcaagaaaacggagcactgcagtgcgaaccgctcgacagattcactttgccggatgaccttcttaaggatatatctgcttacagaatagcttgggaccggaacgaggaagaattgcgagcgcggagtgtagaagcgctgcccgaggcccagcaagctgaggtcagggcactcgatggtgtgagcaaggagaccgctcgaacgcaactttgtgcggactttgggctcgatcctaaacgactcccctgggataagatgtcctccaacaccacttttatctcagaagcactcctctcaaatagtgtctctcgggaccaggttttttttacccctgctccaaaaaaaggcgctaagaagaaagccccagtagaggtcatgcggaaagaccgaacgtgggctagagcctacaaaccccggcttagtgtagaagcgcaaaagctgaaaaacgaagctctctgggccttgaagcggacgtcccctgaatacctgaaactcagtcgacggaaagaggagctctgtagacgatccatcaattatgtgatcgaaaagactcggaggagaacacaatgtcaaatagtaatccccgttattgaggatcttaacgtcaggtttttccacggtagcggcaagagattgcctggctgggacaattttttcacggcaaagaaggaaaatcggtggttcatacagggtctgcacaaagcgttttcagatctccggacacatagatccttttacgtctttgaagtgcgaccggaacgcacaagtattacttgtccgaaatgcggccactgcgaggtcggtaaccgcgacggagaggcattccaatgcttgagctgtggcaaaacgtgtaacgcggacctcgacgtagccacccataaccttacgcaagtggcgttgactgggaagacgatgcctaaaagggaggaacctcgcgacgcgcaagggactgctccagcacgaaaaacaaagaaagcttcaaaatcaaaggcgcctccggctgagcgggaagatcagacgcccgcgcaggaaccttctcaaacttcttag

SEQ ID NO: 23 - DNA, 2301 bp, Artificial
  Human optimized
  misc_feature (1)..(2301): cas_phi-3
atggaaaaggagattacggaactgacgaaaattcgccgcgagttcccaaacaagaaattcagcagcacagacatgaagaaggctggcaaattgcttaaagctgaaggcccagatgccgttcgcgattttcttaacagctgccaggaaatcattggcgacttcaaaccgcccgttaagactaatattgtaagcatctctcgaccttttgaggaatggccggtatctatggttgggcgagcgattcaggaatactactttagtctcactaaagaggagctcgaaagcgtgcatccaggtacgtccagcgaggatcacaaaagcttctttaatattacagggctcagtaactataattatacatcagttcaaggcctgaatctcatttttaagaacgccaaagccatatatgatggcactcttgtcaaagctaacaacaaaaacaagaaattggagaaaaaattcaacgaaattaaccataaaaggtcactggaaggcctccctattattacacctgatttcgaagagccgttcgatgaaaacggacaccttaacaaccctcccgggataaacagaaatatttacgggtatcaaggttgtgcggctaaagtcttcgtccctagtaagcacaagatggtatccctccccaaagaatatgaggggtacaatcgagatcctaatctcagtctggcaggcttccgcaatagacttgaaattccggaaggggaacctggacatgtcccttggttccaacgcatggatatcccggagggtcaaatagggcacgtaaacaaaatccagagattcaacttcgtacacggtaaaaactccggaaaggtgaaattctccgataagacgggacgggtcaagcgctaccaccattccaaatataaagatgccaccaagccgtataaatttctcgaagaatccaagaaggtctcagcgctcgatagtatacttgcaataataacaattggcgatgattgggtagtctttgatattcgagggctctaccgaaatgttttttatagagaattggcgcagaaaggtcttacggcagtgcagctcttggatctctttacgggggacccggtaatagacccgaaaaagggtgtcgtaaccttcagctacaaagaaggtgtcgtcccggtattctctcagaaaatagtcccccgctttaagtcaagagatactctggaaaagcttacttctcaagggccggttgcgctcctcagtgtagacttgggacaaaacgaacctgttgcggcgcgggtgtgctctttgaagaacattaacgacaaaataacacttgacaattcttgtcggatttcattcctggatgactataagaagcagataaaagactaccgagactccttggatgagctggaaataaaaattagattggaggctattaattctcttgagacaaaccagcaggtggagatcagagatctggatgttttctcagctgatagggccaaagctaacactgttgacatgtttgatattgatccgaatctcatatcctgggattcaatgtctgatgcccgagtatcaacccagattagtgatttgtacctcaaaaatgggggggatgaatctagggtgtatttcgagattaacaataagcgaataaagagatctgattacaacataagtcagttggtcagaccaaagttgtccgattccactagaaagaatttgaacgattctatatggaagcttaagaggacgagcgaagagtatctgaagctgagtaaacgcaagcttgagcttagcagggccgtagtcaattatactatccggcagagcaaattgctttcaggaatcaacgacattgttataattctcgaggacctcgacgtgaaaaagaaatttaatgggaggggcatccgcgatattggatgggataacttcttcagtagcaggaaagaaaataggtggtttatacctgccttccataaggcctttagtgaactcagcagcaacaggggcctctgcgtgatcgaggtaaacccggcgtggacatcagcaacttgtcctgactgcgggttttgttctaaggagaaccgggatggaatcaactttacttgccgaaagtgtggcgtgtcctatcacgctgacatcgatgtcgctacacttaatatagcccgagttgccgtacttggtaaaccaatgagtgggccagctgatcgcgaaagactgggcgacacgaaaaaaccaagggtggctcgatccaggaaaaccatgaaacgcaaggatatttcaaactccactgtcgaggcaatggtaaccgcatag

SEQ ID NO: 24 - DNA, 2298 bp, Artificial
  Human optimized
  misc_feature (1)..(2298): cas_phi-4
atgtattcccttgaaatggcagacttgaaaagtgaaccgagcttgttggcgaagctgttgcgcgatcggtttcctggcaagtactggttgcccaaatactggaaacttgcggagaagaaaagattgactggaggagaggaggcagcttgtgaatatatggcggataagcagctcgactcaccgcccccaaacttcaggccccctgcacgatgtgtaatattggcaaagtccagaccattcgaggattggcccgtgcaccgcgttgcgtctaaagcacaatcttttgtaatcggactgagcgagcaaggttttgcggcacttagggccgcaccacccagtacagcagatgctaggagagactggttgcggagccacggcgcctccgaagatgaccttatggctcttgaggcgcagctcctcgagacaatcatgggtaatgcaattagccttcatgggggcgtactcaaaaagatagacaatgctaatgttaaggcagcaaagcggctctccggccgaaacgaagcgaggctcaacaagggattgcaagagcttccccctgaacaggaaggaagtgcgtatggggcggatggtcttttggtgaatcccccgggtcttaatttgaacatctactgcaggaaaagctgctgtccaaaacccgtgaaaaacactgccaggttcgtggggcactaccctggatacctgcgagacagtgacagcattttgattagtgggaccatggaccgactcaccataatcgaagggatgccagggcatattccagcatggcagcgagaacaaggtttggtgaaaccaggcgggcggcggcgcaggcttagtgggtcagagtccaatatgaggcaaaaagtggacccgagcacgggaccccggcgaagtacgcggagtgggactgttaaccgatctaaccagcggacagggagaaacggtgacccgttgcttgtcgaaatacggatgaaagaagactgggtccttttggacgcgagggggctgttgcgcaacctccgatggcgagaaagcaagagaggtctcagttgtgaccacgaagatctgtccttgagtggtctcctcgcgctgtttagtggtgatccagtaattgaccccgtccggaacgaggtagtattcttgtacggtgagggaatcattcccgttcggtcaaccaaaccagtagggacgcgccaaagcaaaaagctcctcgagaggcaggcatccatgggccctttgacactgatctcttgcgatttgggacaaaccaacctgattgcagggcgggcgtctgctatctcacttacacacggaagcctgggtgtaagatcctccgtcaggattgagcttgacccagaaatcataaaatccttcgagagattgcgcaaggacgcggataggcttgaaacggaaatcctgactgcggcaaaagaaactttgagcgacgagcaacgaggtgaggtaaacagtcacgaaaaggactccccccaaactgcaaaagcctccctttgccgagaactcggcctccaccctccatctttgccgtgggggcagatgggtccctccacgacttttatcgccgacatgcttatcagtcacggtcgggatgacgatgcattcttgtcccatggcgagtttcctactctggaaaaaagaaagaaatttgataagagattttgtcttgagagtcggccgttgctctctagtgagacccgcaaagctctgaatgaaagtctgtgggaagttaagcggaccagttctgagtacgcaaggctcagccaaaggaaaaaggagatggcgagacgagcagtaaattttgtcgtagaaataagccggcgaaaaacaggactttccaacgtgatcgtgaacatcgaagatcttaatgtgcggatttttcatggaggcggtaaacaggcgccaggatgggatgggttttttcggccaaaaagtgaaaaccgctggtttatccaagctatccacaaagcattttccgacttggcagctcaccatggaattcctgtaattgaatccgatccacaaagaacatccatgacttgcccggaatgtggtcactgtgactcaaaaaatcgcaatggcgtacggttcctgtgcaaaggatgtggtgcttccatggacgcagactttgatgccgcatgtaggaatttggaacgggtcgcgctcacgggcaaaccgatgcctaaacctagcacctcctgtgagcggcttctctccgccaccacgggtaaagtatgtagcgatcattccctttcccatgatgcaatagaaaaagcgtcttag

SEQ ID NO: 25 - DNA, 2382 bp, Artificial
  Human optimized
  misc_feature (1)..(2382): cas_phi-5
atgtcatcccttcccactccgctggagttgttgaaacagaagcacgcggacctcttcaaaggtcttcaattctcaagcaaggacaataagatggccgggaaggtcttgaaaaaggatggcgaagaggcggctctcgcattcttgagtgaaaggggggttagccgcggtgagctgcccaacttccggccaccggcgaaaacccttgtggtcgcgcaatcaagaccctttgaggagtttcccatatatcgggtgagtgaggctattcaactctatgtatactcacttagcgtcaaggaacttgaaacagttccatcagggagttccacgaaaaaagaacaccagaggttttttcaagattcaagcgttcccgattttggctatacctccgtgcagggtttgaataagattttcggtctcgctagaggcatatacttgggtgtcatcacaaggggcgaaaaccaattgcagaaggcgaaatcaaaacatgaggcgctcaataagaaacgccgcgccagcggcgaggcagagacggagtttgatccaacaccatatgaatatatgactcctgagaggaagctggcgaagccccccggtgtaaatcatagtattatgtgctacgttgatatctctgtggacgaatttgactttaggaacccggatggaattgtgctgcctagtgaatacgccggatactgtagggagatcaatactgcgatagaaaaaggtactgtagacaggctcggccatttgaaaggtggccccggatacatacctggacaccagaggaaagaatctaccacggaaggtcctaaaattaacttccgcaagggtaggataagacggtcatatacagcactgtatgccaagcgagattcccggcgcgtccgccaggggaagctggctcttccttcctataggcatcatatgatgaggctcaactccaatgcagaatccgccatcctggcggtcatattttttggtaaagattgggtagtgtttgatcttcgcggactcttgaggaacgtgcgatggcgcaaccttttcgtggatggcagcactcccagtacactcttggggatgttcggagatcccgtgatcgacccaaagcgaggcgtcgtagcgttttgctacaaggagcagatagttcctgtggtgagtaaaagtataacaaaaatggttaaggcacccgagttgctgaataagctgtatctgaaaagtgaggatccccttgtccttgtcgctattgatcttggtcagaccaaccctgttggcgtcggcgtttacagagtaatgaacgcttctctggattatgaggtcgtgacgagatttgcccttgaatctgagcttcttagggagattgaaagctatcgccaacgaactaacgcgttcgaggcccagattcgagccgaaacctttgatgcgatgacatcagaggagcaagaagagattacacgggttagagctttctcagcgagcaaggcaaaagaaaatgtttgccatagatttggcatgccggtagatgctgttgattgggccacaatgggtagtaacaccattcatatcgccaagtgggtaatgagacatggcgacccaagcctcgttgaagtactcgagtatcgcaaagataatgaaataaaacttgacaaaaatggggttccgaaaaaagtgaagctcaccgataaacgcattgctaaccttacgtcaattagattgcgatttagtcaagagacaagtaaacactacaatgacaccatgtgggagttgcgacggaaacatcctgtttaccagaagctctccaagagtaaggcagatttcagtagaagagtggtgaattccataatacgccgagtcaaccatcttgttccccgcgcccggatagtttttataattgaggatttgaaaaaccttggcaaagtatttcacggctcagggaaaagagagctcggttgggatagttatttcgaaccaaagtctgagaacaggtggttcattcaagtacttcataaagcgtttagtgagacgggtaagcataagggatactatatcatagaatgctggcccaactggacttcctgtacatgtccaaaatgtagttgttgtgacagcgaaaaccggcacggggaagttttcagatgcctggcgtgcggctacacttgcaatactgattttggcactgcccctgataaccttgtcaaaattgcaacgacggggaaaggcctcccgggaccaaagaagcgatgtaaaggatcttctaagggtaagaacccgaagattgcaaggtcatcagaaactggggttagcgtcacggagtctggggcaccaaaagtcaaaaaatcatctccaactcaaacttcccagtcatcctcccagtcagcgccctag

SEQ ID NO: 26 - DNA, 1325 bp, Artificial
  Human optimized
  misc_feature (1)..(1325): cas_phi-6
atgaataaaatagagaaagaaaaaacgccacttgctaagctcatgaacgagaattttgcaggcttgaggtttccgtttgccatcataaaacaggcaggcaaaaaattgctcaaagaaggcgaactgaaaaccatagaatacatgacgggaaaggggagtatcgagcctctgccaaattttaaaccacccgtcaaatgcctgattgttgctaaacgccgagatttgaaatacttcccgatctgcaaagcctcttgcgagatacagtcctacgtctattcacttaactacaaggactttatggattatttttcaacacctatgacatcccagaagcagcatgaagaatttttcaaaaagagcgggcttaacatcgaataccagaacgtcgctggtcttaatctcattttcaacaatgtaaaaaatacatataatggagtcatacttaaagtcaagaacagaaatgagaaattgaagaaaaaggctataaagaataactacgaattcgaggaaattaaaacgttcaacgacgatggatgtctcatcaacaagccgggaataaataatgtgatttattgctttcaatccataagcccgaagatccttaagaatattacgcacctcccgaaggaatataacgactacgactgctctgtggatcgcaacattatccaaaaatacgtaagccgattggacatcccggaatctcaacctgggcacgtgcctgaatggcagcgcaaattgccagaattcaacaacacaaataatcccagaagacgccggaagtggtactctaatggtagaaatatatccaaaggttactctgtcgatcaagtaaaccaggcgaagattgaagacagcttgctcgcccagataaagatcggggaagactggatcattttggacatacgaggactgctccgagatctcaacaggagagagctcattagctataagaacaaactgacgataaaggacgttcttggatttttttcagattaccctataatcgacatcaaaaaaaaccttgtaacgttctgttacaaagagggggttattcaagtagtctctcagaaaagtattggaaataaaaagtccaaacaattgttggagaaactcattgaaaataagccgatagctcttgtttccatcgacctcggacaaacaaatcctgtaagtgtgaagatctctaagttgaacaagataaacaacaaaataagcatagagtccttcacttacaggttccttaacgaggaaatcctgaaagagatagagaaatataggaaggactacgacaagctcgaactgaagctcattacgaggcttag

SEQ ID NO: 27 - DNA, 2319 bp, Artificial
  Human optimized
  misc_feature (1)..(2319): cas_phi-7
atgtccaacacagccgtcagtactagggaacacatgtcaaacaagacgacacccccttctccgctttcattgctgttgagagcgcattttcccggactgaaatttgagtctcaggattacaagattgccggcaaaaaacttcgcgatggtggtcctgaagcagtgatttcatatctgacaggaaagggtcaagctaaacttaaagacgttaagcctccagccaaggcttttgtcatagctcaatctagaccatttatagaatgggacctggtacgcgtaagccgacagattcaggaaaaaatatttggcatcccagcaactaaggggcgaccaaagcaagatggtttgagcgagaccgcattcaatgaagctgtagcatctctcgaggttgatggaaaatccaagcttaacgaggagacgagagcagcgttttatgaagttcttggactggacgctccctccttgcacgctcaagcccaaaatgctcttataaaatccgctatttcaatacgggagggcgtgctcaaaaaagtagaaaacagaaatgagaagaacctctccaaaacgaaacggcggaaagaggccggtgaagaagcaacattcgtggaggagaaagctcacgacgaaagagggtacctgattcatcctcccggggtcaaccaaacgattccagggtatcaggcggtggttataaaatcctgccctagtgactttattggcctgccctcaggttgccttgctaaggagtcagcagaagcactgacagactatttgccgcatgaccggatgaccatcccaaaggggcagccgggttacgtgcctgagtggcagcatcctttgttgaataggcggaaaaacaggagaagaagggactggtattcagcgtcactcaacaaaccgaaagcaacgtgcagtaagcgctccggtactccgaataggaagaactctagaacagaccagatccaatctggccgctttaaaggagctattcctgtacttatgcgcttccaagatgaatgggtcatcatcgatattcgggggttgcttaggaatgcgagatatcggaaactgcttaaagagaaatccacgatccccgatcttctttcactttttacaggtgatccaagcattgatatgaggcagggtgtttgtacctttatatacaaagcaggtcaagcctgctcagccaagatggttaagactaaaaatgcgccagagatcctgagcgagctcacaaagtctgggccggttgtactggtttccattgatcttggccaaaccaaccccattgcggcaaaggtgtcacgggtaacgcagctttctgatggccaactctctcacgaaacgcttttgcgggaacttctcagtaatgactcaagtgacgggaaagaaattgcaagataccgagtggcgtcagataggctgcgggataagttggcaaacctcgcagtcgagaggctttccccggaacataaaagcgagatacttcgcgcaaaaaatgacaccccggcactgtgtaaagctcgagtttgcgcagctctgggattgaacccagaaatgatagcatgggacaaaatgactccctataccgaatttctcgcgaccgcttatcttgagaaggggggagacagaaaggtagcaaccctgaaaccgaagaaccgcccggaaatgttgcgaagagacataaaatttaaaggaacagaaggtgtgagaatcgaggtctcaccagaggctgcggaagcttacagagaagcccaatgggatctgcagcgcacgagtcccgaatatttgcggctgagtacatggaagcaagaacttacgaaacgaatcctcaaccaactgcgccataaagccgcaaagtcttcacaatgcgaggtggtggtgatggcgttcgaagacctgaatattaagatgatgcacgggaacggtaaatgggcagacggtggatgggacgctttttttatcaaaaagcgagaaaatcgctggttcatgcaggcatttcataaatccttgactgagctgggtgctcataaaggagtacccacaatagaggtaactccgcatcgaacgtccattacatgcactaaatgtggtcactgcgataaagccaacagggacggcgaacgattcgcatgccagaaatgtggattcgtagcccatgcggaccttgaaatcgctaccgacaatatcgagagggtcgcgctgactggcaaacccatgcctaagccagaatccgaacgaagtggggacgcaaagaaatcagttggggcacggaaggccgccttcaagccggaagaggatgcagaagccgctgaatag

SEQ ID NO: 28 - DNA, 2154 bp, Artificial
  Human optimized
  misc_feature (1)..(2154): cas_phi-8
atgataaaaccaacggtcagtcaattcctcacgccgggtttcaaactcatacgcaaccactcccgcacagcgggccttaagttgaagaacgaaggggaagaggcctgcaaaaagttcgtaagggagaacgagatccctaaagacgaatgtcctaacttccaaggcggccctgcaattgccaacatcatcgccaagagcagagaatttactgaatgggagatttaccaaagcagcttggcaatccaagaggtaatttttacacttcccaaggataagttgcccgaaccgatcctcaaagaggagtggagggcgcaatggcttagcgaacacggcctggatacggtgccttacaaagaagcagcgggcttgaatttgattataaagaatgccgttaatacctataagggtgttcaggtaaaagtagacaacaaaaataaaaacaatctggcaaaaattaataggaagaacgagatcgctaagctcaatggagaacaagagatctccttcgaggaaatcaaagcctttgatgataaaggctatctccttcagaagcctagcccgaataagagcatttactgttatcagtccgtatctcctaaaccatttattaccagtaaatatcataatgttaacttgcctgaagaatatattggatactacaggaagtcaaacgagcctatcgtgagtccatatcagtttgacagattgagaatacccattggcgagcccggttatgtgcccaagtggcaatatacattcctttccaagaaggagaataaacggcgaaaactgtcaaaacgaataaagaacgtctcacccatactcggcatcatatgcattaagaaggactggtgtgtcttcgatatgcgcggacttctccggaccaaccactggaagaaataccacaaacctaccgattccatcaatgacctgttcgattacttcacaggagatcccgtcatagacacgaaagcgaatgtggttcgcttcagatacaaaatggagaacggaatcgtaaactacaagcccgtgcgcgagaaaaagggcaaagaactcctggaaaacatatgcgaccagaatgggtcttgtaaacttgccactgtggatgtcggccagaataatcctgttgctataggcctttttgaattgaaaaaggttaatggcgaactcacgaagacgctgatttctcggcatcccactcctatagacttttgtaataaaatcaccgcttaccgggagaggtacgataaattggagtccagcattaaactcgatgccattaaacagcttacatcagaacagaaaatagaagtagacaactacaataataactttacaccccagaatacgaaacaaatcgtatgctcaaagcttaacattaacccgaatgacctgccatgggataaaatgatctccggcacccacttcatatccgaaaaggcgcaagtttcaaacaagtctgaaatatatttcacgtctacggacaagggcaagaccaaggacgtcatgaagtcagactataagtggttccaagattacaagccaaagctcagcaaagaagtgcgagatgccctgagcgatatcgagtggaggctcaggagggaatcactcgagtttaacaaattgtccaaaagtcgggaacaggacgcacgacaactcgctaactggatttcttctatgtgtgacgtaattggtattgaaaacctcgtaaagaagaacaatttttttgggggtagcggaaaacgggaacctggctgggataatttctacaagccgaaaaaggaaaaccggtggtggattaatgcaatccataaagcattgactgaactcagccaaaacaaaggtaagcgggttatcttgctcccggcgatgcgcacctctataacatgccccaaatgtaaatattgcgactccaaaaatcgcaacggcgaaaagttcaactgtctcaagtgtggcattgagctgaacgcagatatagacgtggcgacagagaacctcgcaacggtcgcgataaccgcgcaaagtatgccgaagcccacatgtgaacgcagtggagacgctaaaaaaccagtccgggcaaggaaggcgaaggcgcctgaatttcacgacaagttggcgcccagttatacggtggtcttgcgggaagctgtgtga

SEQ ID NO: 29 - DNA, 2382 bp, Artificial
  Human optimized
  misc_feature (1)..(2382): cas_phi-9
atgcgatccagtagagaaataggcgataagatcctgatgcggcagcccgccgaaaagaccgcctttcaagtttttcgccaggaagtcattggcacccaaaagttgtcagggggcgacgcgaaaacagccggtagactgtacaaacaaggcaaaatggaggcggcaagagagtggctcctcaagggcgcgcgggatgacgtccctccgaatttccaaccacctgctaaatgtcttgttgtcgccgtgagtcacccttttgaagaatgggatattagcaaaactaatcatgacgttcaagcctatatctacgcccaacccttgcaggctgaaggacaccttaatggcctgagcgaaaagtgggaggacacaagcgccgaccagcacaaactttggttcgagaaaactggcgtgccggacaggggcttgccagtgcaggctatcaacaagatcgcaaaagcagcggttaaccgcgcgtttggagtagttaggaaagtcgagaacaggaacgagaagaggcggtcacgagataatcggatagcagagcacaatcgggagaatgggctgaccgaggtcgtgcgagaagctccagaggtagcaacgaacgccgatggcttcctgcttcatccgccaggaatcgacccttccatactttcatacgcatcagtatcccccgttccctacaattcctccaaacatagttttgtcagactccctgaggagtatcaagcttacaatgtcgagccagatgctccaattccacaattcgtcgtagaggatagatttgctattccacccgggcagcccggttacgttcctgagtggcaacgattgaaatgttccaccaataaacatcgccgaatgcgacaatggtccaatcaagactacaagcccaaagccggccgacgagcaaaaccgctggagtttcaagctcatcttacgagggaacgagcaaaaggtgccctgctcgtcgtaatgcgcattaaagaagattgggtagttttcgacgtgagaggcctgttgcgcaatgtggagtggaggaaggttctctctgaagaggcaagagaaaaattgacgttgaaggggctccttgacttgtttacaggagatccggtaattgatacaaaacggggaatcgtaacttttctttacaaggcggagatcactaaaatattgtccaaacgaacggtgaagactaagaacgcgcgcgatttgttgctccgcctcacagaacctggagaagacggattgcgacgggaagtcgggcttgtagccgtggatcttgggcagacacacccgatagctgcagcgatctatcgcattgggcgaaccagcgcaggagctctcgaatccacagttcttcaccgccagggtttgcgggaggatcaaaaagagaagttgaaagagtaccggaaaagacatacagcgttggatagtcggcttagaaaggaggccttcgaaacgttgtcagttgagcagcagaaagaaattgtaaccgtaagtgggagtggtgcccaaatcacgaaagacaaggtctgcaactatcttggcgtagatccctctactcttccatgggagaagatgggctcctacactcattttatttccgacgatttcctgcgccgaggcggagatcccaacatcgtacattttgatagacagcctaagaaaggcaaagtatcaaaaaaatcccagcggataaagcggtccgattctcaatgggtcgggcggatgagaccgaggcttagtcaggaaaccgcaaaagcaaggatggaggcagactgggctgcccaaaacgagaacgaagaatataagaggctggctcgcagtaaacaggagctggctcgctggtgtgttaatactctgttgcagaatacacgatgcatcacccagtgtgatgaaattgtcgtcgtcatagaggacctcaatgttaaatccctgcacggtaaaggggctagagagcctggctgggacaattttttcactcccaaaacagagaatagatggttcattcagattctccataagacattttcagaactgcctaaacacaggggcgaacacgtcatcgaaggttgtccccttcggaccagtatcacttgtcccgcatgttcttattgtgataagaactcacgcaatggtgaaaaatttgtgtgcgtcgcttgcggggctaccttccacgcagatttcgaggtcgcaacctacaaccttgtacgccttgccacgaccggaatgcctatgccgaagagtttggagcggcaaggcggaggagagaaagccggcggcgcccgaaaagcccgaaagaaagcaaagcaggtcgaaaagattgtggtccaagcgaacgctaacgttactatgaatggtgccagtcttcattccccatag

SEQ ID NO: 30 - DNA, 2439 bp, Artificial
  Human optimized
  misc_feature (1)..(2439): cas_phi-10
atggacatgctcgatacggagacaaattacgcgacagagaccccagcccaacaacaagattacagcccgaaacccccgaagaaagcccagagagccccaaagggatttagtaagaaggcccggcctgaaaaaaaacccccgaaacctatcactttgttcacacaaaaacatttttccggtgtccggttccttaaacgggttattcgggatgcttcaaaaatactgaaactgagcgagagtcgaacgattacgtttctcgaacaggccattgagcgagatggctctgccccccccgatgtgactccaccggttcataatacgattatggccgtaacacgccctttcgaagagtggcccgaagttatcctcagcaaggctcttcaaaagcactgttacgcccttacaaagaaaataaagataaagacttggccgaaaaaaggacctggcaaaaagtgtcttgctgcatggtccgcgagaacaaaaatcccgctgatcccaggacaagtgcaagcaaccaacggactgttcgaccggatcgggagcatttatgacggcgtggagaaaaaggtaaccaaccgaaatgcgaacaaaaagttggagtacgatgaggctatcaaagagggaaggaatcccgcggttcctgagtacgaaacagcgtacaacatagacggcacattgattaataagccgggatacaatcccaacctctacatcacccagagtcgaacaccgcgactcattacagaggcagatagacccctcgttgaaaagatcctgtggcaaatggtagagaagaagacgcagagccgaaaccaggctcgcagagctaggctcgagaaagccgcgcaccttcagggccttcctgtcccaaaatttgtgcctgaaaaggtagatcgctcacagaaaatcgagatccggataatcgaccccctcgacaaaatcgaaccatatatgccccaggaccggatggcaataaaggcgagtcaggatgggcacgtaccatactggcaacgcccatttctctcaaagaggagaaatagaagagtaagagcaggttgggggaaacaagtgtcatctatccaggcgtggctcactggcgctctgcttgtcatagtacggctcggtaatgaagcattcctggccgatatccgaggagcattgaggaacgcacagtggaggaagctcctcaagccggatgccacttaccagtcactgtttaacctctttacgggagaccctgtcgtaaacacgaggacgaaccacctgaccatggcctaccgggagggtgtcgtgaatatcgtcaagagccgctcttttaagggcagacagaccagagaacaccttctcaccttgctgggtcaagggaaaacggtggcaggcgtctcctttgatcttgggcagaagcatgcggcgggattgttggcggctcattttggcctgggggaagatggtaatcccgtattcactccgatccaagcgtgtttccttccacaaaggtacttggacagtttgaccaactataggaataggtatgacgcgctcacattggatatgcgacgccaaagtctcctggcgctcacaccagctcaacaacaagaatttgcagacgcgcaacgcgacccaggggggcaggcgaagcgcgcatgctgtcttaaacttaatttgaatcccgatgaaataagatgggatcttgttagcggaatctcaaccatgataagcgatctttacatcgagaggggtggggatcctcgagatgtgcatcaacaggtggagaccaagccgaaaggtaaacgaaaatcagaaattcgaatccttaagattcgagatggaaagtgggcttatgacttccggcccaaaattgccgacgagactagaaaagcacagcgagagcagctctggaagctgcagaaagccagcagtgagttcgagcgcttgagtcggtacaaaattaacatagccagagcaatcgccaactgggccctccagtgggggcgagagcttagcggttgtgacattgtgatacccgtgctcgaagacctcaacgtcggttccaaatttttcgacggtaagggcaaatggctgctcggctgggataatcggttcactccaaaaaaggagaatagatggtttataaaagtgcttcataaggctgttgccgagttggctccccatcgcggggtcccggtctacgaggttatgccacaccgcacgagcatgacttgccccgcatgccactattgtcaccccacaaatagggaaggggatcgcttcgaatgtcaatcatgtcatgtagtcaagaatacagatagggacgtcgcaccttataatatacttcgcgtggcggtggaaggcaagacgctggaccgatggcaggccgagaagaaacctcaagcggaaccggataggccaatgatactcattgacaatcaggaatcttga

SEQ ID NO: 31 - protein, 726 aa, Artificial
  Mutant Cas_phi-3
  misc_feature (1)..(726): cas_phi-3 C-terminal truncation
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPM

SEQ ID NO: 32 - DNA, 2178 bp, Artificial
  Bacteria optimized
  misc_feature (1)..(2178): cas_phi-3 C-terminal truncation
atggaaaaagaaattaccgaactgaccaaaattcgccgcgaatttccgaacaaaaaatttagcagcaccgatatgaaaaaagcgggcaaactgctgaaagcggaaggcccggatgcggtgcgcgattttctgaacagctgccaggaaattattggcgattttaaaccgccggtgaaaaccaacattgtgagcattagccgcccgtttgaagaatggccggtgagcatggtgggccgcgcgattcaggaatattattttagcctgaccaaagaagaactggaaagcgtgcatccgggcaccagcagcgaagatcataaaagcttttttaacattaccggcctgagcaactataactataccagcgtgcagggcctgaacctgatttttaaaaacgcgaaagcgatttatgatggcaccctggtgaaagcgaacaacaaaaacaaaaaactggaaaaaaaatttaacgaaattaaccataaacgcagcctggaaggcctgccgattattaccccggattttgaagaaccgtttgatgaaaacggccatctgaacaacccgccgggcattaaccgcaacatttatggctatcagggctgcgcggcgaaagtgtttgtgccgagcaaacataaaatggtgagcctgccgaaagaatatgaaggctataaccgcgatccgaacctgagcctggcgggctttcgcaaccgcctggaaattccggaaggcgaaccgggccatgtgccgtggtttcagcgcatggatattccggaaggccagattggccatgtgaacaaaattcagcgctttaactttgtgcatggcaaaaacagcggcaaagtgaaatttagcgataaaaccggccgcgtgaaacgctatcatcatagcaaatataaagatgcgaccaaaccgtataaatttctggaagaaagcaaaaaagtgagcgcgctggatagcattctggcgattattaccattggcgatgattgggtggtgtttgatattcgcggcctgtatcgcaacgtgttttatcgcgaactggcgcagaaaggcctgaccgcggtgcagctgctggatctgtttaccggcgatccggtgattgatccgaaaaaaggcgtggtgacctttagctataaagaaggcgtggtgccggtgtttagccagaaaattgtgccgcgctttaaaagccgcgataccctggaaaaactgaccagccagggcccggtggcgctgctgagcgtggatctgggccagaacgaaccggtggcggcgcgcgtgtgcagcctgaaaaacattaacgataaaattaccctggataacagctgccgcattagctttctggatgattataaaaaacagattaaagattatcgcgatagcctggatgaactggaaattaaaattcgcctggaagcgattaacagcctggaaaccaaccagcaggtggaaattcgcgatctggatgtgtttagcgcggatcgcgcgaaagcgaacaccgtggatatgtttgatattgatccgaacctgattagctgggatagcatgagcgatgcgcgcgtgagcacccagattagcgatctgtatctgaaaaacggcggcgatgaaagccgcgtgtattttgaaattaacaacaaacgcattaaacgcagcgattataacattagccagctggtgcgcccgaaactgagcgatagcacccgcaaaaacctgaacgatagcatttggaaactgaaacgcaccagcgaagaatatctgaaactgagcaaacgcaaactggaactgagccgcgcggtggtgaactataccattcgccagagcaaactgctgagcggcattaacgatattgtgattattctggaagatctggatgtgaaaaaaaaatttaacggccgcggcattcgcgatattggctgggataacttttttagcagccgcaaagaaaaccgctggtttattccggcgtttcataaagcgtttagcgaactgagcagcaaccgcggcctgtgcgtgattgaagtgaacccggcgtggaccagcgcgacctgcccggattgcggcttttgcagcaaagaaaaccgcgatggcattaactttacctgccgcaaatgcggcgtgagctatcatgcggatattgatgtggcgaccctgaacattgcgcgcgtggcggtgctgggcaaaccgatg

SEQ ID NO: 33 - DNA, 2178 bp, Artificial
  Human optimized
  misc_feature (1)..(2178): cas_phi-3 C-terminal truncation
atggaaaaggagatcacagaactgaccaagatacgcagggagtttcccaacaaaaagttcagctcaacagatatgaaaaaagctggtaagcttctgaaagcggaagggcctgatgctgtacgcgatttcttgaattcatgtcaagagatcataggtgatttcaaaccgcctgtcaagacaaatattgtgagtattagtcgcccattcgaggaatggccagtcagtatggtcggtcgagccatccaagagtattacttctctttgacaaaagaggaactcgaaagcgtgcatccaggcacgagcagcgaagaccataagtccttcttcaatattacaggactgtctaactacaactatacatctgtgcagggattgaatctgatattcaaaaacgcaaaagccatctatgacggcacccttgtaaaggccaacaacaaaaacaagaagcttgaaaagaagtttaacgaaatcaaccacaaaaggtcacttgaaggtcttccaattataactccggacttcgaagagccttttgatgaaaatggacacctcaacaatccgccagggataaaccgaaatatatacgggtaccagggatgtgcagctaaggtctttgtgccgagtaagcataagatggtctccttgcctaaggagtacgaaggttacaatcgcgatccgaatctcagtcttgccggttttaggaaccggctggaaattcctgagggcgagcccgggcacgttccatggtttcagcgaatggacattcctgagggacaaatcggacacgtaaataagattcagagatttaacttcgtccatggcaaaaatagcgggaaggtcaagttcagcgacaagacagggagggttaagagatatcaccatagcaaatacaaagatgccacgaaaccatacaaattcctggaggaaagtaagaaagtctccgcgctggactcaatcctggcaatcattacgataggagacgactgggtggttttcgatatacgggggttgtatcggaacgtgttttatcgcgaattggcacaaaaaggtctcacagcagtacaattgctggatctgttcacgggtgaccctgtcatagatcctaaaaagggagtcgtgactttcagctacaaagagggagtagtcccagtgtttagccaaaagatcgttccacgatttaagtcaagagacacattggagaaactgacctctcaaggtcctgtggcgctcctgtctgttgatctgggacagaacgagcccgtagcagccagagtttgttcacttaaaaatataaacgacaaaattacgctcgacaatagctgtcgaatcagtttcctggacgactacaagaaacagataaaggactacagagacagtcttgacgaactggaaattaagatccgcctggaggccattaactccctcgagacaaaccaacaagttgaaattcgggacctcgacgtattttctgcggacagagcaaaagccaatacagtcgacatgttcgatatagaccctaatttgatatcctgggattctatgtccgacgcgcgggtatccacgcaaatctctgatctttaccttaagaacggaggcgatgagagtagggtttatttcgaaatcaataataagcgaataaaacgctccgattacaacattagtcaactggtgaggcccaagctgagcgactcaacccggaagaacttgaacgatagcatctggaagctcaaacgaactagtgaggagtacttgaaactgtcaaaacggaaactggagcttagtcgggccgtggtgaactatacgatcaggcagtcaaagcttctgagtggaattaacgatatcgtcataatattggaagatttggacgtaaaaaaaaagttcaacgggagaggcattcgcgatatcggttgggacaactttttctctagtaggaaggaaaaccggtggtttattcctgctttccataaagctttctccgagttgtcatcaaataggggcttgtgtgtgattgaggttaatccggcgtggacatcagcgacctgtccggattgtgggttttgctccaaggaaaaccgagatgggatcaactttacctgccgaaagtgtggagtgtcctatcatgctgacatagacgtagctaccctcaatatcgcgagggttgcggtgcttgggaagccaatg

SEQ ID NO: 34 - RNA, 25 nt, Artificial
  crRNA constant region
  misc_feature (1)..(25): cas_phi-1 crRNA constant region
aaacgauugcucgauuagucgagac

SEQ ID NO: 35 - RNA, 25 nt, Artificial
  crRNA constant region
  misc_feature (1)..(25): cas_phi-2 crRNA constant region
caacgauugccccucacgaggggac

SEQ ID NO: 36 - RNA, 25 nt, Artificial
  crRNA constant region
  misc_feature (1)..(25): cas_phi-3 crRNA constant region
uauugauugcccaguacgcugggac

SEQ ID NO: 37 - RNA, 45 nt, Artificial
  crRNA
  misc_feature (1)..(45): Example crRNA
uauugauugcccaguacgcugggacagcugguaaugggauaccuu

SEQ ID NO: 38 - DNA, 37 bp, Artificial
  Template strand
  misc_feature (1)..(37): Example template strand sequence
gctgtagcacaaggtatcccattaccagctgaattac

SEQ ID NO: 39 - DNA, 37 bp, Artificial
  Non template strand
  misc_feature (1)..(37): Example non-template strand sequence
gtaattcagctggtaatgggataccttgtgctacagc

(19) United States
(12) Patent Application Publication
(10) Pub. No.: US 2026/0234700 A1
(43) Pub. Date: Aug. 13, 2026

(54) MUTANT CAS12J ENDONUCLEASES

(71) Applicant: University of Copenhagen, Copenhagen K (DK)

(72) Inventors: Guillermo Montoya, Frederiksberg (DK); Arturo Carabias del Rey, Frederiksberg (DK); Anders Fuglsang, Vanløse (DK); Stefano Stella, Tivoli (RM) (IT)

(21) Appl. No.: 18/564,637

(22) PCT Filed: Jun. 2, 2022
(86) PCT No.: PCT/EP2022/065060

§ 371 Date: Nov. 28, 2023

(30) Foreign Application Priority Data
Jun. 2, 2021 (EP) 21177411.2

(51) Int. Cl. C12Q 1/6823; C12N 9/22; C12N 15/11; C12N 15/90

(52) U.S. Cl. CPC C12Q 1/6823; C12N 9/226; C12N 15/11; C12N 15/907; C12Y 301/21; C12N 2310/20

(57) ABSTRACT

The present invention relates to mutant Cas12j (also known as CasΦ) endonucleases having altered activity or improved properties compared to the corresponding wild type Cas12j endonuclease, as well as methods using the mutant Cas12j endonucleases.

### CROSS REFERENCE TO RELATED APPLICATIONS

This application is a U.S. National Stage of international patent application number PCT/EP2022/065060 filed Jun. 2, 2022, and which depends from and claims priority to European patent application number 21177411.2 filed Jun. 2, 2021, the entire contents of which are incorporated herein by reference.

### TECHNICAL FIELD

The present invention relates to mutant Cas12j (also known as CasΦ) endonucleases having altered activity or improved properties compared to the corresponding wild type Cas12j endonuclease. Methods for detection and quantification of a nucleic acid sequence, as well as methods for diagnosis of a disease are also disclosed.

### Reference to Sequence Listing Submitted Electronically

The instant application contains a Sequence Listing which has been submitted electronically in .txt format and is hereby incorporated by reference in its entirety. Said .txt copy, created Jul. 1, 2024, is named “2025 Mar. 5 Sequence Listing 16COPE-HO86502PA.txt” and is 139,336 bytes in size.

### BACKGROUND

Competition between microbes and their invaders has driven the evolution of a wide catalogue of defence systems to prevent the attack of mobile genetic elements (MGEs). Among them, CRISPR constitutes a type of adaptive immunity achieved by CRISPR-associated nucleases (Cas) and CRISPR RNAs (crRNAs) that assemble effector ribonucleoprotein complexes, which are guided by the crRNA to recognise and cleave complementary DNA (or RNA) for interference. CRISPR-Cas nucleases have been extensively used as tools for genome editing. The redesign of their guide RNA to target specific DNA sites, as well as the manipulation of the protein scaffold has provided a powerful method for genome modification in biomedical and biotechnological applications.

Although ubiquitously diversified among prokaryotes, CRISPR systems were also identified in the genome of bacteriophages. Recently, a new Class 2 family of CRISPR nucleases named Caso proteins, also known as Cas12j, were found in the biggiephage clade of “huge” phages. CasΦ proteins share a sequence identity lower than 7% with other CRISPR nucleases and display sequence and structural homology only in their RuvC domain with Class 2 type V members. CasΦ RNPs generate a staggered DNA double strand break (DSB) and unleash unspecific ssDNA cleavage after activation with a ssDNA molecule complementary to the crRNA, as other members of the Class 2 type V nucleases. In addition, the RuvC catalytic site of CasΦ1 and 2 also processes the precursor crRNA (pre-crRNA). CasΦ endonucleases recognise protospacers with a minimal T-rich PAM, and their small size (700-800 residues) together with the lack of a trans activation crRNA (tracrRNA) to build the functional RNP, make Caso a unique family of miniaturized RNA-guided nucleases.

CRISPR-Cas effector complexes are harnessed in vitro and in vivo for genome editing approaches, but specially the latter is limited by delivery problems, which is one of the main unmet needs in the field. Adeno-associated viral vectors (AAV) are commonly used for gene delivery. Yet, packaging of the genes coding for CRISPR-Cas effector complexes into an AAV vector is challenging due to its limited capacity, thus leaving little space for the insertion of additional regulatory elements. Recently, CasΦ enzymes have been shown to mediate genome editing in mammalian and plant cells2 expanding our repertoire of genome manipulation tools. The small size CasΦ RNPs can improve our genome editing approaches by alleviating the packing problems in the AAV vectors used for delivery.

However, questions regarding the detailed molecular mechanism of target DNA recognition, unzipping and subsequent cleavage by Cas nucleases remain unanswered, as no structural information is available. These CasΦ nucleases endonucleases are so far limited to being used in the same way as they act in nature, i.e. with the same requirements for specific target sequences, the same pattern and specific of cleavage etc. There is thus a need to discover the full potential of these enzymes and optimize them for use in known as well as new applications.

### SUMMARY

The present disclosure relates to mutant Cas12j endonucleases, such as mutant CasΦ-3 nucleases, that are capable of introducing single strand breaks or double strand breaks in nucleic acid target sequences which are either single stranded or double stranded. Furthermore, mutant Cas12j endonucleases of the present disclosure are able to bind nucleic acid targets that are either single stranded or double stranded without cutting said nucleic acid.

The new mutant Cas12j endonucleases disclosed herein present several advantages over wild type Cas12j endonucleases, such as a higher degree of miniaturization, altered PAM sequence requirements, or an improved specificity and/or enzymatic activity, and they can be favourably used for detection and quantification of target nucleic acid sequences.

Finally, the new mutant Cas12j endonucleases disclosed herein may also be used for diagnosis of a disease, such as by detection of genetic material deriving from an infectious agent causing the disease.

In some aspects, the present disclosure thus provides a mutant Cas12j endonuclease such as a mutant CasΦ-3 or an orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to:
i) the sequence corresponding to residues 1 to 20, 36 to 97, 104 to 119, 151 to 179, 204 to 379, 396 to 619, 651 to 679, and 701 to 726 of SEQ ID NO: 3, wherein said polypeptide sequence further comprises:
   a. at least one amino acid mutation in a first region of the NPID domain corresponding to residues 21 to 35 of SEQ ID NO: 3, wherein each mutation independently is an amino acid substitution, insertion or deletion; and/or
   b. at least one amino acid mutation in a first region of the TPID domain corresponding to residues 98 to 103 of SEQ ID NO: 3, wherein each mutation independently is an amino acid substitution, insertion or deletion; and/or
   c. at least one amino acid mutation in a second region of the TPID domain corresponding to residues 120 to 150 of SEQ ID NO: 3, wherein each mutation independently is an amino acid substitution, insertion or deletion; and/or
   d. at least one amino acid mutation in a third region of the TPID domain or in a first region of the RBD domain corresponding to residues 180 to 203 of SEQ ID NO: 3, wherein each mutation independently is an amino acid substitution, insertion or deletion; and/or
   e. at least one amino acid mutation in a second region of the RBD domain or in a first region of the RuvC-I domain corresponding to residues 380 to 395 of SEQ ID NO: 3, wherein each mutation independently is an amino acid substitution, insertion or deletion; and/or
   f. at least one amino acid mutation in a first region of the RuvC-II domain corresponding to residues 620 to 650 of SEQ ID NO: 3, wherein each mutation independently is an amino acid substitution, insertion or deletion; and/or
   g. at least one amino acid mutation in a second region of the RuvC-II domain corresponding to residues 680 to 700 of SEQ ID NO: 3, wherein each mutation independently is an amino acid substitution, insertion or deletion; and/or
   h. at least one amino acid mutation in a third region of the RuvC-II domain corresponding to residues 726 to 766 of SEQ ID NO: 3, wherein each mutation independently is an amino acid substitution, insertion or deletion;
and/or
ii) SEQ ID NO: 3, wherein said polypeptide sequence comprises at least one amino acid substitution in a position selected from the positions corresponding to residues 26, 30, 54, 55, 123, 197, 355, 360, 413, 618, 625, 626, 630, 643, 673, 675, 676, 680, 683, 691, 698, 701 and 708 of SEQ ID NO: 3.

In some aspects is provided a polynucleotide encoding the mutant Cas12j endonuclease or orthologue thereof as described herein.

In some aspects, the present disclosure provides a recombinant vector comprising a polynucleotide or a nucleic acid sequence encoding a mutant Cas12j endonuclease or orthologue thereof as defined above. In some embodiments, said polynucleotide or nucleic acid sequence is operably linked to a promoter.

In some aspects, the present disclosure thus provides a cell capable of expressing the mutant Cas12j endonuclease or orthologue thereof as disclosed herein, the polynucleotide as disclosed herein, or the recombinant vector according as disclosed herein.

In some aspects, the present disclosure provides a system for expression of a crRNA-Cas12j complex comprising
a. a polynucleotide as disclosed herein, or a recombinant vector according as disclosed herein comprising a polynucleotide encoding a mutant Cas12j endonuclease or orthologue thereof; and
b. a polynucleotide or a recombinant vector comprising a polynucleotide encoding a guide RNA (crRNA), optionally operably linked to a promoter.

In some aspects, the present disclosure provides a method of introducing a nucleic acid break in a first target nucleic acid, comprising the steps of:
a. designing a guide-RNA (crRNA) capable of recognising a second target nucleic acid comprising a protospacer adjacent motif (PAM);
b. contacting the crRNA of step a. with a mutant Cas12j endonuclease or orthologue thereof, wherein the mutant Cas12j endonuclease or orthologue thereof is as disclosed herein, or encoded by a polynucleotide or a vector as disclosed herein, thereby obtaining a crRNA-Cas12j complex capable of binding to said second target nucleic acid, and
c. contacting the crRNA and the mutant Cas12j endonuclease with said first target nucleic acid,

thereby introducing one or more nucleic acid breaks in the first target nucleic acid.

In some aspects, the present disclosure provides the use of a crRNA-Cas12j complex in a method for introducing a nucleic acid break in a first target nucleic acid, wherein:
a. a mutant Cas12j endonuclease or orthologue thereof is contacted with a guide RNA (crRNA), thereby obtaining a crRNA-Cas12j complex capable of recognizing a second target nucleic acid, the second target nucleic acid comprising a protospacer adjacent motif (PAM), and wherein the Cas12j endonuclease or orthologue thereof is according to any one of claim 1 to Error! Reference source not found.;
b. the crRNA-Cas12j complex is contacted with the first target nucleic acid;

whereby a nucleic acid break is made in the first target nucleic acid sequence.

In some aspects is provided an in vitro method of introducing a site-specific, double-stranded break at a second target nucleic acid in a mammalian cell, the method comprising introducing into the mammalian cell a crRNA-Cas12j complex, wherein the Cas12j is a mutant Cas12j endonuclease or orthologue as disclosed herein, and wherein the crRNA is specific for the second target nucleic acid.

In some aspects is provided a method for detection of a second target nucleic acid in a sample, the method comprising:
a. Providing a crRNA-Cas12j complex, wherein the Cas12j is a mutant Cas12j endonuclease or orthologue thereof as disclosed herein, and wherein the crRNA is specific for the second target nucleic acid;
b. Providing a labelled ssDNA, wherein the ssDNA is labelled with at least one set of interactive labels comprising at least one dye and at least one quencher;
c. Contacting the crRNA-Cas12j complex and the ssDNA with the sample, wherein the sample comprises at least one second target nucleic acid; and
d. Detecting cleavage of the ssDNA by detecting a fluorescent signal from the fluorophore,

thereby detecting the presence of the second target nucleic acid in the sample, wherein step c. optionally comprises activation of the crRNA-Cas12j complex.

In some aspects is also provided a method for detection and optionally quantification of a second target nucleic acid in a sample, the method comprising:
a. Providing a crRNA-Cas12j complex, wherein the Cas12j is a mutant Cas12j endonuclease or orthologue thereof as disclosed herein, wherein
   i. the mutant Cas12j has an abrogated endonuclease activity;
   ii. the mutant Cas12j comprises a detectable protein label; and
   iii, the crRNA is specific for the second target nucleic acid;
b. Contacting the crRNA-Cas12j complex with the sample, wherein the sample comprises at least one second target nucleic acid; and
c. Detecting and optionally quantifying the presence of the second target nucleic acid by detecting the protein label, such as a fluorescent signal.

In some aspects is provided an in vitro method for diagnosis of a disease in a subject, the method comprising:
a. Providing a crRNA-Cas12j complex, wherein the Cas12j is a mutant Cas12j endonuclease or orthologue thereof as disclosed herein, and wherein the crRNA is specific for a second target nucleic acid;
b. Providing a labelled ssDNA, wherein the ssDNA is labelled with at least one set of interactive labels comprising at least one dye and at least one quencher;
c. Providing a sample from the subject, wherein said sample comprises or is suspected of comprising the second target nucleic acid; and
d. Determining the level and/or concentration of the second target nucleic acid as defined in any one of the preceding claims,

wherein the second target nucleic acid is a nucleic acid fragment that correlates with the disease, such as wherein the second target nucleic acid is a biomarker of the disease,

thereby diagnosing a disease in a subject.

In some aspects is thus provided an in vitro method for diagnosis of an infectious disease in a subject, the method comprising:
a. Providing a crRNA-Cas12j complex, wherein the Cas12j is a mutant Cas12j endonuclease or orthologue thereof as disclosed herein, and wherein the crRNA is specific for a second target nucleic acid;
b. Providing a labelled ssDNA, wherein the ssDNA is labelled with at least one set of interactive labels comprising at least one dye and at least one quencher;
c. Providing a sample from the subject, wherein said sample comprises or is suspected of comprising the second target nucleic acid; and
d. Determining the level and/or concentration of the second target nucleic acid as defined in any one of the preceding claims,

wherein the second target nucleic acid is a nucleic acid of the genome of an infectious agent causing the disease or a fragment thereof,

thereby diagnosing an infectious disease in a subject.

### DESCRIPTION OF DRAWINGS

FIG. 1 shows the Cryo-EM structure of CasΦ3 endonuclease R-loop complex after target DNA cleavage. A) Domain architecture of CasΦ3 comprising the T-strand and NT-strand PAM interacting domains (TPID, NPID), the RNA-handle binding domain (RBD), the bridge helices (BH-I and BH-II), the RuvC domain including the insertion (amino acids 621-647) and the stop (STP) domain. B) Schematic diagram of the R-loop formed by the crRNA and the target DNA. Triangles represent phosphodiester cleavage positions in the T- and NT-strands; the light font nucleotides represent those not visualized in the structure. C) cryo-EM map of the CasΦ3/R-loop complex at 2.7 Å resolution. D) View of the R-loop structure and 2 nucleotides and the divalent metal ion in the catalytic site (polypeptide omitted for clarity). E) Overview of the CasΦ3-RNA-target-DNA ternary complex.

FIG. 2 shows CasΦ3 PAM recognition, uncoupling of the Watson-Crick dA-1:dT+1 pair and unzipping. A) Surface representation of CasΦ3-R-loop complex. The white dashed arrow shows the predicted path of the NT-strand to the DNA nuclease site after dG-2. B) Detailed view of the PAM nucleotides recognition and the dsDNA unwinding depicting the interactions of the conserved K26, K30, Q123 and Q197 residues. C) Zoom of the dT+1/dA−1 pair uncoupling, phosphate inversion and unzipping. Black dashed lines in b) and e) represent polar interactions between 2.2 and 3.2 Å. D) Representative dsDNA cleavage assays using CasΦ3 wild type (WT) and mutants. Oligonucleotides 3F-T-AAG-30 and 5F-NT-TTC-30 were used as substrate. T-strand (TS) and NT-strand (NTS) products are marked. Each experiment was repeated three to six times. E) Quantification of the activity based on the cleavage experiments as shown in d). Bars represent mean±s.d.

FIG. 3 shows assembly of the crRNA/DNA hybrid activates catalysis in the RuvC pocket. A) View of the hybrid showing the interaction of the crRNA with residues in the RuvC insertion. B) Inset depicting the hydrophobic interaction between the “plug” of the RuvC insertion and the and cavity of the STP domain. C) Representative dsDNA cleavage assays using CasΦ3 wild type (WT) and mutants. Oligonucleotides 3F-T-AAG-30 and 5F-NT-TTC-30 were used as substrate. T-strand (TS) and NT-strand (NTS) products are marked). Each experiment was repeated three times. D) Quantification of the activity based on the cleavage experiments as shown in c). Bars represent the mean±s.d. E) Detailed view of the RuvC catalytic site containing a dinucleotide and a divalent metal. The D708 side chain and the associated distances are shown for visualization purposes and. Black dashed lines in a) and e) represent polar interactions between 2.0 and 3.5 Å. (F-G) Trans ssDNA unspecific activity triggered by a target ssDNA oligo (F), or a dsDNA oligo (G). Marked with a dashed square, the mutants R643A and R643E do not compromise the specific dsDNA cleavage acitivity (C-D). However they abolish the unspecific trans ssDNA activity (F-G).

FIG. 4 shows a model of CasΦ3 PAM-dependent DNA recognition, unwinding and cleavage. This is a cartoon model depicting the stages of CasΦ3 nuclease staggered target DNA cleavage.

FIG. 5 shows CasΦ3 endonuclease biochemical characterisation. A) representative dsDNA cleavage pattern generated by CasΦ3 wild type (WT). T-strand (TS) and NT-strand (NTS) products are marked, showing a cut at position-13, -14 and -15 of the NT-strand, while the T-strand is cleaved at position +23. The sequence of the double labeled duplex is shown below, marking the position of the cut (triangles), and the size of the labelled products. B) Unspecific ssDNA degradation after activation with a specific target ssDNA of different length. C) Unspecific ssDNA degradation after activation with a specific dsDNA activator of different lengths. D) Schematic cartoon of the results shown in b) and c). Activation of the unspecific ssDNA cleavage is observed between 12-30 nt. (i) The RuvC domain of CasΦ3 RNP is inhibited. Full activation of the unspecific cleavage is observed when using a ssDNA or dsDNA activator pairing with the crRNA between 12-18 nt (ii and iv). The use of longer oligos as ssDNA (iii) or dsDNA (v) result in a reduction of the cleavage efficiency, likely due to a steric occlusion of the catalytic site by the T-strand and NT-strand. E) DNA cleavage dependency on divalent metal ions. Mg²⁺, Mn²⁺, Fe²⁺, Co²⁺ and Ni²⁺ metal ions support CasΦ3 catalytic activity, while Ca²⁺, Cu²⁺, Zn²⁺ do not. Depletion of the cation by EDTA abrogates phosphodiester hydrolysis. F) Cleavage assay using the target dsDNA shows the cleavage products of the different strands at different enzyme and substrates ratios. Quantification of the cleaved and non-cleaved dsDNA substrate is shown in the chart as mean±s.d. The curve shows an increase of the non-cleaved substrate when a 1:1 ratio is reached. An asymptotic behaviour is observed for the NT-strand products. G) Time course of the cleavage reaction by CasΦ3. CasΦ3 endonuclease completes the reaction in approximately 120 min for the T-strand while the NT-strand cleavage is completed in 20 min. H) Time course of the cleavage reaction by CasΦ3-ACT mutant lacking the C-terminal 39 residues. Experiments displayed are representative of at least three replicates.

FIG. 6 shows PAM specificity and crRNA/DNA hybrid assembly. A) cleavage assay with CasΦ3 WT and PAM interacting mutants, using target dsDNA as substrate containing different PAM or no PAM sequence. B) CasΦ3 activation of unspecific ssDNA degradation assay using an 18-nt dsDNA containing different PAM or no PAM sequence as activator. C) Unspecific ssDNA degradation by CasΦ3 WT and representative mutants involved in the PAM recognition (K30A/Q123A/Q197A), unwinding (K55A), and RuvC insertion (R643E) after activation with a 18-nt ssDNA without the PAM or a 18-nt dsDNA with the PAM. D) schematic representation explaining the results of the experiments shown in c). Gels shown are representative of three independent experiments.

### DETAILED DESCRIPTION

The invention is as defined in the claims.

The present disclosure relates to mutant Cas12j endonucleases or orthologues thereof and their uses. Throughout the present disclosure a “mutant Cas12j endonuclease” may be a naturally occurring mutant, for example a mutant encoded by a Cas12j gene carrying one or more single nucleotide polymorphisms (SNPs), or a non-naturally occurring mutant, for example a mutant obtained by direct mutagenesis or random mutagenesis of the Cas12j gene.

### Definitions

The term “codon” as used herein refers to a triplet of adjacent nucleotides coding for a specific amino acid.

The term “CRISPR-Cas system” as used herein refers to members of the CRISPR-Cas family. The prokaryotic adaptive immune system CRISPR-Cas (clustered regularly interspaced short palindromic repeats and CRISPR-associated proteins) can bind and cleave a target DNA sequence through RNA-guided recognition. According to their molecular architecture, the different members of the CRISPR-Cas system have been classified in two classes: class 1 encompasses several effector proteins, whereas class 2 systems use a single element (Makarova et al., 2015). Cas12j endonucleases have been described as a new member of class 2 type V CRISPR-Cas endonucleases present in a number of phage genomes (Pausch et al., 2020).

The term “endonuclease” as used herein refers to an enzyme capable of cleaving the phosphodiester bond within a polynucleotide chain. Some endonucleases are specific, i.e. they recognise a given nucleotide sequence which directs the site of cleavage. One example of endonucleases is nicking endonucleases. A nicking endonuclease as used herein is referred to an enzyme that cuts one strand of a double-stranded DNA to produce a “nicked” DNA molecule (“nickase” activity). A nicking endonuclease as used herein refers also to an endonuclease that cuts one strand of a single stranded DNA.

The term “fragment” as used herein indicates a non full-length part of a nucleic acid or polypeptide. Thus, a fragment is itself also a nucleic acid or polypeptide, respectively. DNA fragments are designated starting from the 5′-end throughout the present disclosure.

The term “gene editing” as used herein refers to the use of genetic engineering procedures to insert, delete or replace one or more nucleotides in a nucleotide sequence.

The term “guide RNA” will herein be used interchangeably with “crRNA” and refers to the RNA molecule which is required for recognition of a target nucleic acid sequence by CRISPR-Cas proteins, in particular a Cas12j endonuclease.

A homologue or functional homologue may be any polypeptide that exhibits at least some sequence identity with a reference polypeptide and has retained at least one aspect of the original functionality. Herein, a functional homologue of a Cas12j endonuclease is a polypeptide sharing at least some sequence identity with said Cas12j endonuclease or a fragment thereof which has the capability to function as an endonuclease similarly to said Cas12j endonuclease, i.e. it is capable of specifically binding a crRNA, and of specifically recognizing, binding and cleaving a target nucleic acid.

The term “protospacer adjacent motif (PAM)” as used herein refers to the DNA sequence immediately downstream the DNA sequence targeted by a CRISPR-Cas system such as a Cas12j endonuclease system. The crRNA of a crRNA-Cas12j complex is capable of recognizing and hybridizing only a target DNA sequence comprising a PAM.

The term “recognition” as used herein refers to the ability of a molecule to identify a nucleotide sequence. For example, an enzyme or a DNA binding domain may recognise a nucleic acid sequence as a potential substrate and bind to it. Preferably, the recognition is specific.

As used herein, the term “sequence identity” refers to two polynucleotide sequences that are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

As applied to polypeptides, peptides or proteins, a degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences.

The global percentage of sequence identity is determined with the algorithm GAP, BESTFIT, or FASTA in the Wisconsin Genetics Software Package Release 7.0, using default gap weights.

The terms “corresponding sequence”, “corresponding region” or “corresponding residue”, as is generally understood in the art, refers to a region or residue on a second amino acid or nucleotide sequence which occupies the same (i.e., equivalent) position as a region or residue on a first amino acid or nucleotide sequence, when the first and second sequences are optimally aligned for comparison purposes. Thus, a residue at a first position in a first peptide sequence does not necessarily correspond to a residue in said same first position in a second peptide sequence, but may instead correspond to a residue at a second position in the second peptide sequence that optimally aligns with the residue in said first position of said first peptide sequence, when the first and second peptide sequences are optimally aligned. Said alignment may be performed by any method known in the art, such as by using the Needleman-Wunsch algorithm (Needleman and Wunsch, 1970, J. Mo/. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later (available at https://www.ebi.ac.uk/Tools/psa/emboss_needle/). The parameters used may be gap open penalty of 10, gap extension penalty of 0.5, and the EBLOSUM62 (EMBOSS version of 30 BLOSUM62) substitution matrix.

The term “interactive labels” or “set of interactive labels” as used herein refers to at least one fluorophore and at least one quencher which can interact when they are located adjacently. When the interactive labels are located adjacently the quencher can quench the fluorophore signal. The interaction may be mediated by fluorescence resonance energy transfer (FRET).

The term “located adjacently” as used herein refers to the physical distance between two objects in close vicinity of one another. If a fluorophore and a quencher are located adjacently, the quencher is able to partly or fully quench the fluorophore signal. FRET quenching may typically occur over distances up to about 100 Å. Located adjacently as used herein may refer to distances below and/or around 100 Å.

The term “fluorescent label” or “fluorophore” as used herein refers to a fluorescent chemical compound that can re-emit light upon light excitation. The fluorophore absorbs light energy of a specific wavelength and re-emits light at a longer wavelength. The absorbed wavelengths, energy transfer efficiency, and time before emission depend on both the fluorophore structure and its chemical environment, as the molecule in its excited state interacts with surrounding molecules. Wavelengths of maximum absorption (~ excitation) and emission (for example, Absorption/Emission=485 nm/517 nm) are the typical terms used to refer to a given fluorophore, but the whole spectrum may be important to consider.

The term “quench” or “quenching” as used herein refers to any process which decreases the fluorescence intensity of a given substance such as a fluorophore.

Quenching may be mediated by fluorescence resonance energy transfer (FRET). FRET is based on classical dipole-dipole interactions between the transition dipoles of the donor (e.g. fluorophore) and acceptor (e.g. quencher) and is dependent on the donor-acceptor distance. FRET can typically occur over distances up to 100 Å. FRET also depends on the donor-acceptor spectral overlap and the relative orientation of the donor and acceptor transition dipole moments. Quenching of a fluorophore can also occur as a result of the formation of a non-fluorescent complex between a fluorophore and another fluorophore or non-fluorescent molecule. This mechanism is known as ‘contact quenching,’ ‘static quenching,’ or ‘ground-state complex formation

The term “quencher” as used herein refers to a chemical compound which is able to quench a given substance such as a fluorophore.

As used herein “the target strand” refers to the nucleic acid strand which interacts with the crRNA to form a crRNA-DNA hybrid. “The non-target strand” is complementary to the target strand.

The term “orthologue” as used herein refers to genes (and proteins encoded by said genes) inferred to be descended from the same ancestral sequence separated by a speciation event: when a species diverges into two separate species, the copies of a single gene in the two resulting species are said to be orthologous. Orthologs, or orthologous genes, are genes in different species that originated by vertical descent from a single gene of the last common ancestor. Cas12j orthologues can be identified and characterized based on sequence similarities to the present systems.

### Mutant Cas12j Endonucleases

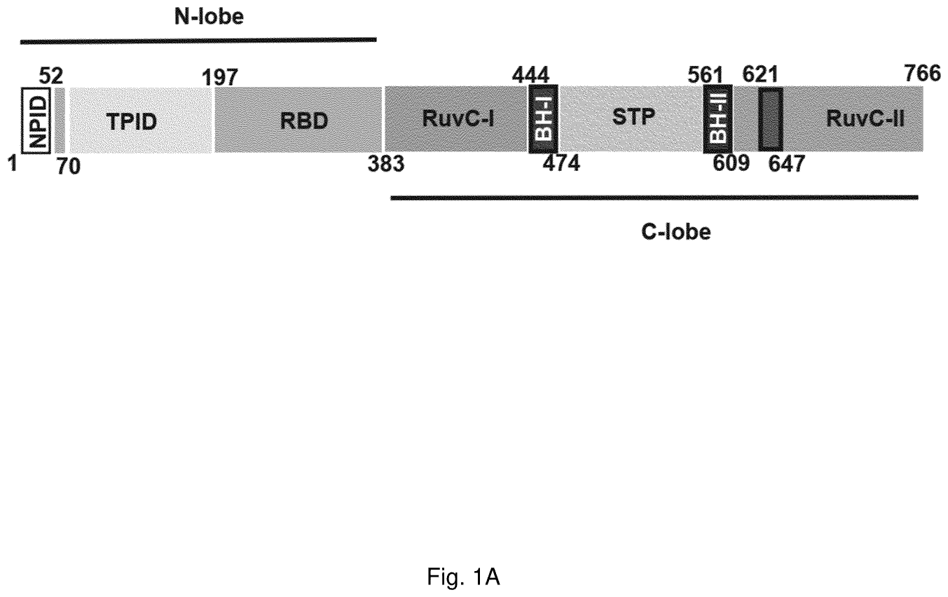

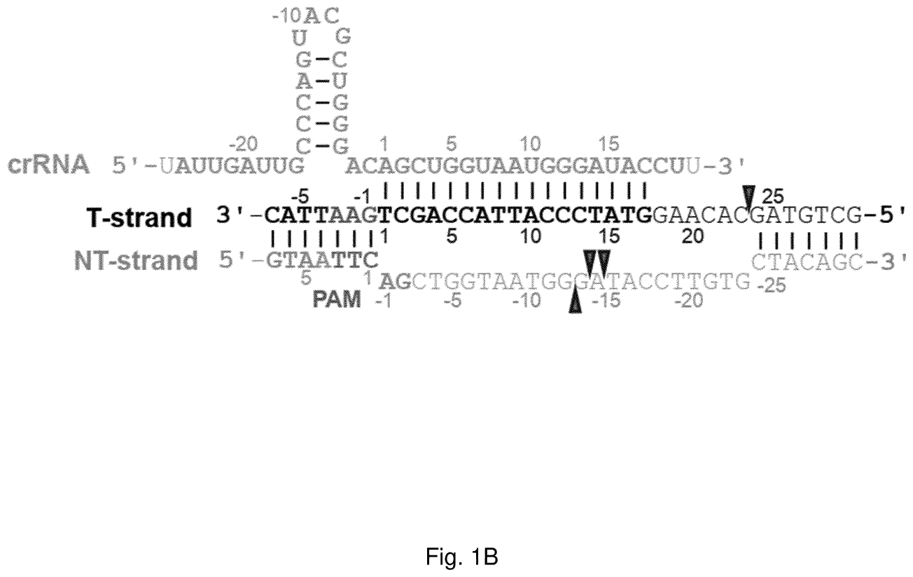

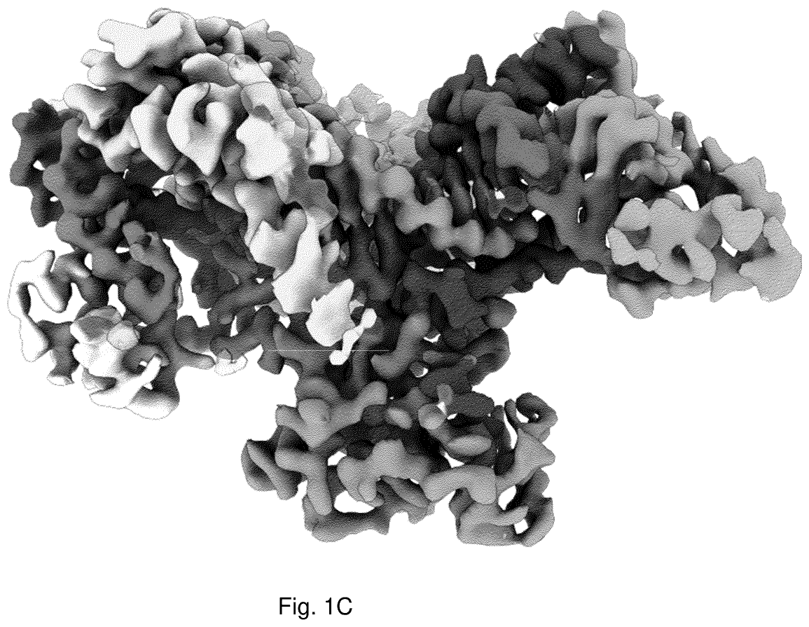

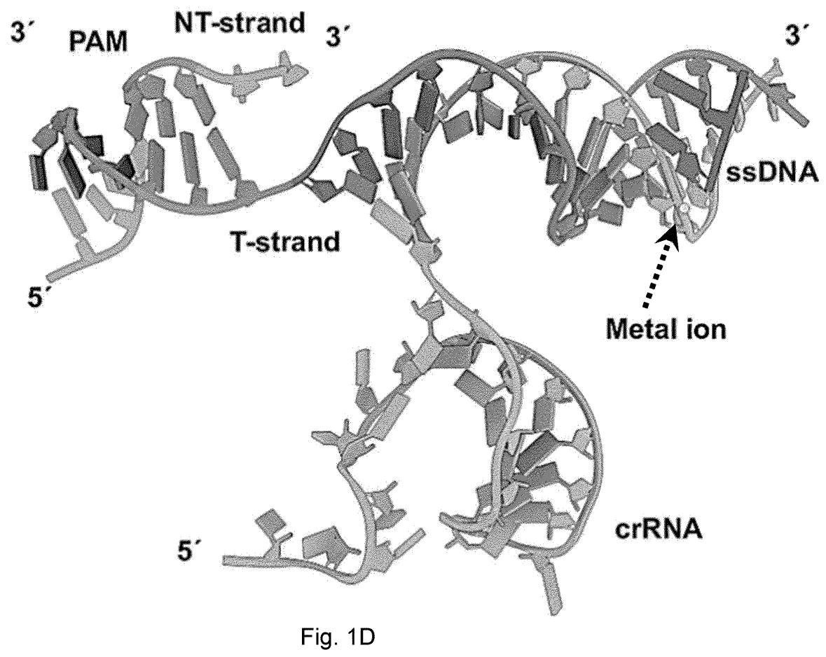

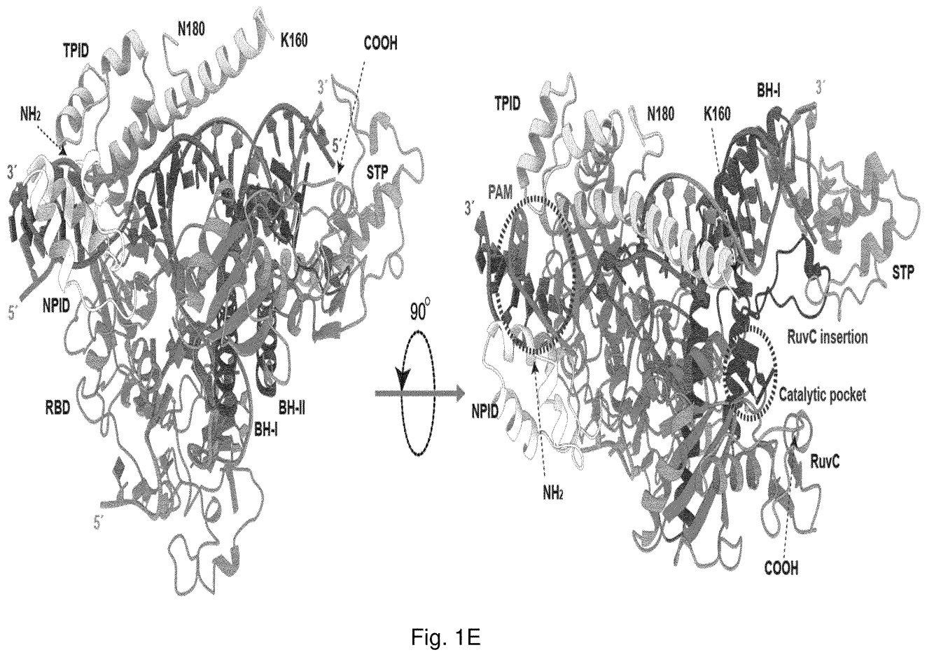

The inventors have identified and characterized several domains of the Casa12j family member CasΦ-3 (SEQ ID NO: 3), which are involved in different enzyme activities. FIG. 1A provides an overview of the domain organization of CasΦ-3 (SEQ ID NO: 3).

Using this information, the inventors have identified several key regions and key residues which when mutated improve or modify the enzyme activity of Cas12j endonuclease family members.

In particular, for CasΦ-3 (SEQ ID NO: 3), modifications of the following regions improve or modify the enzyme activity of the protein:
a first region of the NPID domain, said first region of the NPID domain defined residues 21 to 35 of SEQ ID NO: 3;
a first region of the TPID domain, said first region of the TPID domain defined as residues 98 to 103 of SEQ ID NO: 3;
a second region of the TPID domain, said second region of the TPID domain defined as residues 120 to 150 of SEQ ID NO: 3;
a third region of the TPID domain or a first region of the RBD domain, said third region of the TPID domain and said first region of the RBD domain defined as residues 180 to 203 of SEQ ID NO: 3;
a second region of the RBD domain or in a first region of the RuvC-I domain, said second region of the RBD domain and said first region of the RuvC-I domain defined as residues 380 to 395 of SEQ ID NO: 3;
a first region of the RuvC-II domain, said first region of the RuvC-II domain defined as residues 620 to 650 of SEQ ID NO: 3;
a second region of the RuvC-II domain, said second region of the RuvC-II domain defined as residues 680 to 700 of SEQ ID NO: 3;
a third region of the RuvC-II domain, said third region of the RuvC-II domain defined as residues 726 to 766 of SEQ ID NO: 3.

Substitution, insertion or deletion of amino acids in any of these regions may result in modified enzyme activity, as will be detailed herein below. Modifications of corresponding regions in other Cas12j family members than CasΦ-3 may provide similar improved or modified enzymatic activities.

In addition, key residues were identified which appear important for enzymatic activity, i.e. mutations or deletions of any of these residues also modifies enzyme activity. These residues are at positions 26, 30, 54, 55, 123, 197, 355, 360, 413, 618, 625, 626, 630, 643, 673, 675, 676, 680, 683, 691, 698, 701 and 708 of SEQ ID NO: 3 for CasΦ-3. Residues corresponding to these positions in other Cas12j family members may be similarly important for enzyme activity, i.e. mutations or deletions of any of these residues also modifies enzyme activity.

The present disclosure thus relates to modified Cas12j proteins having altered activities.

In some aspects, the present disclosure thus provides a mutant Cas12j endonuclease such as a mutant CasΦ-3 or an orthologue thereof comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to:
i) the sequence corresponding to residues 1 to 20, 36 to 97, 104 to 119, 151 to 179, 204 to 379, 396 to 619, 651 to 679, and 701 to 726 of SEQ ID NO: 3, wherein said polypeptide sequence further comprises:
   a. at least one amino acid mutation in a first region of the NPID domain corresponding to residues 21 to 35 of SEQ ID NO: 3, wherein each mutation independently is an amino acid substitution, insertion or deletion; and/or
   b. at least one amino acid mutation in a first region of the TPID domain corresponding to residues 98 to 103 of SEQ ID NO: 3, wherein each mutation independently is an amino acid substitution, insertion or deletion; and/or
   c. at least one amino acid mutation in a second region of the TPID domain corresponding to residues 120 to 150 of SEQ ID NO: 3, wherein each mutation independently is an amino acid substitution, insertion or deletion; and/or
   d. at least one amino acid mutation in a third region of the TPID domain or in a first region of the RBD domain corresponding to residues 180 to 203 of SEQ ID NO: 3, wherein each mutation independently is an amino acid substitution, insertion or deletion; and/or
   e. at least one amino acid mutation in a second region of the RBD domain or in a first region of the RuvC-I domain corresponding to residues 380 to 395 of SEQ ID NO: 3, wherein each mutation independently is an amino acid substitution, insertion or deletion; and/or
   f. at least one amino acid mutation in a first region of the RuvC-II domain corresponding to residues 620 to 650 of SEQ ID NO: 3, wherein each mutation independently is an amino acid substitution, insertion or deletion; and/or
   g. at least one amino acid mutation in a second region of the RuvC-II domain corresponding to residues 680 to 700 of SEQ ID NO: 3, wherein each mutation independently is an amino acid substitution, insertion or deletion; and/or
   h. at least one amino acid mutation in a third region of the RuvC-II domain corresponding to residues 726 to 766 of SEQ ID NO: 3, wherein each mutation independently is an amino acid substitution, insertion or deletion;
and/or
ii) SEQ ID NO: 3, wherein said polypeptide sequence comprises at least one amino acid substitution in a position selected from the positions corresponding to residues 26, 30, 54, 55, 123, 197, 355, 360, 413, 618, 625, 626, 630, 643, 673, 675, 676, 680, 683, 691, 698, 701 and 708 of SEQ ID NO: 3.

In some embodiments, the mutant Cas12j endonuclease is a mutant of a Cas12j endonuclease selected from the group consisting of CasΦ-1 (SEQ ID NO: 1), CasΦ-2 (SEQ ID NO: 2), CasΦ-3 (SEQ ID NO: 3), CasΦ-4 (SEQ ID NO: 4), CasΦ-5 (SEQ ID NO: 5), CasΦ-6 (SEQ ID NO: 6), CasΦ-7 (SEQ ID NO: 7), CasΦ-8 (SEQ ID NO: 8), CasΦ-9 (SEQ ID NO: 9), and CasΦ-10 (SEQ ID NO: 10). In some embodiments, the mutant Cas12j endonuclease is a mutant of CasΦ-1 (SEQ ID NO: 1). In some embodiments, the mutant Cas12j endonuclease is a mutant of CasΦ-2 (SEQ ID NO: 2). In some embodiments, the mutant Cas12j endonuclease is a mutant of CasΦ-3 (SEQ ID NO: 3). In some embodiments, the mutant Cas12j endonuclease is a mutant of CasΦ-4 (SEQ ID NO: 4). In some embodiments, the mutant Cas12j endonuclease is a mutant of CasΦ-5 (SEQ ID NO: 5). In some embodiments, the mutant Cas12j endonuclease is a mutant of CasΦ-6 (SEQ ID NO: 6). In some embodiments, the mutant Cas12j endonuclease is a mutant of CasΦ-7 (SEQ ID NO: 7). In some embodiments, the mutant Cas12j endonuclease is a mutant of CasΦ-8 (SEQ ID NO: 8). In some embodiments, the mutant Cas12j endonuclease is a mutant of CasΦ-9 (SEQ ID NO: 9). In some embodiments, the mutant Cas12j endonuclease is a mutant of CasΦ-10 (SEQ ID NO: 10). In preferred embodiments, the mutant Cas12j endonuclease is a mutant CasΦ-1, such as a mutant CasΦ-2, or such as a mutant CasΦ-3.

In some embodiments, the mutant Cas12j endonuclease or orthologue thereof is derived from a Biggiephage. For example, the mutant Cas12j endonuclease may be derived from a phage with the NCBI genome/sample accession identifier ERS4026370, ERS4025728, ERS4026385, or ERS4025730.

The inventors have surprisingly found that a specific C-terminal truncation of the protein preserves the catalytic activity of the enzyme, enabling a further miniaturization of the protein.

In some embodiments is thus provided a mutant Cas12j endonuclease, such as a mutant Cas-3 or an orthologue thereof, comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to the sequence corresponding to residues 1 to 726 of SEQ ID NO: 3, wherein said polypeptide sequence further comprises a C-terminal deletion of the sequence corresponding to residues 727 to 766 of SEQ ID NO: 3. In some embodiments is thus provided a mutant Cas12j endonuclease, such as a mutant CasΦ-3 or an orthologue thereof, comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to SEQ ID NO: 31.

In some embodiments is provided a mutant Cas12j endonuclease, such as a mutant CasΦ-3 or an orthologue thereof, comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to the sequence corresponding to residues 1 to 20 and 36 to 726 of SEQ ID NO: 3, wherein said polypeptide sequence further comprises at least one amino acid mutation in a first region of the NPID domain corresponding to residues 21 to 35 of SEQ ID NO: 3, wherein each mutation independently is an amino acid substitution, insertion or deletion. The at least one amino acid substitution, insertion or deletion may be substitution, insertion or deletion of at least 1, 2, 3, 4, 5, 6, 7, 8, 9, 10, 11, 12, 13, 14 or 15 contiguous or non-contiguous amino acids of said first region of the NPID domain.

In some embodiments is provided a mutant Cas12j endonuclease, such as a mutant CasΦ-3 or an orthologue thereof, comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to the sequence corresponding to residues 1 to 97 and 104 to 726 of SEQ ID NO: 3, wherein said polypeptide sequence further comprises at least one amino acid mutation in a first region of the TPID domain corresponding to residues 98 to 103 of SEQ ID NO: 3, wherein each mutation independently is an amino acid substitution, insertion or deletion. The at least one amino acid substitution, insertion or deletion may be substitution, insertion or deletion of at least 1, 2, 3, 4, 5, 6, 7, 8, 9, 10, 11, 12, 13, 14 or 15 contiguous or non-contiguous amino acids of said first region of the TPID domain.

In some embodiments is provided a mutant Cas12j endonuclease, such as a mutant CasΦ-3 or an orthologue thereof, comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to the sequence corresponding to residues 1 to 119 and 151 to 726 of SEQ ID NO: 3, wherein said polypeptide sequence further comprises at least one amino acid mutation in a second region of the TPID domain corresponding to residues 120 to 150 of SEQ ID NO: 3, wherein each mutation independently is an amino acid substitution, insertion or deletion. The at least one amino acid substitution, insertion or deletion may be substitution, insertion or deletion of at least 1, 2, 3, 4, 5, 6, 7, 8, 9, 10, 11, 12, 13, 14 or 15 contiguous or non-contiguous amino acids of said second region of the TPID domain.

In some embodiments is provided a mutant Cas12j endonuclease, such as a mutant CasΦ-3 or an orthologue thereof, comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to the sequence corresponding to residues 1 to 179 and 204 to 726 of SEQ ID NO: 3, wherein said polypeptide sequence further comprises at least one amino acid mutation in a third region of the TPID domain or in a first region of the RBD domain corresponding to residues 180 to 203 of SEQ ID NO: 3, wherein each mutation independently is an amino acid substitution, insertion or deletion. The at least one amino acid substitution, insertion or deletion may be substitution, insertion or deletion of at least 1, 2, 3, 4, 5, 6, 7, 8, 9, 10, 11, 12, 13, 14 or 15 contiguous or non-contiguous amino acids of said third region of the TPID domain and said first region of the RBD domain.

In some embodiments is provided a mutant Cas12j endonuclease, such as a mutant CasΦ-3 or an orthologue thereof, comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to the sequence corresponding to residues 1 to 379 and 396 to 726 of SEQ ID NO: 3, wherein said polypeptide sequence further comprises at least one amino acid mutation in a second region of the RBD domain or in a first region of the RuvC-I domain corresponding to residues 380 to 395 of SEQ ID NO: 3, wherein each mutation independently is an amino acid substitution, insertion or deletion. The at least one amino acid substitution, insertion or deletion may be substitution, insertion or deletion of at least 1, 2, 3, 4, 5, 6, 7, 8, 9, 10, 11, 12, 13, 14 or 15 contiguous or non-contiguous amino acids of said second region of the RBD domain and said first region of the RuvC-I domain.

In some embodiments is provided a mutant Cas12j endonuclease, such as a mutant CasΦ-3 or an orthologue thereof, comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to the sequence corresponding to residues 1 to 619 and 651 to 726 of SEQ ID NO: 3, wherein said polypeptide sequence further comprises at least one amino acid mutation in a first region of the RuvC-II domain corresponding to residues 620 to 650 of SEQ ID NO: 3, wherein each mutation independently is an amino acid substitution, insertion or deletion. The at least one amino acid substitution, insertion or deletion may be substitution, insertion or deletion of at least 1, 2, 3, 4, 5, 6, 7, 8, 9, 10, 11, 12, 13, 14 or 15 contiguous or non-contiguous amino acids of said first region of the RuvC-II domain.

In some embodiments is provided a mutant Cas12j endonuclease, such as a mutant CasΦ-3 or an orthologue thereof, comprising a polypeptide sequence having at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity to the sequence corresponding to residues 1 to 679 and 701 to 726 of SEQ ID NO: 3, wherein said polypeptide sequence further comprises at least one amino acid mutation in a second region of the RuvC-II domain corresponding to residues 680 to 700 of SEQ ID NO: 3, wherein each mutation independently is an amino acid substitution, insertion or deletion. The at least one amino acid substitution, insertion or deletion may be substitution, insertion or deletion of at least 1, 2, 3, 4, 5, 6, 7, 8, 9, 10, 11, 12, 13, 14 or 15 contiguous or non-contiguous amino acids of said second region of the RuvC-II domain.

In some embodiments, said region is substituted with another region, such as a corresponding region, of a different protein. Said domain substitution may provide additional functionality to the enzyme, e.g. such as substitution of the CasΦ-3 RuvC domain with the corresponding CasΦ-1 or CasΦ-2 RuvC domain providing CasΦ-3 the ability to process precursor crRNA (pre-crRNA). In some embodiments, said first region of the RuvC-I domain, said first region of the RuvC-II domain, and/or said second region of the RuvC-II domain of CasΦ-3 as described herein above is substituted with the corresponding region of CasΦ-1 or CasΦ-2. Examples of corresponding RuvC-I and RuvC-II domains are provided in Table 1 herein below.

The at least one substitution may be a substitution of at least at least 10 amino acid residues, such as at least 15, such as at least 25, such as least 50, such as at least 75, such as at least 100, such as at least 150, such as at least 200, such as at least 250, such as at least 300, such as at least 350, such as at least 400, such as at least 450, such as at least 500 amino acid residues. In some embodiments, the at least one substitution is in the range of 10 to 500 amino acid residues, such as in the range of 25 to 450 amino acid residues, such as in the range of 50 to 400 amino acid residues, such as in the range of 50 to 350 amino acid residues, such as in the range of 50 to 300 amino acid residues, such as in the range of 50 to 300 amino acid residues, such as in the range of 50 to 250 amino acid residues, such as in the range of 50 to 200 amino acid residues, such as in the range of 50 to 150 amino acid residues, or such as in the range of 75 to 150 amino acid residues.

It will be understood that the at least one amino acid substitution or deletion as defined above may refer to deletion of some amino acids in a domain, while other amino acids may be substituted.

All of the above mutants may comprise or further comprise at least one amino acid substitution and/or deletion in one or more of the residues corresponding to positions 26, 30, 54, 55, 123, 197, 355, 360, 413, 618, 625, 626, 630, 643, 673, 675, 676, 680, 683, 691, 698, 701 and 708 of SEQ ID NO: 3.

In some embodiments, the at least one amino acid substitution is a substitution of an amino acid having a charged side chain to an amino acid having an uncharged side chain.

In some embodiments, the at least one amino acid substitution is a substitution of an amino acid having a charged side chain to an amino acid residue having a non-polar side chain.

In some embodiments, the at least one amino acid substitution is a substitution of an amino acid having a charged side chain to a glycine, alanine, valine, leucine, isoleucine, serine or threonine.

In some embodiments, the at least one amino acid substitution is a substitution of an amino acid having a charged side chain to a glycine.

In some embodiments, the at least one amino acid substitution is a substitution of an amino acid to an alanine.

In some embodiments, the at least one amino acid substitution or deletion is a substitution or deletion of at least 2 residues, such as a substitution or deletion of at least 3 residues, such as a substitution or deletion of at least 4 residues, such as a substitution or deletion of at least 5 residues, such as a substitution or deletion of at least 6 residues, such as a substitution or deletion of at least 7 residues, such as a substitution or deletion of at least 8 residues, such as a substitution or deletion of at least 9 residues, such as a substitution or deletion of at least 10 residues, such as a substitution or deletion of at least 11 residues, such as a substitution or deletion of at least 12 residues, such as a substitution or deletion of at least 13 residues, such as a substitution or deletion of at least 14 residues, such as a substitution or deletion of at least 15 residues, such as a substitution or deletion of at least 20 residues, such as a substitution or deletion of at least 25 residues, such as a substitution or deletion of at least 30 residues, such as a substitution or deletion of at least 35 residues, or such as a substitution or deletion of at least 40 residues.

In some embodiments, the at least one amino acid substitution is in the NPID domain. In some embodiments, the at least one amino acid substitution is in the TPID domain. In some embodiments, the at least one amino acid substitution is in the RBD domain. In some embodiments, the at least one amino acid substitution is in the RuvC-I domain. In some embodiments, the at least one amino acid substitution is in the RuvC-II domain.

Examples of domain positions for Cas12j nucleases are provided in Table 1, below.

TABLE 1
Selected domains of Cas12j endonucleases.

| | Domain residues | | | | |
|---|---|---|---|---|---|
| Endonuclease | NPID | TPID | RBD | RuvC-I | RuvC-II |
| Casφ-1 | 1 to 54 | 72 to 199 | 199 to 341 | 341 to 399 | 570 to 707 |
| (SEQ ID NO: 1) | | | | | |
| Casφ-2 | 1 to 56 | 74 to 202 | 202 to 363 | 363 to 425 | 597 to 757 |
| (SEQ ID NO: 2) | | | | | |
| Casφ-3 | 1 to 52 | 70 to 197 | 197 to 383 | 383 to 444 | 609 to 766 |
| (SEQ ID NO: 3) | | | | | |
| Casφ-4 | 1 to 63 | 81 to 213 | 213 to 389 | 389 to 450 | 616 to 765 |
| (SEQ ID NO: 4) | | | | | |
| Casφ-5 | 1 to 59 | 77 to 207 | 207 to 384 | 384 to 445 | 622 to 793 |
| (SEQ ID NO: 5) | | | | | |
| Casφ-6 | 1 to 58 | 76 to 194 | 194 to 354 | 354 to 415 | n.a. |
| (SEQ ID NO: 6) | | | | | |
| Casφ-7 | 1 to 68 | 86 to 227 | 227 to 394 | 394 to 455 | 623 to 772 |
| (SEQ ID NO: 7) | | | | | |
| Casφ-8 | 1 to 51 | 70 to 195 | 195 to 339 | 339 to 400 | 562 to 717 |
| (SEQ ID NO: 8) | | | | | |
| Casφ-9 | 1 to 69 | 87 to 220 | 220 to 351 | 351 to 458 | 630 to 793 |
| (SEQ ID NO: 9) | | | | | |
| Casφ-10 | 1 to 98 | 116 to 240 | 240 to 433 | 433 to 496 | 669 to 812 |
| (SEQ ID NO: 10) | | | | | |

In some embodiments, the amino acid substitution in the RuvC-I and/or RuvC-II domain is the substitution of an amino acid that is not a glutamic acid or an aspartic acid.

In some embodiments, the mutant Cas12j endonuclease or orthologue thereof comprises a substitution at a position corresponding to K26 of SEQ ID NO: 3 or SEQ ID NO: 31. In some embodiments, the mutant Cas12j endonuclease or orthologue thereof comprises a substitution at a position corresponding to K30 of SEQ ID NO: 3 or SEQ ID NO: 31. In some embodiments, the mutant Cas12j endonuclease or orthologue thereof comprises a substitution at a position corresponding to F54 of SEQ ID NO: 3 or SEQ ID NO: 31. In some embodiments, the mutant Cas12j endonuclease or orthologue thereof comprises a substitution at a position corresponding to K55 of SEQ ID NO: 3 or SEQ ID NO: 31. In some embodiments, the mutant Cas12j endonuclease or orthologue thereof comprises a substitution at a position corresponding to Q123 of SEQ ID NO: 3 or SEQ ID NO: 31. In some embodiments, the mutant Cas12j endonuclease or orthologue thereof comprises a substitution at a position corresponding to Q197 of SEQ ID NO: 3 or SEQ ID NO: 31. In some embodiments, the mutant Cas12j endonuclease or orthologue thereof comprises a substitution at a position corresponding to L355 of SEQ ID NO: 3 or SEQ ID NO: 31. In some embodiments, the mutant Cas12j endonuclease or orthologue thereof comprises a substitution at a position corresponding to T360 of SEQ ID NO: 3 or SEQ ID NO: 31. In some embodiments, the mutant Cas12j endonuclease or orthologue thereof comprises a substitution at a position corresponding to D413 of SEQ ID NO: 3 or SEQ ID NO: 31. In some embodiments, the mutant Cas12j endonuclease or orthologue thereof comprises a substitution at a position corresponding to E618 of SEQ ID NO: 3 or SEQ ID NO: 31.

In some embodiments, the mutant Cas12j endonuclease or orthologue thereof comprises a substitution at a position corresponding to K625 of SEQ ID NO: 3 or SEQ ID NO: 31. In some embodiments, the mutant Cas12j endonuclease or orthologue thereof comprises a substitution at a position corresponding to F626 of SEQ ID NO: 3 or SEQ ID NO: 31. In some embodiments, the mutant Cas12j endonuclease or orthologue thereof comprises a substitution at a position corresponding to G630 of SEQ ID NO: 3 or SEQ ID NO: 31.

In some embodiments, the mutant Cas12j endonuclease or orthologue thereof comprises a substitution at a position corresponding to R643 of SEQ ID NO: 3 or SEQ ID NO: 31. In some embodiments, the mutant Cas12j endonuclease or orthologue thereof comprises a substitution at a position corresponding to R643 of SEQ ID NO: 3 (CasΦ-3) or SEQ ID NO: 31. In some embodiments, said substitution is an R643E substitution. Said R643E substitution may abrogate the unspecific endonuclease activity of the enzyme. Thus, in some embodiments, the specific double stranded DNA cleavage activity is unchanged while any unspecific single stranded DNA cleavage activity of the Cas12j endonuclease is abrogated. In some embodiments, said substitution is an R643A substitution. Said R643A substitution may abrogate the unspecific endonuclease activity of the enzyme. Thus in some embodiments, the specific double stranded DNA cleavage activity is unchanged while any unspecific single stranded DNA cleavage activity of the Cas12j endonuclease is abrogated.

In some embodiments, the mutant Cas12j endonuclease or orthologue thereof comprises a substitution at a position corresponding to P673 of SEQ ID NO: 3 or SEQ ID NO: 31. In some embodiments, the mutant Cas12j endonuclease or orthologue thereof comprises a substitution at a position corresponding to W675 of SEQ ID NO: 3 or SEQ ID NO: 31. In some embodiments, the mutant Cas12j endonuclease or orthologue thereof comprises a substitution at a position corresponding to T676 of SEQ ID NO: 3 or SEQ ID NO: 31. In some embodiments, the mutant Cas12j endonuclease or orthologue thereof comprises a substitution at a position corresponding to C680 of SEQ ID NO: 3 or SEQ ID NO: 31. In some embodiments, the mutant Cas12j endonuclease or orthologue thereof comprises a substitution at a position corresponding to C683 of SEQ ID NO: 3 or SEQ ID NO: 31.

In some embodiments, the mutant Cas12j endonuclease or orthologue thereof comprises a substitution at a position corresponding to R691 of SEQ ID NO: 3 (CasΦ-3) or SEQ ID NO: 31. In some embodiments, said substitution is an R691A substitution. Said R691A substitution may abrogate the endonuclease activity of the enzyme. In some embodiments the specific double stranded DNA cleavage activity and/or any unspecific single stranded DNA cleavage activity of the Cas12j endonuclease is abrogated. Thus, in some embodiments there is a total loss of specific double stranded DNA cleavage activity and/or any unspecific single stranded DNA cleavage activity of the Cas12j endonuclease. In some embodiments, said R691A substitution corresponds to an R651A substitution in CasΦ-1 (SEQ ID NO: 1). In some embodiments, said R691A substitution corresponds to an R678A substitution in CasΦ-2 (SEQ ID NO: 2).

In some embodiments, the mutant Φ12j endonuclease or orthologue thereof comprises a substitution at a position corresponding to C698 of SEQ ID NO: 3 or SEQ ID NO: 31.

In some embodiments, the mutant Cas12j endonuclease or orthologue thereof comprises a substitution at a position corresponding to C701 of SEQ ID NO: 3 or SEQ ID NO: 31.

In some embodiments, the mutant Cas12j endonuclease or orthologue thereof comprises a substitution at a position corresponding to D708 of SEQ ID NO: 3 or SEQ ID NO: 31.

In some embodiments, the mutant endonuclease is conjugated to a protein tag.

In some embodiments, the protein tag is a FLAG-tag. In some embodiments, the protein tag is a HA-tag. In some embodiments, the protein tag is a biotin. In some embodiments, the protein tag is a chitin binding protein (CBP). In some embodiments, the protein tag is a maltose binding protein (MBP). In some embodiments, the protein tag is a strep-tag. In some embodiments, the protein tag is a glutathione-S-transferase (GST). In some embodiments, the protein tag is a poly(His) tag. In some embodiments, the protein tag is an enzyme, such as peroxidase, a biotin ligase, or a base editing enzyme, such as a cytidine or adenine deaminase. In some embodiments, the protein tag is a transcriptional regulator, such as a transcription factor. In some embodiments, the protein tag is a fluorescent tag, such as GFP, Venus or fluorescein.

The mutants as disclosed herein comprising a conjugated protein tag are useful in a range of application, such as in base editing, epigenetic remodelling, transcriptional regulation, investigation of chromatin structure and detecting and quantification of target nucleic acid sequences.

The mutant Cas12j endonuclease or orthologue thereof as disclosed herein may have one or more improved and/or altered activities compared to the wild type endonuclease.

In some embodiments, said altered and/or improved activity is an improvement and/or an alteration in an enzyme activity related to double-stranded cleavage of a target nucleic acid sequence. In some embodiments, said altered and/or improved activity is an improvement and/or an alteration in an enzyme activity related to single-stranded cleavage of a target nucleic acid sequence. In some embodiments, said altered and/or improved activity is an improvement and/or an alteration in an enzyme activity related to target nucleic acid recognition.

In some embodiments, the altered activity is alteration in cleavage activity from inducing double-stranded nucleic acid breaks to inducing single-stranded nucleic acid breaks (nickase activity). Thus, in some embodiments, the mutant Cas12j endonuclease is a nicking endonuclease.

In some embodiments, said altered and/or improved activity is increased speed of catalysis.

In some embodiments, said altered activity is altered protospacer adjacent motif (PAM) sequence recognition. An altered PAM sequence recognition enables the targeting of nucleic sequences that could not be targeted with the unmodified enzyme.

In some embodiments, said altered and/or improved activity is altered length of an overhang produced resulting from a staggered nucleic acid double-strand break. In some embodiments, said altered and/or improved activity is thus an altered cleavage pattern.

In some embodiments, said altered and/or improved activity is decreased frequency of off-target cleavage.

In some embodiments, said altered activity is abrogation of nuclease activity. Thus, in some embodiments, the Cas12j mutant is a nuclease-dead Cas12j protein. Said mutant may be useful e.g. for detecting specific nucleic acid sequences as further detailed herein.

In some embodiments, said altered and/or improved activity is increased specificity for the target nucleic acid sequence.

### Buffers for Optimized Activity of Cas12j Endonucleases

The inventors have a found that the Cas12j endonucleases have one or more altered and/or improved activities, such as improved speed of catalysis or altered nucleic acid cleavage pattern, when the endonuclease is comprised in a medium comprising specific metal ions.

In some embodiments, the endonuclease is comprised in a medium comprising divalent nickel (Ni²⁺), divalent manganese (Mn²⁺) and/or divalent copper (Co²⁺).

In some embodiments, the endonuclease is comprised in a medium comprising divalent nickel (Ni²⁺). In some embodiments, the concentration of Ni²⁺ is at least 0.2 mM, such as at least 0.5 mM, such as at least 1 mM, such as at least 2 mM, such as at least 3 mM, such as at least 4 mM, such as at least 5 mM, such as between 0.2 mM and 5 mM.

In some embodiments, the endonuclease is comprised in a medium comprising divalent manganese (Mn²⁺). In some embodiments, the concentration of Mn²⁺ is least 0.2 mM, such as at least 0.5 mM, such as at least 1 mM, such as at least 2 mM, such as at least 3 mM, such as at least 4 mM, such as at least 5 mM, such as between 0.2 mM and 5 mM.

In some embodiments, the endonuclease is comprised in a medium comprising divalent copper (Co²⁺). In some embodiments, the concentration of Co²⁺ is least 0.2 mM, such as at least 0.5 mM, such as at least 1 mM, such as at least 2 mM, such as at least 3 mM, such as at least 4 mM, such as at least 5 mM, such as between 0.2 mM and 5 mM.

### Polynucleotides and Recombinant Vectors Encoding the Mutant Cas12j Endonuclease

Polynucleotides, nucleic acid sequences and vectors encoding the mutant Cas12j endonucleases as disclosed herein are also provided. The skilled person knows how to design such nucleic acid sequences and/or vectors encoding the desired Cas12j mutant.

In some aspects is provided a polynucleotide encoding the mutant Cas12j endonuclease or orthologue thereof as described herein.

In some embodiments, the mutant Cas12j endonuclease is a mutant CasΦ-1, such as a mutant CasΦ-2, such as a mutant CasΦ-3, such as a mutant CasΦ-4, such as a mutant CasΦ-5, such as a mutant CasΦ-6, such as a mutant CasΦ-7, such as a mutant CasΦ-8, such as a mutant CasΦ-9, or such as a mutant CasΦ-10. In preferred embodiments, the mutant Cas12j endonuclease is a mutant CasΦ-1, such as a mutant CasΦ-2, or such as a mutant CasΦ-3.

In some embodiments, the mutant Cas12j endonuclease is encoded by a polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to a nucleic acid sequence selected from the group consisting of SEQ ID NO: 11, SEQ ID NO: 12 (CasΦ-2), SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 32 and SEQ ID NO: 33.

In some embodiments, the polynucleotide is codon-optimized for expression in a host cell.

In some embodiments, the polynucleotide encodes a mutant CasΦ-1 endonuclease optimized for expression in a bacterial cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 11.

In some embodiments, the polynucleotide encodes a mutant CasΦ-2 endonuclease optimized for expression in a bacterial cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 12.

In some embodiments, the polynucleotide encodes a mutant CasΦ-3 endonuclease optimized for expression in a bacterial cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 13.

In some embodiments, the polynucleotide encodes a C-terminally truncated CasΦ-3 endonuclease optimized for expression in a bacterial cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 32.

In some embodiments, the polynucleotide encodes a mutant CasΦ-4 endonuclease optimized for expression in a bacterial cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 14.

In some embodiments, the polynucleotide encodes a mutant CasΦ-5 endonuclease optimized for expression in a bacterial cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 15.

In some embodiments, the polynucleotide encodes a mutant CasΦ-6 endonuclease optimized for expression in a bacterial cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 16.

In some embodiments, the polynucleotide encodes a mutant CasΦ-7 endonuclease optimized for expression in a bacterial cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 17.

In some embodiments, the polynucleotide encodes a mutant CasΦ-8 endonuclease optimized for expression in a bacterial cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 18.

In some embodiments, the polynucleotide encodes a mutant CasΦ-9 endonuclease optimized for expression in a bacterial cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 19.

In some embodiments, the polynucleotide encodes a mutant CasΦ-10 endonuclease optimized for expression in a bacterial cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 20.

In some embodiments, the polynucleotide encodes a mutant CasΦ-1 endonuclease optimized for expression in a human cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 21.

In some embodiments, the polynucleotide encodes a mutant CasΦ-2 endonuclease optimized for expression in a human cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 22.

In some embodiments, the polynucleotide encodes a mutant CasΦ-3 endonuclease optimized for expression in a human cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 23.

In some embodiments, the polynucleotide encodes a C-terminally truncated CasΦ-3 endonuclease optimized for expression in a human cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 33.

In some embodiments, the polynucleotide encodes a mutant CasΦ-4 endonuclease optimized for expression in a human cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 24.

In some embodiments, the polynucleotide encodes a mutant CasΦ-5 endonuclease optimized for expression in a human cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 25.

In some embodiments, the polynucleotide encodes a mutant CasΦ-6 endonuclease optimized for expression in a human cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 26.

In some embodiments, the polynucleotide encodes a mutant CasΦ-7 endonuclease optimized for expression in a human cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 27.

In some embodiments, the polynucleotide encodes a mutant CasΦ-8 endonuclease optimized for expression in a human cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 28.

In some embodiments, the polynucleotide encodes a mutant CasΦ-9 endonuclease optimized for expression in a human cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 29.

In some embodiments, the polynucleotide encodes a mutant CasΦ-10 endonuclease optimized for expression in a human cell, said polynucleotide comprising or consisting of a nucleic acid sequence with at least 80% sequence identity, such as at least 85%, such as at least 90%, such as at least 95%, such as at least 96%, such as at least 97%, such as at least 98%, such as at least 99% sequence identity to SEQ ID NO: 30.

In some aspects, the present disclosure provides a recombinant vector comprising a polynucleotide or a nucleic acid sequence encoding a mutant Cas12j endonuclease or orthologue thereof as defined above. In some embodiments, said polynucleotide or nucleic acid sequence is operably linked to a promoter.

In some embodiments, the mutant Cas12j endonuclease is a mutant CasΦ-1, such as a mutant CasΦ-2, such as a mutant CasΦ-3, such as a mutant CasΦ-4, such as a mutant CasΦ-5, such as a mutant CasΦ-6, such as a mutant CasΦ-7, such as a mutant CasΦ-8, such as a mutant CasΦ-9, or such as a mutant CasΦ-10. In preferred embodiments, the mutant Cas12j endonuclease is a mutant CasΦ-1, such as a mutant CasΦ-2, or such as a mutant CasΦ-3.

In some embodiments, the recombinant vector further comprises a nucleic acid sequence encoding a guide RNA (crRNA) operably linked to a promoter, wherein the crRNA binds the encoded Cas12j endonuclease and a fragment of nucleic acid with sufficient base pairs to hybridize to a target nucleic acid. The crRNA is further described herein below in the section “Guide RNA (crRNA)”.

### Cells and Systems for Expression of the Mutant Cas12j Endonuclease

Further provided herein are cells and system for expression of the mutant Cas12j endonucleases as disclosed herein.

In some aspects, the present disclosure thus provides a cell capable of expressing the mutant Cas12j endonuclease or orthologue thereof as disclosed herein, the polynucleotide as disclosed herein, or the recombinant vector according as disclosed herein.

In some embodiments, the mutant Cas12j endonuclease is a mutant CasΦ-1, such as a mutant CasΦ-2, such as a mutant CasΦ-3, such as a mutant CasΦ-4, such as a mutant CasΦ-5, such as a mutant CasΦ-6, such as a mutant CasΦ-7, such as a mutant CasΦ-8, such as a mutant CasΦ-9, or such as a mutant CasΦ-10. In preferred embodiments, the mutant Cas12j endonuclease is a mutant CasΦ-1, such as a mutant CasΦ-2, or such as a mutant CasΦ-3.

In some aspects, the present disclosure provides a system for expression of a crRNA-Cas12j complex comprising
a. a polynucleotide as disclosed herein, or a recombinant vector according as disclosed herein comprising a polynucleotide encoding a mutant Cas12j endonuclease or orthologue thereof; and
b. a polynucleotide or a recombinant vector comprising a polynucleotide encoding a guide RNA (crRNA), optionally operably linked to a promoter.

In some embodiments, the mutant Cas12j endonuclease is a mutant CasΦ-1, such as a mutant CasΦ-2, such as a mutant CasΦ-3, such as a mutant CasΦ-4, such as a mutant CasΦ-5, such as a mutant CasΦ-6, such as a mutant CasΦ-7, such as a mutant CasΦ-8, such as a mutant CasΦ-9, or such as a mutant CasΦ-10. In preferred embodiments, the mutant Cas12j endonuclease is a mutant CasΦ-1, such as a mutant CasΦ-2, or such as a mutant CasΦ-3.

In some embodiments, the system further comprises a cell for expression of the polynucleotide or the recombinant vector of a. and b. above.

Suitable host cells for expression of the polynucleotide or the recombinant vector encoding the mutant Cas12j endonuclease as disclosed herein are known to the skilled person. In some embodiments, the cell is a prokaryotic or a eukaryotic cell. In some embodiments, the mutant Cas12j endonuclease is expressed from an Escherichia coli cell. This can be done as is known in the art, for example by introducing a vector comprising the nucleic acid sequence encoding the desired mutant Cas12j endonuclease or orthologue as described herein above in an E. coli cell, such as by electroporation or chemical transformation. The protein may be isolated and/or purified as is known in the art.

Guide RNA (crRNA)

In order to function as an endonuclease, the crRNA-Cas12j complex requires not only the Cas12j effector protein, but also a guide RNA (crRNA), which is responsible for recognition of the target nucleic acid to be cleaved.

The crRNA comprises or consists of a constant region and of a variable region. The constant region consists of 23-25 nucleotides and is constant for all complexes derived from a given organism. For optimal activity of the crRNA-Cas12j complex, it may be important to design the crRNA based on the constant region specific for the Cas12j nuclease or its orthologue that is used.

In some embodiments, the constant region is specific for CasΦ-1 and has the sequence as defined in SEQ ID NO: 34. In some embodiments, the constant region is specific for CasΦ-2 and has the sequence as defined in SEQ ID NO: 35. In some embodiments, the constant region is specific for CasΦ-3 and has the sequence as defined in SEQ ID NO: 36.

The variable region consists of between 9 and 20 nucleotides, such as 9, 10, 11, 12, 13, 14, 15, 16, 17, 18, 19, or 20 nucleotides. The variable region is the region of the crRNA which is thought to be responsible for target recognition. Modifying the sequence of the variable region can thus be taken advantage of in order for the crRNA-Cas12j complex to be able to specifically cleave different target nucleic acids. In contrast to the constant region, the variable region is not specific to the specific Cas12j endonuclease.

Accordingly, in some embodiments, the crRNA consists of a constant region of 23 nucleotides and a variable region of 9 nucleotides, and the crRNA has a total length of 32 nucleotides. In some embodiments, the crRNA consists of a constant region of 23 nucleotides and a variable region of 10 nucleotides, and the crRNA has a total length of 33 nucleotides. In some embodiments, the crRNA consists of a constant region of 23 nucleotides and a variable region of 11 nucleotides, and the crRNA has a total length of 34 nucleotides. In some embodiments, the crRNA consists of a constant region of 23 nucleotides and a variable region of 12 nucleotides, and the crRNA has a total length of 35 nucleotides. In some embodiments, the crRNA consists of a constant region of 23 nucleotides and a variable region of 13 nucleotides, and the crRNA has a total length of 36 nucleotides. In some embodiments, the crRNA consists of a constant region of 23 nucleotides and a variable region of 14 nucleotides, and the crRNA has a total length of 37 nucleotides. In some embodiments, the crRNA consists of a constant region of 23 nucleotides and a variable region of 15 nucleotides, and the crRNA has a total length of 38 nucleotides. In some embodiments, the crRNA consists of a constant region of 23 nucleotides and a variable region of 16 nucleotides, and the crRNA has a total length of 39 nucleotides. In some embodiments, the crRNA consists of a constant region of 23 nucleotides and a variable region of 17 nucleotides, and the crRNA has a total length of 40 nucleotides. In some embodiments, the crRNA consists of a constant region of 23 nucleotides and a variable region of 18 nucleotides, and the crRNA has a total length of 41 nucleotides. In some embodiments, the crRNA consists of a constant region of 23 nucleotides and a variable region of 19 nucleotides, and the crRNA has a total length of 42 nucleotides. In some embodiments, the crRNA consists of a constant region of 23 nucleotides and a variable region of 20 nucleotides, and the crRNA has a total length of 43 nucleotides.

In some embodiments, the crRNA consists of a constant region of 24 nucleotides and a variable region of 9 nucleotides, and the crRNA has a total length of 33 nucleotides. In some embodiments, the crRNA consists of a constant region of 24 nucleotides and a variable region of 10 nucleotides, and the crRNA has a total length of 34 nucleotides. In some embodiments, the crRNA consists of a constant region of 24 nucleotides and a variable region of 11 nucleotides, and the crRNA has a total length of 35 nucleotides. In some embodiments, the crRNA consists of a constant region of 24 nucleotides and a variable region of 12 nucleotides, and the crRNA has a total length of 36 nucleotides. In some embodiments, the crRNA consists of a constant region of 24 nucleotides and a variable region of 13 nucleotides, and the crRNA has a total length of 37 nucleotides. In some embodiments, the crRNA consists of a constant region of 24 nucleotides and a variable region of 14 nucleotides, and the crRNA has a total length of 38 nucleotides. In some embodiments, the crRNA consists of a constant region of 24 nucleotides and a variable region of 15 nucleotides, and the crRNA has a total length of 39 nucleotides. In some embodiments, the crRNA consists of a constant region of 24 nucleotides and a variable region of 16 nucleotides, and the crRNA has a total length of 40 nucleotides. In some embodiments, the crRNA consists of a constant region of 24 nucleotides and a variable region of 17 nucleotides, and the crRNA has a total length of 41 nucleotides. In some embodiments, the crRNA consists of a constant region of 24 nucleotides and a variable region of 18 nucleotides, and the crRNA has a total length of 42 nucleotides. In some embodiments, the crRNA consists of a constant region of 24 nucleotides and a variable region of 19 nucleotides, and the crRNA has a total length of 43 nucleotides. In some embodiments, the crRNA consists of a constant region of 24 nucleotides and a variable region of 20 nucleotides, and the crRNA has a total length of 44 nucleotides.

In some embodiments, the crRNA consists of a constant region of 25 nucleotides and a variable region of 9 nucleotides, and the crRNA has a total length of 34 nucleotides. In some embodiments, the crRNA consists of a constant region of 25 nucleotides and a variable region of 10 nucleotides, and the crRNA has a total length of 35 nucleotides. In some embodiments, the crRNA consists of a constant region of 25 nucleotides and a variable region of 11 nucleotides, and the crRNA has a total length of 36 nucleotides. In some embodiments, the crRNA consists of a constant region of 25 nucleotides and a variable region of 12 nucleotides, and the crRNA has a total length of 37 nucleotides. In some embodiments, the crRNA consists of a constant region of 25 nucleotides and a variable region of 13 nucleotides, and the crRNA has a total length of 38 nucleotides. In some embodiments, the crRNA consists of a constant region of 25 nucleotides and a variable region of 14 nucleotides, and the crRNA has a total length of 39 nucleotides. In some embodiments, the crRNA consists of a constant region of 25 nucleotides and a variable region of 15 nucleotides, and the crRNA has a total length of 40 nucleotides. In some embodiments, the crRNA consists of a constant region of 25 nucleotides and a variable region of 16 nucleotides, and the crRNA has a total length of 41 nucleotides. In some embodiments, the crRNA consists of a constant region of 25 nucleotides and a variable region of 17 nucleotides, and the crRNA has a total length of 42 nucleotides. In some embodiments, the crRNA consists of a constant region of 25 nucleotides and a variable region of 18 nucleotides, and the crRNA has a total length of 43 nucleotides. In some embodiments, the crRNA consists of a constant region of 25 nucleotides and a variable region of 19 nucleotides, and the crRNA has a total length of 44 nucleotides. In some embodiments, the crRNA consists of a constant region of 25 nucleotides and a variable region of 20 nucleotides, and the crRNA has a total length of 45 nucleotides.

The skilled person will have no difficulty in designing a variable region capable of binding the desired target nucleic acid. The variable region has a sequence which is the reverse complement of the target nucleic acid.

The crRNA thus consists of a constant region of 23, 24 or 25 nucleotides, and of a variable region consisting of between 9 and 20 nucleotides, such that said crRNA is at least 32 nucleotides in length, 33 nucleotides in length, 34 nucleotides in length, 35 nucleotides in length, 36 nucleotides in length, 37 nucleotides in length, 38 nucleotides in length, 39 nucleotides in length, 40 nucleotides in length, 41 nucleotides in length, 42 nucleotides in length, 43 nucleotides in length, 44 nucleotides in length or 45 nucleotides in length.

Recognition and binding of the crRNA-Cas12j complex to a target nucleic acid relies on the crRNA binding to the target nucleic acid. This is dependent on the presence of a PAM (protospacer adjacent motif) sequence in the target nucleic acid. In some embodiments, the crRNA is designed to bind to a target nucleic acid sequence comprising a PAM sequence at the 5′-end. In some embodiments, the PAM sequence comprises or consists of the sequence 5′-TTN-3′. The crRNA preferably does not hybridize to the PAM itself.

Once a guide RNA sequence has been designed, the guide RNA can be synthesised by known methods. For example, DNA oligonucleotides corresponding to the reverse complemented sequence of the target site may be ordered from a company selling oligonucleotides. These oligonucleotides may contain a 24 base long T7 priming sequence. These DNA duplexes may then be used as template in a transcription reaction carried with T7 RNA polymerase. For example, the reaction may consist of incubation at 37° C. for at least 1 hour. The reaction may be stopped using 2× stop solution, for example 50 mM EDTA, 20 mM Tris-HCl PH 8.0 and 8 M Urea. The RNA may be purified by methods known in the art, such as LiCl precipitation.

Use of a crRNA-Cas12j Endonuclease Complex for Genome Editing

The mutant Cas12j endonucleases of the present disclosure may advantageously be used for genome editing.

In some aspects, the present disclosure provides a method of introducing a nucleic acid break in a first target nucleic acid, comprising the steps of:
a. designing a guide-RNA (crRNA) capable of recognising a second target nucleic acid comprising a protospacer adjacent motif (PAM);
b. contacting the crRNA of step a. with a mutant Cas12j endonuclease or orthologue thereof, wherein the mutant Cas12j endonuclease or orthologue thereof is as disclosed herein, or encoded by a polynucleotide or a vector as disclosed herein, thereby obtaining a crRNA-Cas12j complex capable of binding to said second target nucleic acid, and
c. contacting the crRNA and the mutant Cas12j endonuclease with said first target nucleic acid,

thereby introducing one or more nucleic acid breaks in the first target nucleic acid.

In some embodiments, the mutant Cas12j endonuclease is a mutant CasΦ-1, such as a mutant CasΦ-2, such as a mutant CasΦ-3, such as a mutant CasΦ-4, such as a mutant Caso-5, such as a mutant CasΦ-6, such as a mutant CasΦ-7, such as a mutant CasΦ-8, such as a mutant CasΦ-9, or such as a mutant CasΦ-10. In preferred embodiments, the mutant Cas12j endonuclease is a mutant CasΦ-1, such as a mutant CasΦ-2, or such as a mutant CasΦ-3.

In some embodiments, steps b. and c. of the method disclosed herein above occur simultaneously. In some embodiments, steps b. and c. of the method disclosed herein above occur one after the other.

In some aspects, the present disclosure provides the use of a crRNA-Cas12j complex in a method for introducing a nucleic acid break in a first target nucleic acid, wherein:
a. a mutant Cas12j endonuclease or orthologue thereof is contacted with a guide RNA (crRNA), thereby obtaining a crRNA-Cas12j complex capable of recognizing a second target nucleic acid, the second target nucleic acid comprising a protospacer adjacent motif (PAM), and wherein the Cas12j endonuclease or orthologue thereof is according to any one of claim 1 to Error! Reference source not found.;
b. the crRNA-Cas12j complex is contacted with the first target nucleic acid;

whereby a nucleic acid break is made in the first target nucleic acid sequence.

In some embodiments, the mutant Cas12j endonuclease is a mutant CasΦ-1, such as a mutant CasΦ-2, such as a mutant CasΦ-3, such as a mutant CasΦ-4, such as a mutant CasΦ-5, such as a mutant CasΦ-6, such as a mutant CasΦ-7, such as a mutant CasΦ-8, such as a mutant CasΦ-9, or such as a mutant CasΦ-10. In preferred embodiments, the mutant Cas12j endonuclease is a mutant CasΦ-1, such as a mutant CasΦ-2, or such as a mutant CasΦ-3.

In some embodiments, the first target nucleic acid and the second target nucleic acid are DNA. In some embodiments, the first target nucleic acid and the second target nucleic acid are RNA. In some embodiments, the first target nucleic acid is DNA and the second target nucleic acid is RNA. In some embodiments, the first target nucleic acid is RNA and the second target nucleic acid is DNA.

In some embodiments, the first and/or second target nucleic acid is double stranded DNA. In some embodiments, the first and second target nucleic acids are a complement of each other. In some embodiments, the first and second target nucleic acids are the same stretch of a double-stranded nucleic acid.

In some embodiments, the nucleic acid break is a single-stranded break. In some embodiments, the single-stranded nucleic acid break is in the first target sequence. In some embodiments, the single-stranded nucleic acid break is in the second target sequence. In some embodiments, the single-stranded nucleic acid break is made in a specific recognition nucleotide sequence of the first target nucleic acid.

In some embodiments, the nucleic acid break is a double-stranded break. In this case, a nucleic acid break is made in both the first and the second target sequences. In some embodiments, the double-stranded break is a staggered double-stranded break. In some embodiments, the double-stranded break is a blunt double-stranded break.

In some embodiments, the mutant Cas12j endonuclease or orthologue thereof is encoded by a polynucleotide or a vector as disclosed herein. In some embodiments, the mutant Cas12j endonuclease or orthologue thereof is as disclosed herein. In some embodiments, the mutant Cas12j endonuclease or orthologue thereof is as disclosed herein and is encoded by a polynucleotide or a vector as disclosed herein.

In some embodiments, the second target nucleic acid comprises or consists of a recognition sequence comprising a sequence of at least 15 consecutive nucleotides, such as at least 16 consecutive nucleotides, such as at least 17 consecutive nucleotides, such as at least 18 consecutive nucleotides, such as at least 19 consecutive nucleotides, such as at least 20 consecutive nucleotides, such as at least 21 consecutive nucleotides, such as at least 22 consecutive nucleotides, such as at least 23 consecutive nucleotides, such as at least 24 consecutive nucleotides, such as at least 25 consecutive nucleotides, such as at least 26 consecutive nucleotides, such as at least 27 consecutive nucleotides, with the proviso that the 3 nucleic acids at the 5′-end consist of a PAM sequence.

In some embodiments, the first target nucleic acid is genomic DNA. In some embodiments, the first target nucleic acid is chromatin. In some embodiments, the first target nucleic acid is a nucleosome. In some embodiments, the first target nucleic acid is plasmid DNA. In some embodiments, the first target nucleic acid is methylated DNA. In some embodiments, the first target nucleic acid is synthetic DNA. In some embodiments, the first target nucleic acid is a DNA fragment. In some embodiments, the second target nucleic acid is genomic DNA. In some embodiments, the second target nucleic acid is chromatin. In some embodiments, the second target nucleic acid is a nucleosome. In some embodiments, the second target nucleic acid is plasmid DNA. In some embodiments, the second target nucleic acid is methylated DNA. In some embodiments, the second target nucleic acid is synthetic DNA. In some embodiments, the second target nucleic acid is a DNA fragment.

In some embodiments, the method as disclosed herein is performed ex vivo. In some embodiments, the method as disclosed herein is performed in a cell in vitro.

As mentioned herein above, the first and the second target nucleic acid may be the same stretch of double-stranded nucleic acid. In this case, a double-stranded break may be introduced in both the first and the second target nucleic acids

Thus, in some aspects is provided an in vitro method of introducing a site-specific, double-stranded break at a second target nucleic acid in a mammalian cell, the method comprising introducing into the mammalian cell a crRNA-Cas12j complex, wherein the Cas12j is a mutant Cas12j endonuclease or orthologue as disclosed herein, and wherein the crRNA is specific for the second target nucleic acid.

In some embodiments, the mutant Cas12j endonuclease is a mutant CasΦ-1, such as a mutant CasΦ-2, such as a mutant CasΦ-3, such as a mutant CasΦ-4, such as a mutant CasΦ-5, such as a mutant CasΦ-6, such as a mutant CasΦ-7, such as a mutant CasΦ-8, such as a mutant CasΦ-9, or such as a mutant CasΦ-10. In preferred embodiments, the mutant Cas12j endonuclease is a mutant CasΦ-1, such as a mutant CasΦ-2, or such as a mutant CasΦ-3.

Use of a crRNA-Cas12j Endonuclease Complex for Detection and/or Quantification of a Target DNA Sequence

Some of the mutant Cas12j endonucleases of the present disclosure are capable of introducing single strand breaks only in a first target sequence, which is not hybridized by the crRNA of the crRNA-Cas12j complex. Thus, in some embodiments when the crRNA of a crRNA-Cas12j complex recognizes and hybridizes to a second target sequence, the nickase activity of the mutant Cas12j of said complex will be activated and it will introduce one or more single strand break at sites of the first target sequence. Moreover, the second target nucleic acid will not be cleaved by the Cas12j endonuclease, which will therefore stay in an active state for a longer period of time and possibly cleave more than one first target sequences. Provided that the first target sequence is labelled in a way that a signal will be released upon cleavage of said first target sequence, the described method will thus allow detection of the second target sequence.

These mutant Cas12j endonucleases, when in a crRNA-Cas12j complex, can thus be used to detect and quantify a second target sequence, with the help of a provided labelled first target sequence.

In some embodiments, the second target nucleic acid is a target nucleic acid of interest.

In some aspects is therefore provided a method for detection of a second target nucleic acid in a sample, the method comprising:
a. Providing a crRNA-Cas12j complex, wherein the Cas12j is a mutant Cas12j endonuclease or orthologue thereof as disclosed herein, and wherein the crRNA is specific for the second target nucleic acid;
b. Providing a labelled ssDNA, wherein the ssDNA is labelled with at least one set of interactive labels comprising at least one dye and at least one quencher;
c. Contacting the crRNA-Cas12j complex and the ssDNA with the sample, wherein the sample comprises at least one second target nucleic acid; and
d. Detecting cleavage of the ssDNA by detecting a fluorescent signal from the fluorophore,

thereby detecting the presence of the second target nucleic acid in the sample, wherein step c. optionally comprises activation of the crRNA-Cas12j complex.

In some embodiments, the mutant Cas12j endonuclease is a mutant CasΦ-1, such as a mutant CasΦ-2, such as a mutant CasΦ-3, such as a mutant CasΦ-4, such as a mutant CasΦ-5, such as a mutant CasΦ-6, such as a mutant CasΦ-7, such as a mutant CasΦ-8, such as a mutant CasΦ-9, or such as a mutant CasΦ-10. In preferred embodiments, the mutant Cas12j endonuclease is a mutant CasΦ-1, such as a mutant CasΦ-2, or such as a mutant CasΦ-3.

In step c. the crRNA-Cas12j complex and the ssDNA are contacted with at least one second target nucleic acid, and the recognition and binding of the crRNA with the second target nucleic acid, such as single-stranded or double-stranded target DNA, results in activation of the crRNA-Cas12j complex, which is then capable of introducing single strand breaks, such as cleaving, the ssDNA.

Hence, step c. may comprise activation of the crRNA-Cas12j complex.

The method may further comprise the step of determining the level and/or concentration of the second target nucleic acid, wherein the level and/or concentration of the second target nucleic acid is correlated to the cleaved ssDNA.

As explained above, in some embodiments the mutant Cas12j endonuclease disclosed herein will not cleave the second target nucleic acid and thus will stay active for a period of time which may be sufficient for cleaving multiple times in the first target nucleic acid sequence, which in the method described herein may be the labelled ssDNA or a fragment thereof. The more first target nucleic acid molecules are cleaved by the crRNA-Cas12j complex after hybridization of the crRNA-Cas12j complex to a second target nucleic acid, the higher the signal and thus the higher the sensitivity of the method. This is an advantage of the disclosed mutant Cas12j over other Cas12j endonucleases.

Hence, the method disclosed herein has high sensitivity and may allow detection of the second target nucleic acid at concentrations in the nanomolar range and below, such as at concentrations in the picomolar range and below, such as at concentrations in the femtomolar range or below. For example, the method disclosed herein allows detection of a second target nucleic acid at concentrations in the attomolar range or below.

In some embodiments, the mutant Cas12j endonuclease disclosed herein will cleave the second target nucleic acid and thus will stay active only until the cleaved second target nucleic acid is released.

The ssDNA may be labelled in at least one base in any position along the chain. For example, the ssDNA is labelled in one base in any position along the chain, such as in at least two bases in any position along the chain, such as in at least three bases in any position along the chain, such as in at least four bases in any position along the chain.

The ssDNA may be labelled with at least one set of interactive labels comprising at least one dye and at least one quencher.

In some embodiments, the at least one dye is a fluorophore.

Thus, the cleavage of the ssDNA in step d. of the method comprises detecting a fluorescent signal resulting from cleavage of the ssDNA.

In some embodiments, the at least one fluorophore is selected from the group comprising black hole quencher (BHQ) 1, BHQ2, and BHQ3, Cosmic Quencher (e.g. from Biosearch Technologies, Novato, USA), Excellent Bioneer Quencher (EBQ) (e.g. from Bioneer, Daejeon, Korea) or a combination hereof.

In some embodiments, the at least one quencher is selected from the group comprising black hole quencher (BHQ) 1, BHQ2, and BHQ3 (from Biosearch Technologies, Novato, USA).

A fluorophore which may be useful in the present invention may include any fluorescent molecule known in the art. Examples of fluorophores are: Cy2™ Cfflfi), YO-PRn™-1 (509), YDYO™-1 (509), Calrein (517), FITC (518), FluorX™ (519), Alexa™ (520), Rhodamine 110 (520), Oregon Green™ 500 (522), Oregon Green™ 488 (524), RiboGreen™ (525), Rhodamine Green™ (527), Rhodamine 123 (529), Magnesium Green™ (531), Calcium Green™ (533), TO-PRO™-I (533), TOTOI (533), JOE (548), 30 BODIPY530/550 (550), Dii (565), BODIPY TMR (568), BODIPY558/568 (568), BODIPY564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAMRA (582), Rhodamine Red™ (590), Cy3.5(TM) (596), ROX (608), Calcium Crimson™ (615), Alexa™ 594 35 (615), Texas Red (615), Nile Red (628), YO-PRO™-3 (631), YOYO™-3 (631), RP3649PC00 phycocyanin (642), C-Phycocyanin (648), TO-PRO™-3 (660), TOT03 (660), DiD DiIC (5) (665), Cy5™ (670), Thiadicarbocyanine (671), Cy5.5 (694), HEX (556), TET (536), Biosearch Blue (447), CAL Fluor Gold 540 (544), CAL Fluor Orange 560 (559), CAL Fluor Red 590 (591), CAL Fluor Red 610 (610), CAL Fluor Red 635 (637), FAM (520), 6-Carboxyfluorescein (6-FAM), Fluorescein (520), Fluorescein-C3 (520), Pulsar 650 (566), Quasar 570 (667), Quasar 670 (705) and Quasar 705 (610). The number in parenthesis is a maximum emission wavelength in nanometers.

A non-fluorescent black quencher molecule capable of quenching a fluorescence of a wide range of wavelengths or a specific wavelength may be used in the present invention.

Suitable pairs of fluorophores/quenchers are known in the art.

As disclosed herein, the mutant Cas12j endonuclease may additionally comprise a protein tag, such as fluorescent protein or affinity tag. In some embodiments, the endonuclease activity of the mutant Cas12j has been abrogated and no nucleic acid breaks will thus be introduced in either the first or the second target nucleic acid sequences. These mutants are especially useful for detection and/or quantification of a target nucleic acid sequence.

Thus, in some aspects is also provided a method for detection and optionally quantification of a second target nucleic acid in a sample, the method comprising:
a. Providing a crRNA-Cas12j complex, wherein the Cas12j is a mutant Cas12j endonuclease or orthologue thereof as disclosed herein, wherein
   i. the mutant Cas12j has an abrogated endonuclease activity;
   ii. the mutant Cas12j comprises a detectable protein label; and
   iii, the crRNA is specific for the second target nucleic acid;
b. Contacting the crRNA-Cas12j complex with the sample, wherein the sample comprises at least one second target nucleic acid; and
c. Detecting and optionally quantifying the presence of the second target nucleic acid by detecting the protein label, such as a fluorescent signal.

In some embodiments, the mutant Cas12j endonuclease is a mutant CasΦ-1, such as a mutant CasΦ-2, such as a mutant CasΦ-3, such as a mutant CasΦ-4, such as a mutant CasΦ-5, such as a mutant CasΦ-6, such as a mutant CasΦ-7, such as a mutant Caso-8, such as a mutant CasΦ-9, or such as a mutant CasΦ-10. In preferred embodiments, the mutant Cas12j endonuclease is a mutant CasΦ-1, such as a mutant CasΦ-2, or such as a mutant CasΦ-3.

The methods as disclosed herein may be used to detect presence and levels of any nucleic acid and thus the sample may be any sample comprising nucleic acid and appropriately treated, for example to eliminate proteases. The sample may comprise DNA and/or RNA. The sample may be a sample suspected of comprising the second target nucleic acid. The sample may be culture extract of any prokaryotic or eukaryotic cell culture, body fluid of a mammal, such as of a human.

The second target nucleic acid may be a nucleic acid fragment of a viral genome, a microbial genome, a gene, such as an oncogene, or of a genome of a pathogen.

In some embodiments, the second target nucleic acid is a nucleic acid sequence associated with a human disease. This may be a biomarker for a human disease, e.g. such as a specific mutation or single-nucleotide polymorphism often associated with a specific disease.

The second target nucleic acid may also be a mutated nucleic acid sequence, for example a single nucleotide polymorphism (SNP).

The mutant Cas12j endonuclease used in the methods for detection of a second target nucleic acid in a sample may be any of the mutants described herein.

Use of a crRNA-Cas12j Endonuclease Complex for Diagnosis of a Disease

The present disclosure also relates to methods for diagnosis of any disease which is associated with increased/reduced gene expression and/or with the presence of exogenous genetic material.

In some aspects is provided an in vitro method for diagnosis of a disease in a subject, the method comprising:
a. Providing a crRNA-Cas12j complex, wherein the Cas12j is a mutant Cas12j endonuclease or orthologue thereof as disclosed herein, and wherein the crRNA is specific for a second target nucleic acid;
b. Providing a labelled ssDNA, wherein the ssDNA is labelled with at least one set of interactive labels comprising at least one dye and at least one quencher;
c. Providing a sample from the subject, wherein said sample comprises or is suspected of comprising the second target nucleic acid; and
d. Determining the level and/or concentration of the second target nucleic acid as defined in any one of the preceding claims,

wherein the second target nucleic acid is a nucleic acid fragment that correlates with the disease, such as wherein the second target nucleic acid is a biomarker of the disease,

thereby diagnosing a disease in a subject.

In some embodiments, the mutant Cas12j endonuclease is a mutant CasΦ-1, such as a mutant CasΦ-2, such as a mutant CasΦ-3, such as a mutant CasΦ-4, such as a mutant CasΦ-5, such as a mutant CasΦ-6, such as a mutant CasΦ-7, such as a mutant Caso-8, such as a mutant CasΦ-9, or such as a mutant CasΦ-10. In preferred embodiments, the mutant Cas12j endonuclease is a mutant CasΦ-1, such as a mutant CasΦ-2, or such as a mutant CasΦ-3.

The method for diagnosis of a disease in a subject may further comprise a step of treating said disease. For example, the method may further comprise treating said disease by administering a therapeutically effective agent.

In some embodiments, the disease is an infectious disease.

In some aspects is thus provided an in vitro method for diagnosis of an infectious disease in a subject, the method comprising:
a. Providing a crRNA-Cas12j complex, wherein the Cas12j is a mutant Cas12j endonuclease or orthologue thereof as disclosed herein, and wherein the crRNA is specific for a second target nucleic acid;
b. Providing a labelled ssDNA, wherein the ssDNA is labelled with at least one set of interactive labels comprising at least one dye and at least one quencher;
c. Providing a sample from the subject, wherein said sample comprises or is suspected of comprising the second target nucleic acid; and
d. Determining the level and/or concentration of the second target nucleic acid as defined in any one of the preceding claims,

wherein the second target nucleic acid is a nucleic acid of the genome of an infectious agent causing the disease or a fragment thereof,

thereby diagnosing an infectious disease in a subject.

In some embodiments, the mutant Cas12j endonuclease is a mutant CasΦ-1, such as a mutant CasΦ-2, such as a mutant CasΦ-3, such as a mutant CasΦ-4, such as a mutant CasΦ-5, such as a mutant CasΦ-6, such as a mutant CasΦ-7, such as a mutant CasΦ-8, such as a mutant CasΦ-9, or such as a mutant CasΦ-10. In preferred embodiments, the mutant Cas12j endonuclease is a mutant CasΦ-1, such as a mutant CasΦ-2, or such as a mutant CasΦ-3.

The interactive label may for example comprise a luminescent label.

In some embodiments, the method further comprises a step of treating said infectious disease. In some embodiments, the method further comprises treating said infectious disease by administration of a therapeutically effective compound.

The method for diagnosis of an infectious disease in a subject may further comprise the step of comparing the level and/or concentration of said second target nucleic acid with a cut-off value,
wherein said cut-off value is determined from the concentration range of said second target nucleic acid in healthy subjects, such as subjects who do not present with the infectious disease,
wherein a level and/or concentration that is greater than the cut-off value indicates the presence of the infectious disease.

An infectious disease is any disease caused by an infectious agent such as viruses, viroids, prions, bacteria, nematodes, parasitic roundworms, pinworms, arthropods, fungi, ringworm and macroparasites.

Thus, the second target nucleic acid may be a genome or fragment thereof of an infectious agent selected from the group consisting of viruses, viroids, prions, bacteria, nematodes, parasitic roundworms, pinworms, arthropods, fungi, ringworm and macroparasites.

The method disclosed herein may be used to diagnose an infection disease in a human.

Thus, the sample comprising the second target nucleic acid may by a sample taken from a human body. For example, the sample may be a human body fluid selected from the group consisting of blood, whole blood, plasma, serum, urine, saliva, tears, cerebrospinal fluid and semen.

The mutant Cas12j endonuclease used in the methods for diagnosis of a disease may be any of the mutants described herein.

### EXAMPLES

### Example 1—Structure of the Mini-RNA-Guided Endonuclease CRISPR-CasΦ3

### Materials and Methods

### Plasmid Preparation, Protein Expression and Purification

CasΦ3 cDNA was synthetized and cloned with a C-terminal hexahistidine (His)-tag into pET-21 vector (Genewiz). CasΦ3 mutants were generated with the In-Fusion cloning kit (Takara). To generate CasΦ3-ACT, a TEV cleavage site (ENLYFQG) was generated after the residue M726. His-tagged CasΦ3 was expressed from pET-21 in E. coli BL21 pRARE cells. E. coli cultures were grown at 37° C. in liquid Terrific Broth (TB) medium with 34 mg/l chloramphenicol and 100 mg/l ampicillin to an optical density at 600 nm of ~ 0.8. Overexpression of proteins was induced with 150 nM of IPTG for 16 h at 16° C. Cells were harvested by centrifugation and resuspended in lysis buffer (50 mM HEPES pH7.5, 2M NaCl, 5 mM MgCl₂, 1 tablet of Complete Inhibitor cocktail EDTA Free (Roche) per 50 ml, 50 U/ml Benzonase, 1 mg/ml lysozyme). Lysis was completed by one freeze-thaw cycle and sonication. Cell extract was diluted to a final salt concentration of 500 mM, and high-speed centrifuged (10,000×g, 45 min) to separate the soluble fraction from the insoluble fraction and the cell debris. The soluble fraction was loaded into a 5 ml HisTrap FF Crude column (Cytiva) equilibrated in buffer IMAC-A (20 mM HEPES pH7.5, 500 mM NaCl, 20 mM Imidazole), and bound proteins were eluted by stepwise increase of the imidazole concentration with buffer IMAC-B (20 mM HEPES pH7.5, 200 mM KCl, 500 mM Imidazole). CasΦ3 proteins eluted at ~150 mM Imidazole. In the case of CasΦ3-ACT, the C-terminal segment (residues 727-766) was cleaved by incubating the protein with 0.3 mg TEV protease in TEV buffer (20 mM HEPES pH 7.5, 150 mM NaCl, 1 mM EDTA, 0.5 mM TCEP) for 16 h at 4° C. Fractions containing CasΦ3 were pooled, concentrated and further purified by size exclusion chromatography (SEC) using a HiLoad 16/600 Superdex 200 column (Cytiva) equilibrated in SEC buffer (20 mM HEPES pH7.5, 500 mM KCl, 0.5 mM TCEP). Fractions containing pure protein were pooled, concentrated to 5-10 g/L, flash-frozen in liquid nitrogen and stored at −80° C.

### Cleavage Assays

Fluorescein (FAM)-labeled DNA oligonucleotide at 5′ or 3′ ends, unlabeled DNA and RNA oligonucleotides were purchased from Integrated DNA technologies (IDT). dsDNA substrates were prepared by mixing ssDNA oligos to a final concentration of 80 μM in annealing buffer (20 mM HEPES pH7.5, 200 mM KCl), denaturation at 95° C. for 10 min and gradually temperature decrease to 4° C. during 20 minutes in a thermal cycler (Applied Biosystems). Ribonucleoprotein complexes (RNP) of CasΦ3 were formed by mixing an equal volume of 50 UM CasΦ3 and 50 μM CasΦ3 mature crRNA (IDT).

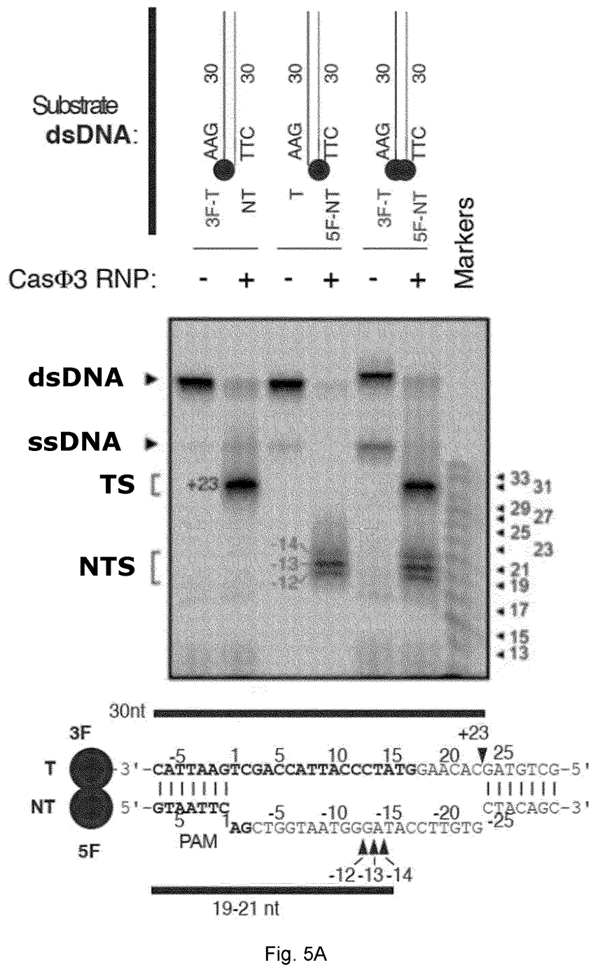

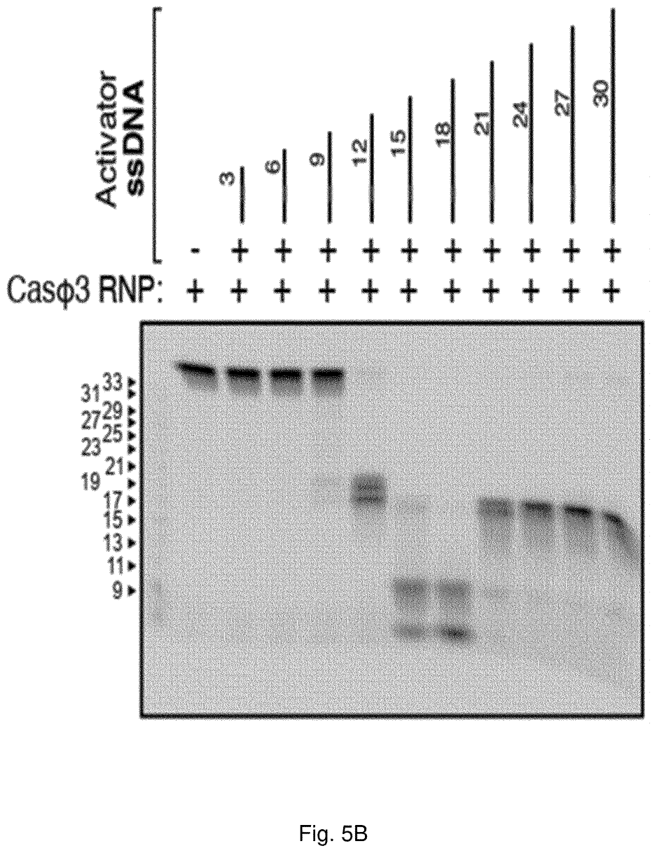

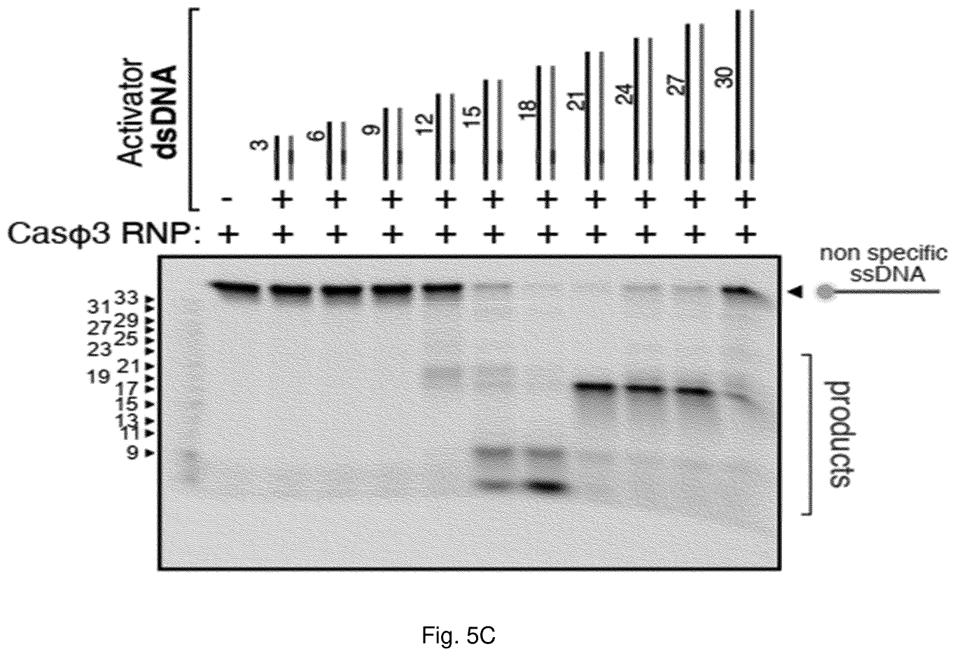

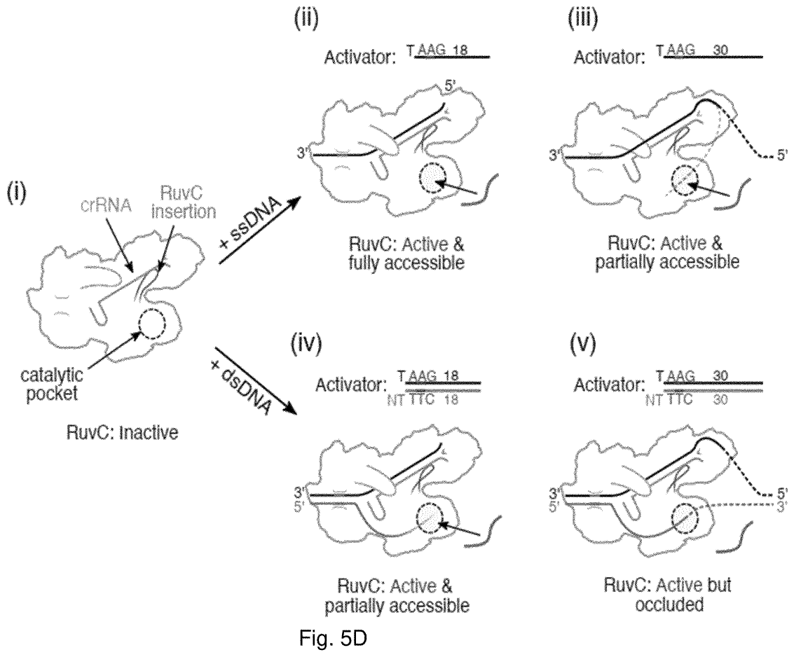

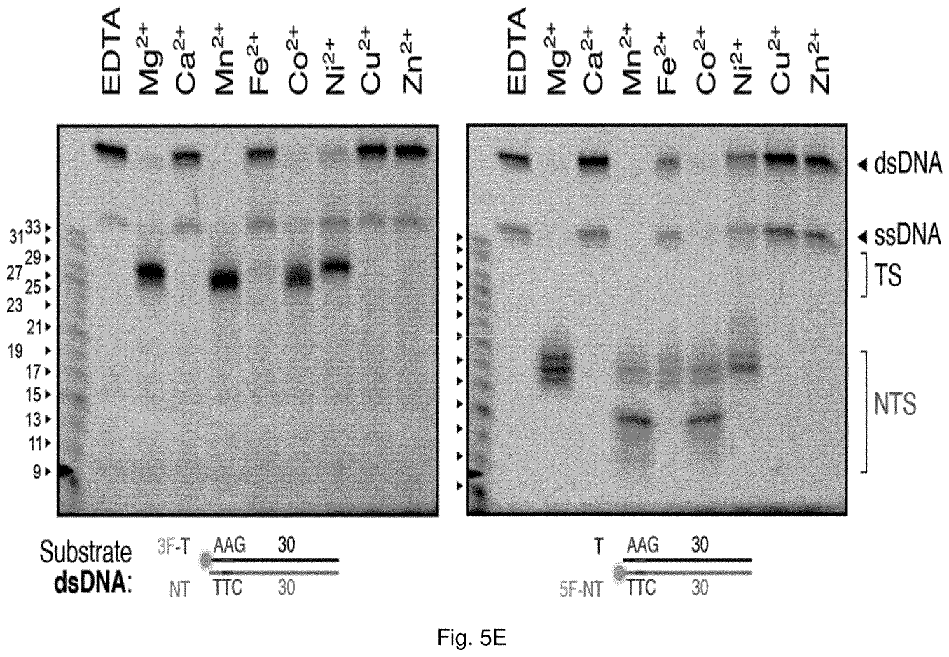

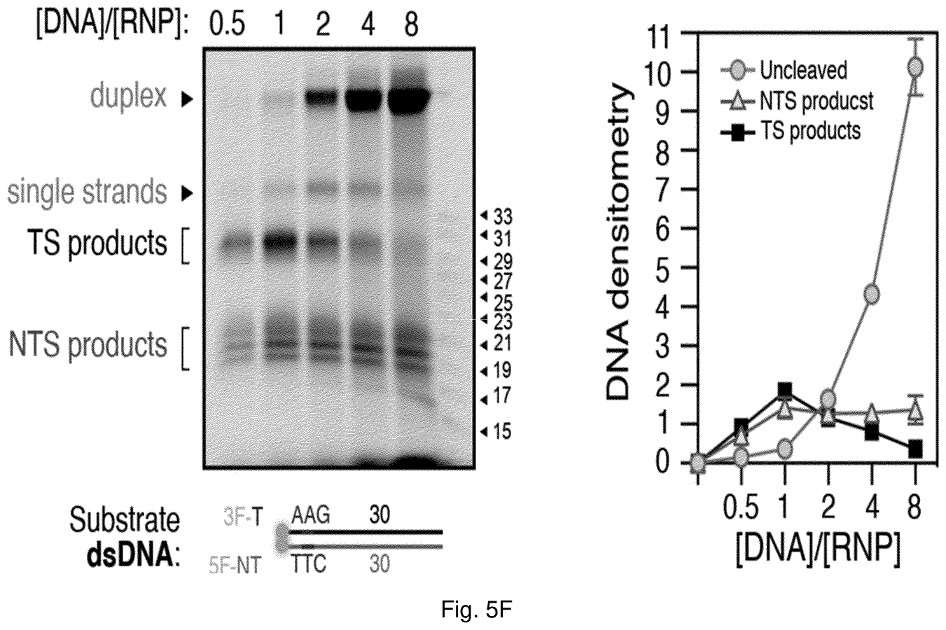

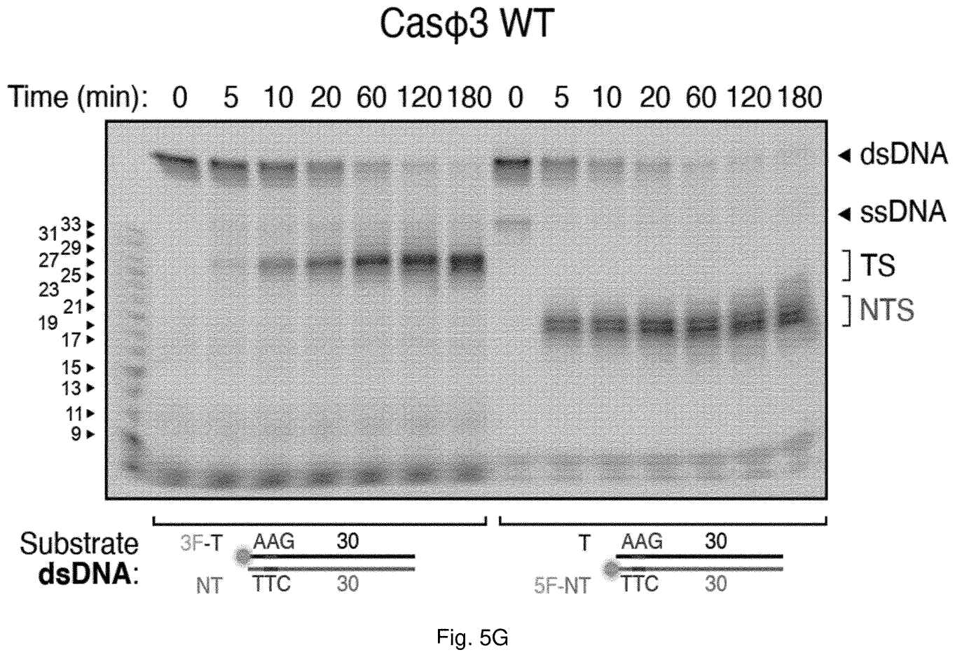

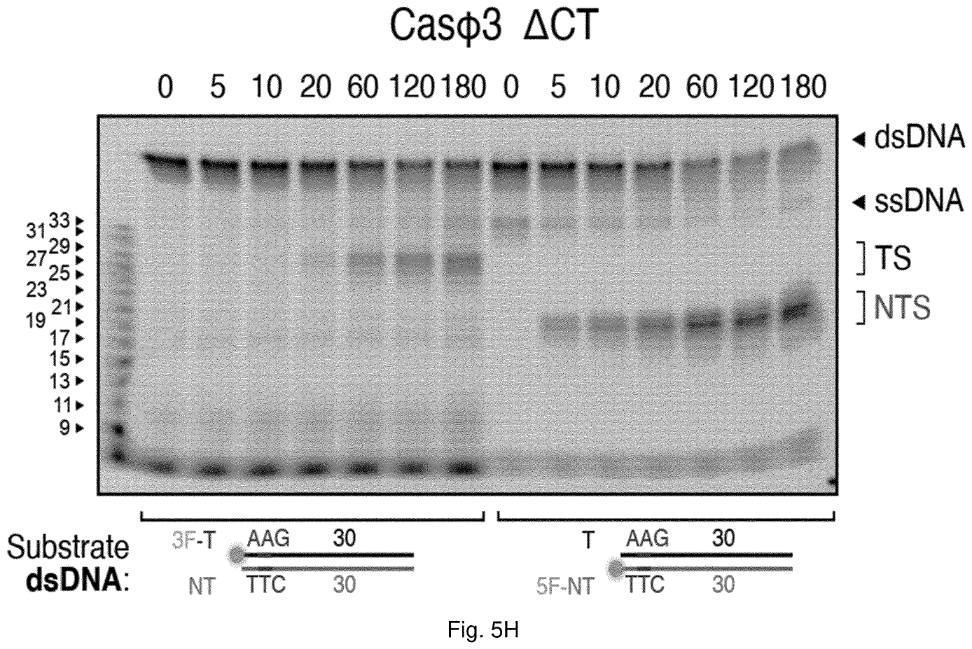

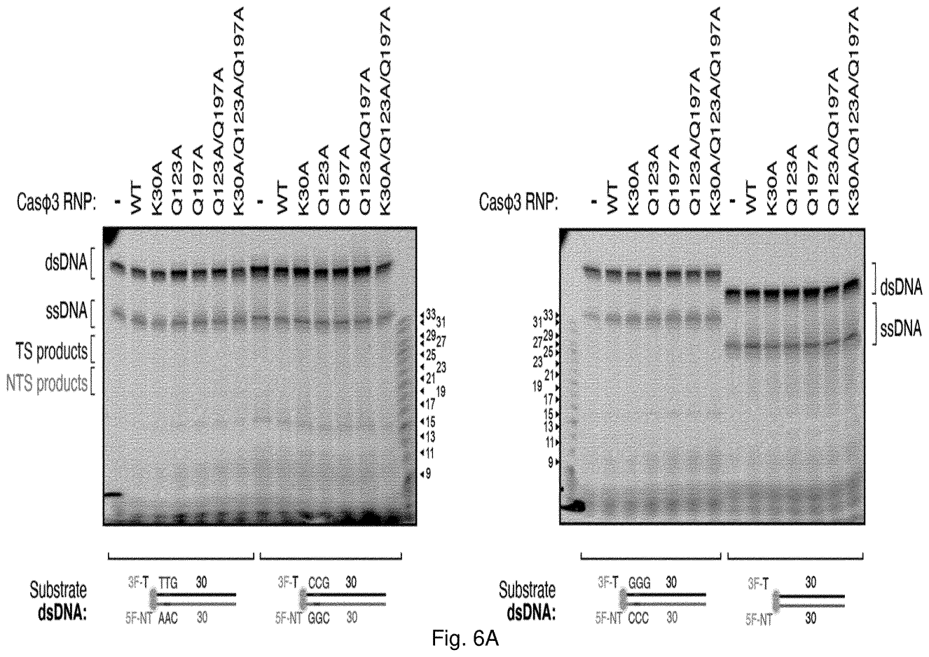

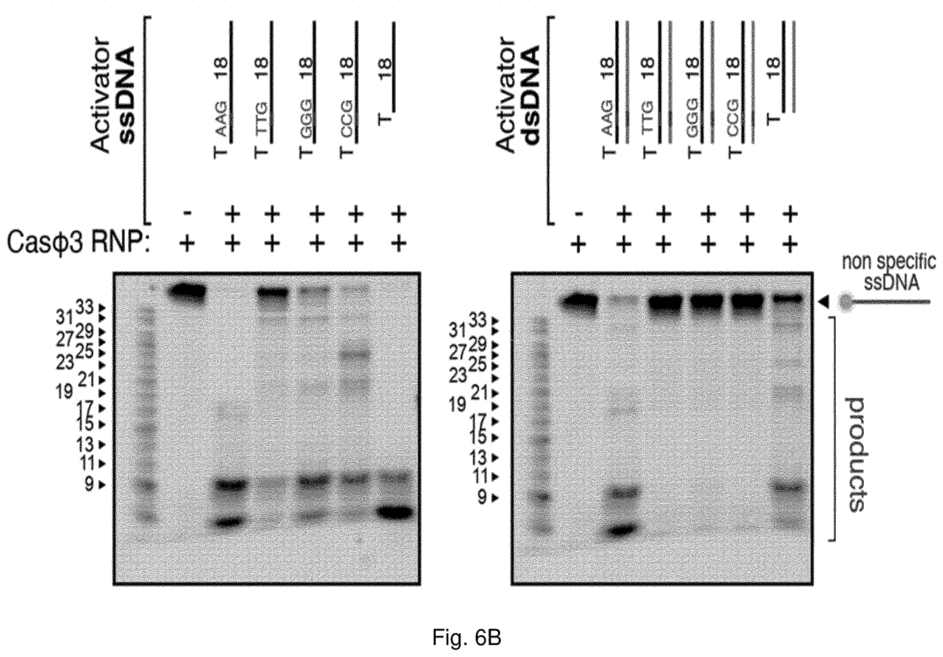

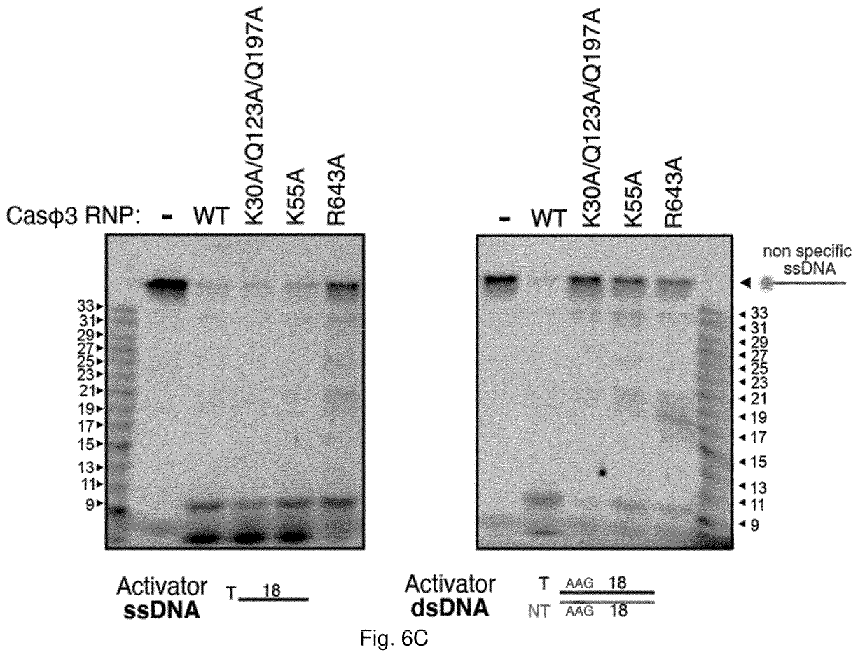

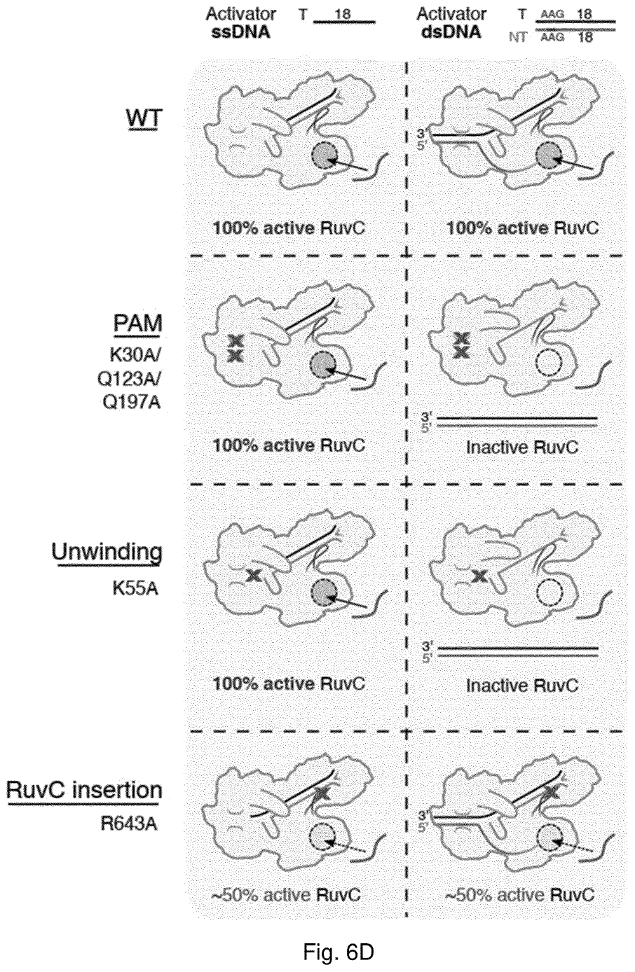

For specific dsDNA cleavage assays, FAM-labeled dsDNA substrates were incubated at 400 nM with 2 μM of CasΦ3 RNP in cleavage buffer (20 mM HEPES pH7.5, 160 mM KCl, 10% glycerol, 5 mM MgCl₂) for 2 h at 37° C., or as otherwise stated in the figure legends. For ion dependency assays 5 mM MgCl₂ was substituted by 5 mM Ethylenediaminetetraacetic acid (EDTA), CaCl₂, MnCl₂, FeSO₄, CoCl₂, NiSO₄, CuCl₂, ZnSO₄. For DNA saturation experiments 1 μM of CasΦ3 RNP was incubated with 0.5-8 uM of labelled dsDNA for 2 h at 37° C. For non-specific trans ssDNA cleavage assays (FIG. 5b-c, FIG. 6b-c), 0.4 UM FAM-labeled non-specific ssDNA substrate (i.e., not complementary to the crRNA) was incubated with 2 μM CasΦ3 RNP as described above, along with 0.1 μM of unlabeled activator ssDNA or dsDNA (complementary to the crRNA) in cleavage buffer for 1 h at 37° C. The reactions were stopped by adding equal volumes of stop buffer (8 M Urea, 100 mM EDTA at pH8) followed by incubation at 95° C. for 5 min. Cleavage products were resolved on 15% Novex TBE-Urea Gels (Invitrogen), run according to manufacturer's instructions. Gels were imaged using an Odyssey FC Imaging System (Li-Cor). Densitometric analysis of bands in gels was performed using ImageJ. The cleavage efficiency was calculated as the intensity of the bands corresponding to the products divided by the total intensity for the specific dsDNA cleavage assays, or as the depletion of signal of the non-cleaved product for non-specific ssDNA degradation assays.

### Sample Preparation for Cryo-EM

For the preparation of the Cryo-EM sample, Ni²⁺ was used as a catalytic ion instead of Mg²⁺ due to the higher yield obtained with this metal. CasΦ3 RNP was prepared as described before. 25 nmol of RNP and 37 nmol of unlabeled dsDNA substrate were incubated in 25 ml of MonoQ A buffer (20 mM HEPES pH7.5, 200 mM KCl, 1 mM NiSO₄, 0.5 mM TCEP) for 2 h at 20° C. to allow DNA cleavage. The product of the reaction was loaded in a MonoQ column equilibrated with MonoQ A buffer, and CasΦ3 R-loop complex was separated from the RNP and the unbound DNA substrate by a salt gradient elution using MonoQ B buffer (20 mM HEPES pH7.5, 2 M KCl, 1 mM NiSO₄, 0.5 mM TCEP). CasΦ3 R-loop eluted at 16-20% of MonoQ buffer B (~500 mM KCl). The R-loop complex was further purified from unbound DNA by SEC using a Superdex 200 Increase 10/300 GL column (Cytiva) equilibrated with MonoQ A buffer. The molecular weight of the complex and the sample homogeneity was estimated using a Refeyn One mass photometer (Refeyn), using 10-20 nM of protein diluted in MonoQ A buffer. 2.5 μL of freshly purified CasΦ3 R-loop complex (Absorbance_{260 nm} of ~1.6) was applied to UltrAuFoil 300 mesh R0.6/1.0 holey grids (Quantifoil), glow-discharged for 60 s at 10 mA (Leica EM ACE200), and plunge-frozen in liquid ethane (pre-cooled with liquid nitrogen) using a Vitrobot Mark IV (FEI, Thermo Fisher Scientific) using the next conditions: blotting time 3 s, 100% humidity and 4° C.

### CryoEM Data Collection and Processing

Movies were collected on Titan Krios G3 Cryo-TEM equipped with a TFS Falcon III camera operated at 300 keV in counting mode. Exposure 1.05 e/Å²/frame, in 40 frames and hence a final dose of 42 e/Å². The calibrated pixel size was 0.832 Å/px. All movies were pre-processed using WARP 1.0.9 (Tegunov et al., 2019). Motion correction was performed with a temporal resolution of 20 for the global motion and 5×5 spatial resolution for the local motion. We considered motion in the 45-3 Å range weighted with a B-factor of −500 Å². Only Micrographs displaying less than 5 Å intraframe motion were used. CTF estimation was performed using 5×5 patches in the 35-4 Å range. We selected micrographs with fitted defocus between 0.0 and 5.0 μm, and a resolution better than 5 Å. For the particle picking, the micrographs were masked, and particles were picked using a re-trained BoxNet deep convolutional neural network. This resulted in 3,504, 102 particles from 4,393 micrographs. Particles were extracted with a box size of 256×256 and a pixel size of 0.832 which were inverted and normalized before being imported into RELION 3.1 (Zivanov et al., 2018) for 2D classification. The selected 2D classes were imported in cryoSPARC 3.1.0 (Punjani et al., 2017) where they were 3D classified into four initial classes. The volume with the largest number of particles was 3D autorefined to an initial 2.61 Å resolution map. The conformational heterogeneity of the particles used in this volume was inspected through a 3D variability analysis job, and the two more divergent volumes were used as input for heterogeneous refinement. The 3D variability of the particles in the best volume was further analysed followed by heterogeneous refinement with four classes. The resulting four volumes were non-uniform refined to obtain maps at 2.7-3.3 Å resolution. The two best maps (2.7 and 2.9 Å resolution) represent the different conformational states of the complex that are discussed in the text. Sharpened and local resolution maps were calculated with PHENIX (Liebschner et al., 2019), and directional resolution anisotropy analysis were performed with the 3D-FSC server (Tan et al., 2017).

### Atomic Model Building and Refinement

An initial model containing the complete DNA and RNA sequence and ~50% of the protein sequence was built ab initio using map-to-model implemented in PHENIX (Liebschner et al., 2019). COOT (Emsley & Cowtan, 2004) was used to connect, extend and correct the protein fragments to generate a model covering ~70% of the protein sequence. The rest of the model was autobuilt by using buccaneer implemented in CCP-EM (Burnley et al., 2017), and subsequently corrected in COOT. The final model was obtained after several rounds of refinement using phenix.real_space_refine and manual inspection and correction in COOT. The final model covers 92% of the protein sequence, mainly lacking a C-terminal segment predicted to be unstructured. Map and molecular model images were created using ChimeraX (Goddard et al., 2018).

### Results

### CasΦ3/R-Loop Structure Determination

We reconstituted and characterized a functional CasΦ3-crRNA complex (FIG. 5) and determined the structure of the enzyme after severing a target dsDNA by cryo-EM (FIG. 1). Heterogeneous refinement resulted in several conformations of the complex. The predominant class yielded a map at a resolution of 2.7 Å, which was used to build the model of the CasΦ3/R-loop structure. The high flexibility observed in the second predominant class precluded building a complete model but revealed the flexible regions and the conformational heterogeneity of the complex. The CasΦ3/R-loop structure represents a snapshot of the endonuclease-product complex after substrate cleavage (FIG. 1c-e), revealing the critical residues for PAM recognition, target DNA unwinding and cleavage, and thereby providing detailed atomic information for the redesign of this novel family of genome editing tools.

### CasΦ3 Biochemical Characterisation

CasΦ3 generates an overhang of 9-11 nucleotides by cleaving a specific target DNA at different phosphodiester bonds (FIG. 1b, FIG. 5a). A collateral effect of its specific cleavage is the release of indiscriminate ssDNA degradation (Pausch et al., 2020), which is triggered by the T-strand provided as target dsDNA or as a ssDNA activator complementary to the crRNA (FIG. 5b-c). In both cases, indiscriminate CasΦ3 cleavage is unleashed when a minimal 12- to 13-nt crRNA-DNA duplex is assembled. The structure suggest that the differences observed with activators longer than 18-nt can be attributed to the presence of the R-loop disturbing the entrance of the unspecific ssDNA substrate in the catalytic site (FIG. 1d-e, FIG. 5d). The activity of the endonuclease was tested in the presence of Mg²⁺ and other divalent metal ions (FIG. 5e). The assay revealed that CasΦ3 supports catalysis in the presence of Mn²⁺, Fe²⁺, Co²⁺, and Ni²⁺ resulting in different cleavage patterns. CasΦ3 cleavage activity was saturated when the endonuclease/target-DNA ratio was nearly equimolar, suggesting the slow dissociation of the enzyme from the PAM-proximal cleavage product, as observed in other RNA-guided nucleases (Stella et al., 2017a and Sternberg et al., 2014) (FIG. 5f). In addition, removing the last 39 residues of the C-terminus, which were not visualized in the structure, decreased CasΦ3 activity. However, the enzyme conserved a substantial catalytic activity, suggesting that Caso family members can be further miniaturized (FIG. 5g-h).

### Overall Structure of the CasΦ3/R-Loop Complex

The CasΦ3/R-loop complex does not present the classical bilobal architecture observed in other type V effector complexes. The R-loop displays a T shape with the crRNA/DNA hybrid and the crRNA handle forming the horizontal and vertical bars, and the protein domains wrapping around the nucleic acids (FIG. 1d-e). The handle of the crRNA is stabilized by the strictly conserved R338 which interacts with C-1 and U-18 and the neighbouring non-Watson-Crick base pair interaction between G-17 and A-2. The PAM-distal and PAM-proximal regions of the heteroduplex are recognized by the N- and C-terminal regions of the polypeptide (FIG. 1d-e), which are connected by a 15-residue loop (380-395). Each region comprises around half of the size of the protein and they are separated by the long handle of the crRNA on the T-shape assembly. The N-terminal region comprises the T-strand and NT-strand PAM interacting domains (TPID, NPID) and the RNA-handle binding domain (RBD), while the C-terminal consists of the catalytic RuvC and the stop (STP) domains (FIG. 1a). The RuvC domain is split into RuvC-I and RuvC-II by the insertion of the STP domain, which is connected to the catalytic domain by two long bridge helices, BH-I and BH-II. Additionally, the RuvC-II subdomain presents a characteristic insertion, which is conserved in all the known members of the CasΦ family except CasΦ7 (FIG. 1). This N- and C-terminal physical separation is also functional, as the RNP assembly, PAM recognition and unwinding reside in the N-terminal region, while the crRNA/T-strand hybrid assembly and catalysis of the target DNA are performed by the C-terminal section of the polypeptide. Therefore, the PAM binding site is ~55 Å away from the RuvC nuclease active site.

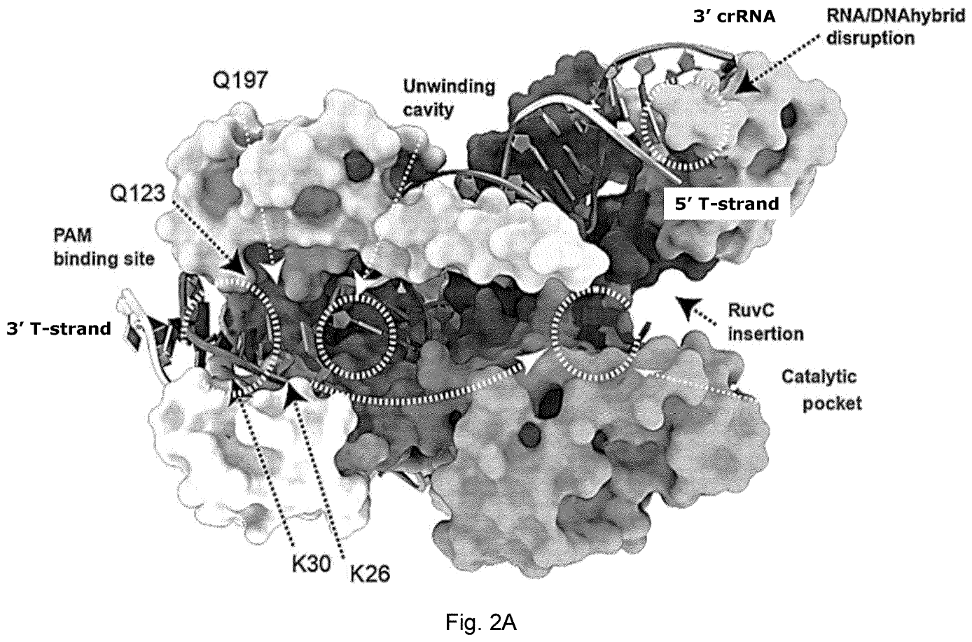

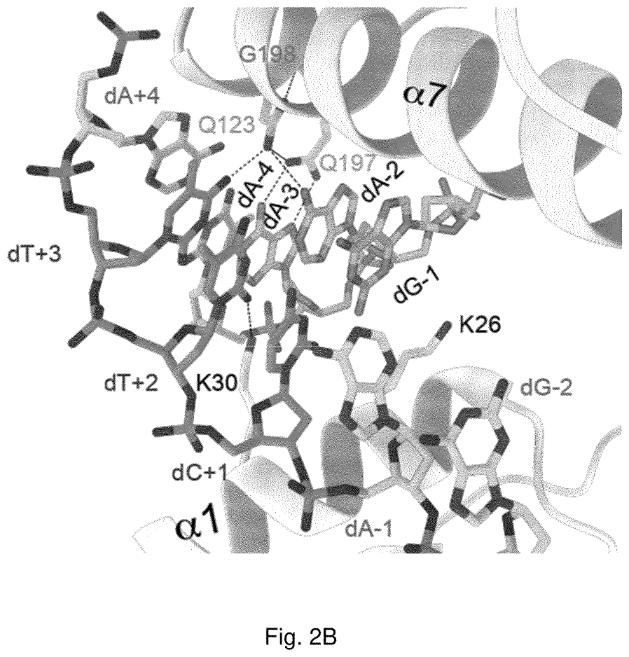

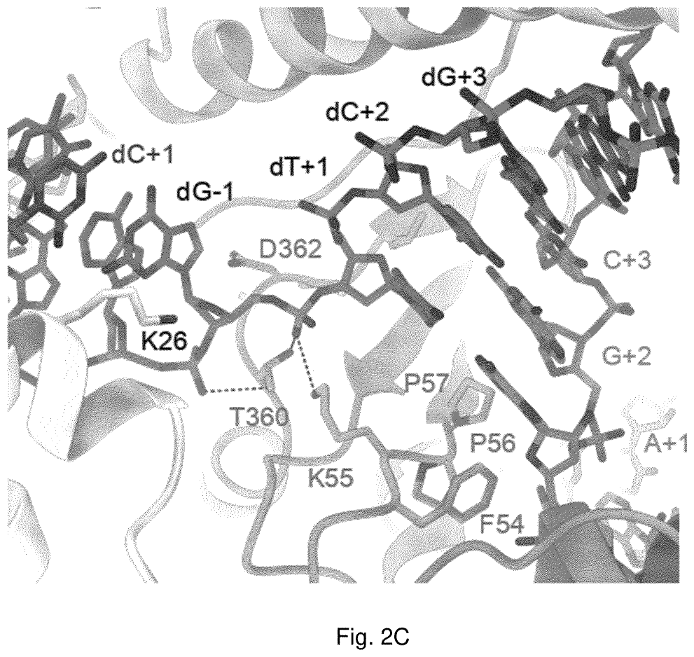

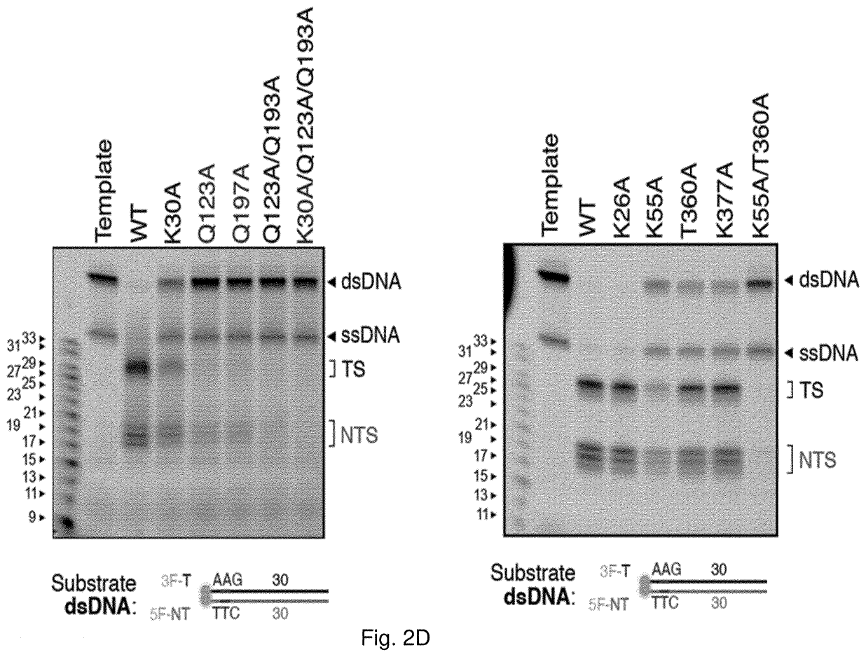

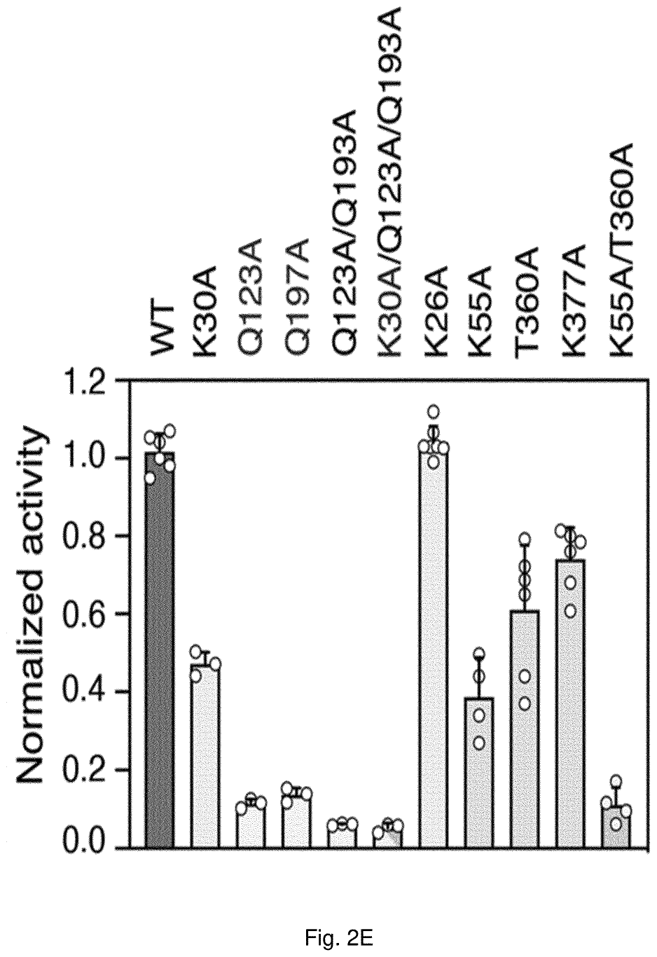

The target DNA cleavage yields a triple strand R-loop with the T-strand hybridized to the crRNA (FIG. 1b, d), while the dissociated PAM NT-strand is directed towards the RuvC catalytic pocket (FIG. 2a). The NT-strand nucleotides-1 to -2 upstream of the PAM were built in the density but the high flexibility on the distal end of the NT-strand precluded visualization of the rest of the nucleotides, as shown for Cas9 (Jiang et al., 2016) and Cas12a (Stella et al., 2017). Nevertheless, the backbone of the NT-strand is observed at low contour level in the cryo-EM maps, suggesting the path followed by the DNA to the RuvC catalytic pocket (FIG. 2a). Interestingly, two nucleotides, modelled as purines, were observed in the RuvC pocket in complex with Ni²⁺ as a by-product of the phosphodiester hydrolysis (FIG. 1c-e, 2a). To determine to which strand these nucleotides belong, we performed a binding assay after cleavage with different labelled target DNA, revealing that these nucleotides originate from the NT-strand.

### PAM Recognition

PAM recognition is an important aspect of DNA targeting by CRISPR-Cas nucleases, as it is a prerequisite for target DNA identification, strand separation and crRNA-target-DNA heteroduplex formation (Anders et al., 2014) before cleavage. CasΦ3 is reported to recognize a 5′-TTN-3′ PAM sequence in the NT-strand (Pausch et al., 2020). Our structure shows that PAM recognition in CasΦ3 is achieved by a combination of interactions in both strands by the TPID and NPID domains (FIG. 2b). The positively charged side of helix a1 (S21 to A34) in the NPID is inserted in the minor groove at an angle of 45° with respect to the dsDNA longitudinal axis, thus facilitating the unwinding of the dsDNA. Two conserved lysines, K26 and K30, interact with the NT-strand. K30 makes specific contacts with dT+2, while K26 is placed inside the dsDNA to disrupt Watson-Crick base coupling, displacing the NT-strand and promoting separation (FIG. 2b-c). On the other side of the PAM recognition cleft, Q123 in the TPID builds an intricate network of polar interaction with dA-3, dA-2 in the T- and the dT+3 in the NT-strand (FIG. 2b). The neighbouring G198 amide contacts the carbonyl of Q123, anchoring the side chain in a conformation favouring the contacts with these bases. In addition, the side chain of Q197 interacts with Q123 and hydrogen bonds with dA-3.

The Q123A and Q197A mutations present ~90% activity reduction, while the K30A mutant reduces cleavage ~55%. The triple mutant activity is similar to the Q123A/Q197A mutant, indicating the pivotal role of the glutamines in PAM recognition, as the addition of the K30A mutation does not display a further reduction (FIG. 2d-e). The K26A mutant activity is not affected, suggesting that the insertion of the a1 helix is sufficient to unzip the dsDNA. All the mutants involved in PAM recognition do not change the cleavage pattern of the dsDNA target (FIG. 2d-e). Both the wild type and the mutants did not cleave target dsDNAs with different PAM sequences or in the absence of PAM, underscoring the selectivity of the PAM interaction network formed by Q123A, Q197A and K30A (FIG. 6a). In addition, we observed that the unspecific ssDNA catalysis is also fully activated in the presence of dsDNA containing the PAM, thus, suggesting that after PAM recognition crRNA/DNA hybrid assembly activates catalysis (FIG. 6b). Finally, to assess the role of the PAM complementary bases in the T-strand, we triggered the unspecific activity of CasΦ3 using ssDNAs activators mimicking the T-strand with different PAM sequences. The assay showed that the PAM complementary 3′-AAG-5′ sequence and an activator without PAM, fully released phosphodiester hydrolysis, while other PAMs promoted activation to different levels. This experiment suggests that the assembly of the proper hybrid unleashes the catalytic activity, while activators containing regions that partially hybridize with the crRNA display lower cleavage (FIG. 6b).

Collectively, our analysis suggests that the well-conserved Q123 and Q197 residues, which interact with the PAM in the major groove of the target DNA, play an essential role in recognition. The direct base readout in the PAM region of Caso nucleases combine interactions of the TPID and NPID with both strands of the target DNA. However, the interactions of the TPID with the T-strand seem to have an important role in PAM discrimination. This is a singular property of the CasΦ family, as other CRISPR-Cas nucleases perform PAM scanning by interacting preferentially with the NT-strand (Jiang et al., 2017 and Stella et al., 2017b) 17,18

### Target DNA Unwinding

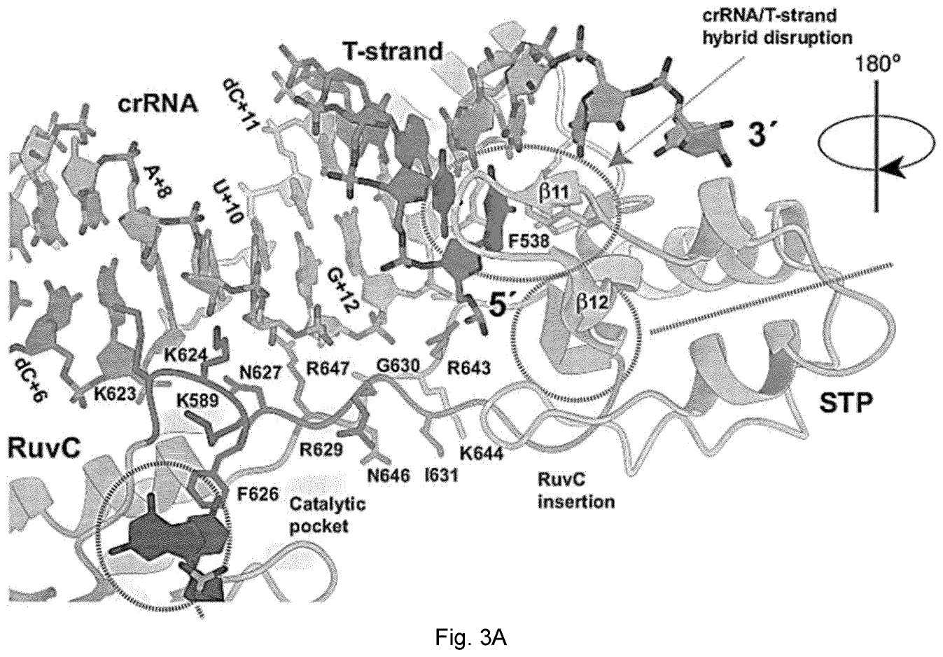

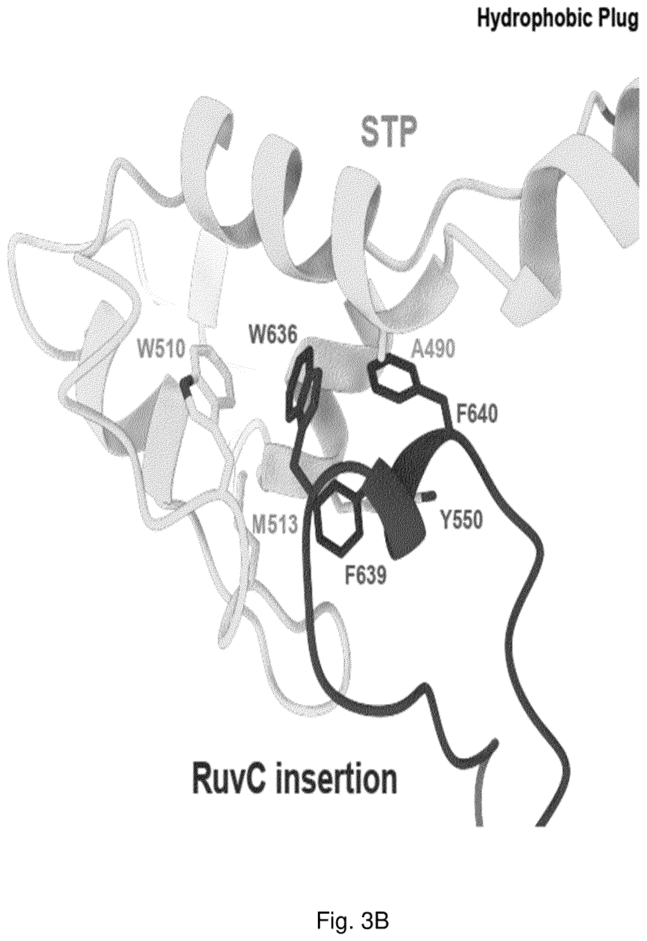

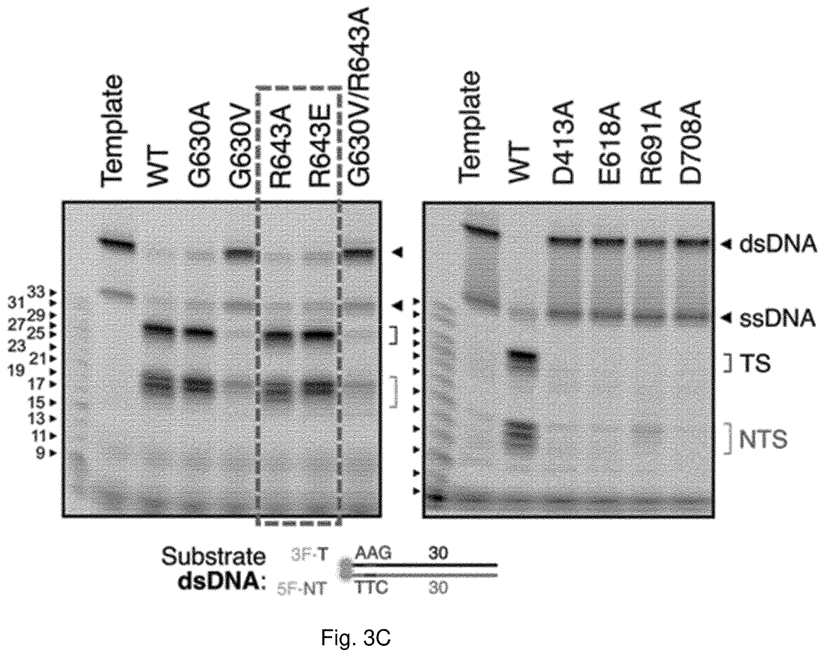

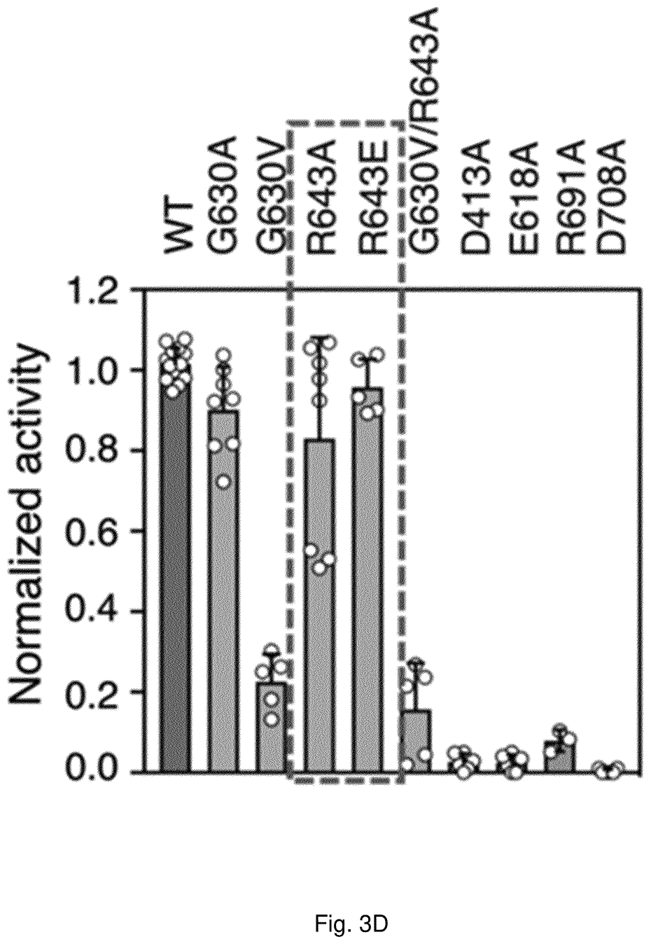

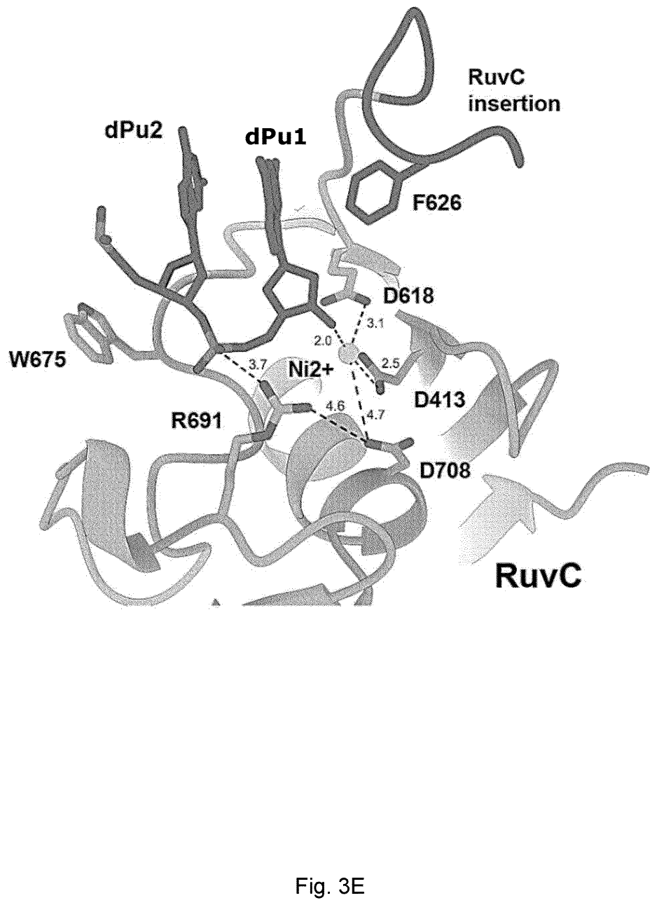

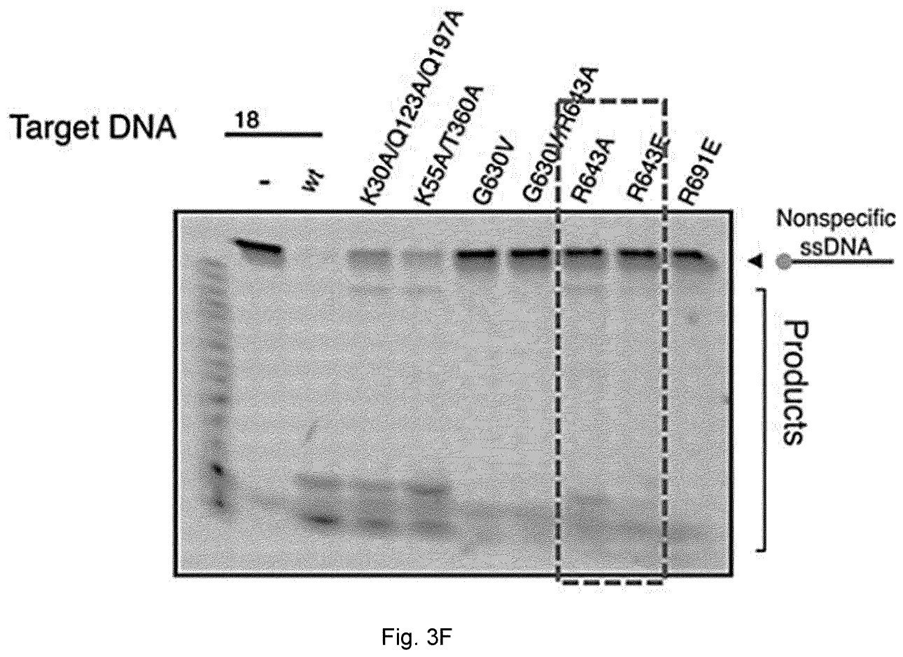

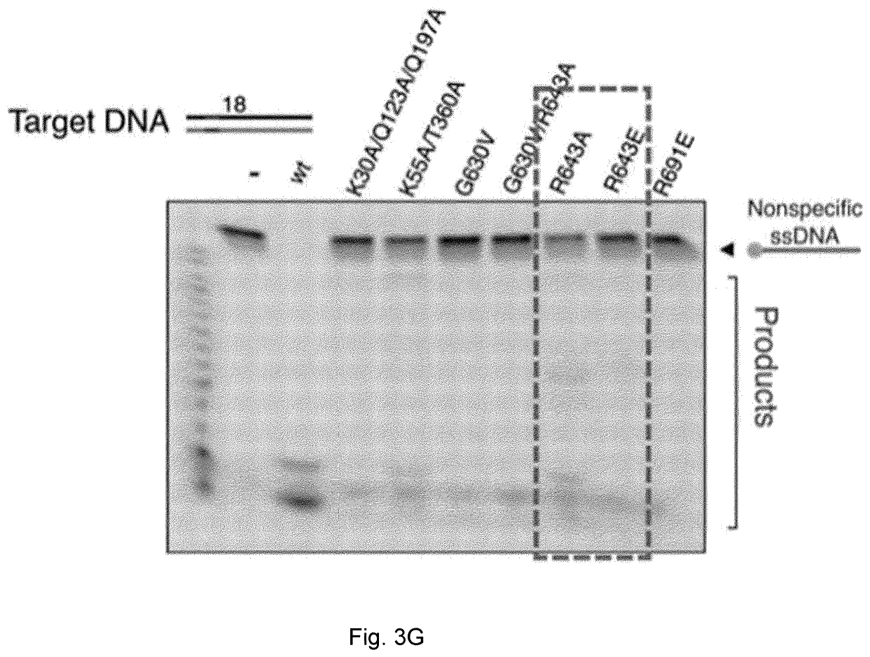

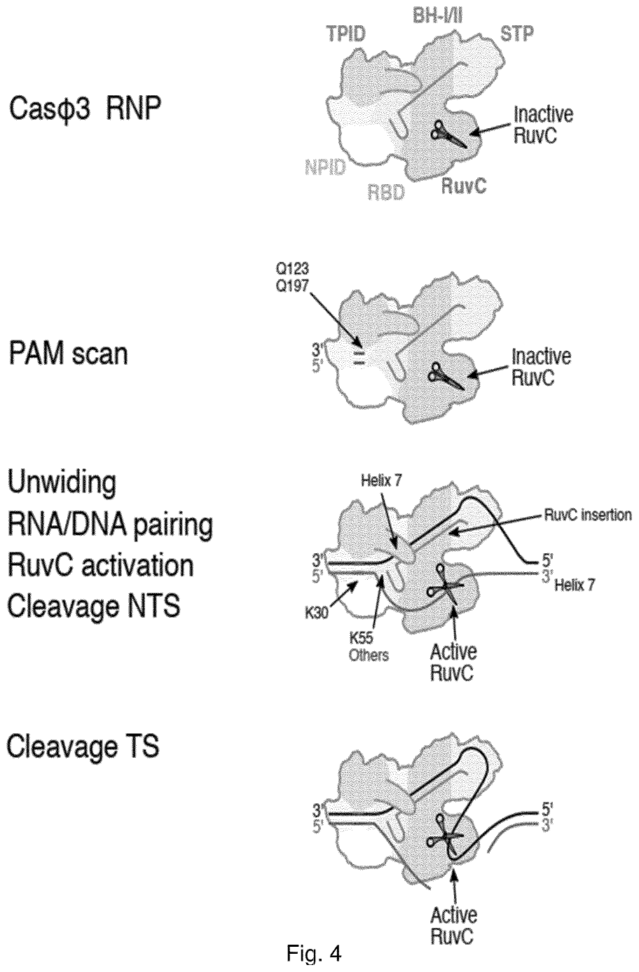

Overlaying with the first uncoupled base pair upstream the PAM, the TPID, NPID and the antiparallel b-sheet composed of the b1, b6 and b7 strands of the RBD domain, build a cavity where unwinding and the initial crRNA/T-strand hybridisation occurs (FIG. 2c). This cavity is flanked on the C-terminal region by the BH-I helix and the RuvC domain. The well-conserved F54, K55, P56, P57, P363, T360, G361, D362 and V364 organize the cavity combining acidic and hydrophobic residues facilitating the Watson-Crick base pairing of dT+1 and A+1 in the T-strand and the seed of the crRNA (FIG. 2c). In addition, the backbone phosphate group of dG-1 is recognized by the side chain of the T360, K55 and the main chain of Y376. This interaction results in the rotation of the phosphate group (FIG. 2c), facilitating base pairing between dT+1 and A+1 in the crRNA as observed in Cas9 (Jiang et al., 2015) and Cas12a complexes (Stella et al., 2017a, Stella et al., 2018, Swarts and Jinek, 2019, Swarts et al., 2017 and Yamano et al., 2016). The neighbouring K377A mutation led to ~20% decrease in the activity, but the T360A and the K55A mutations displayed a reduction of 50% and 60%, highlighting the importance of these residues for phosphate inversion and hybrid formation (FIG. 2d-e). The long helix a7 in the TPID directs the crRNA/T-strand hybrid into the “nest” formed by the BH-I and II helices and the RuvC insertion, and detaches the hybrid from the NT-strand preventing a possible reannealing of the target DNA. The area where the hybrid rests is flanked by the catalytic RuvC and STP domains, which disrupts the crRNA/T-strand hybrid as a vessel bulb bow (FIG. 3a). An antiparallel b-sheet formed by b11 and b12 splits the Watson-Crick base coupling after the dG-17: C+17 pair; thus, limiting the hybrid length to 17 nucleotides in agreement with cleavage experiments testing the efficiency of the spacer length (Pausch et al., 2020). The aromatic ring of F538 in b11 initiates the hybrid unzipping (FIG. 3a). The 3′-phosphate of the crRNA is guided to the back side of the domain, where C+17 and U+18 are accommodated by a combination of basic (R535, R547) and hydrophobic residues (M500, L555), and the 5′-phosphate of the T-strand is directed to the other side of the protein where the RuvC catalytic pocket is located.

### Catalytic Activation

The RuvC insertion runs alongside the crRNA strand of the hybrid, making multiple contacts with its phosphate backbone from U+9 to G+13, and the turn at the tip of the insertion is anchored in the back side of the STP domain by hydrophobic interactions (FIG. 3b). This arrangement and the activity assays (FIG. 5b-c, FIG. 6c-d), suggest that the assembly of the crRNA/DNA hybrid could trigger conformational changes in the RuvC insertion that activate catalysis by making the active pocket available for the ssDNA substrate. The monitoring of the unspecific cleavage of ssDNA substrate using activators of different length (FIG. 5b-c), shows that the unspecific activity of CasΦ3 is fully released when the activator's length allows the formation of a 12-nt crRNA/DNA hybrid or longer, supporting the notion that a certain hybrid length is needed to activate catalysis. The conserved G630 and R643 are key residues, as they arrange a network of polar interactions with the phosphate of G+12, resulting in a special arrangement of the connections joining the hydrophobic “plug” composed by the conserved W636, F639 and F640 residues in the tip of the insertion (FIG. 3a-b). We hypothesize that the assembly of the hybrid would promote the observed conformation of the RuvC insertion, which is anchored by the plug in the cleft of the STP domain composed by A490, W510, M513 (FIG. 3b). The stabilisation of this conformation by the hybrid would pull the STP domain towards the catalytic site, placing the T-strand in the active site with the proper 5′-3 polarity. Mutations in the hydrophobic plug and STP cleft residues rendered CasΦ3 insoluble, highlighting the importance of this conserved interaction in the Caso family.

To test the activation hypothesis, we analysed substitutions in G630 and R643. The G630A mutation exhibited a minor activity decrease ~10% (FIG. 3c-d), in agreement with the G630 contribution to the polar network through its main chain. However, G630V displayed a strong reduction, suggesting that a bulkier side chain affects the interaction with the phosphate, and supporting the important role of the conserved G630 in monitoring crRNA/DNA assembly. Interestingly, the reversed polarity mutant R643E presented a minimal cleavage reduction of the target DNA (FIG. 3c-d), but its indiscriminate ssDNA degradation activity showed ~100% reduction, likewise G630V (FIG. 6c-d); thereby showing that substitutions in the RuvC insertion can modify Cas12j family cleavage.

In addition, all the PAM and unwinding mutants display full indiscriminate ssDNA activity when the same assay was performed using a ssDNA activator lacking the PAM. This activator would skip recognition and unwinding, thus hybridising with the crRNA and triggering activity. However, when the PAM is present in the target dsDNA the variants displayed a minimal activity, as their PAM recognition and unwinding are compromised, in agreement with their specific dsDNA cleavage activity (FIG. 2 d-e). These results support the proposed model, as the PAM and unwinding mutants would skip recognition and unwinding when activated with ssDNA, thus hybridising with the crRNA and triggering the nuclease activity.

Therefore, PAM recognition, DNA unwinding and activation are linked in the presence of a target dsDNA, while catalytic activation can omit PAM recognition if a suitable ssDNA is provided. Furthermore, mutations in the RuvC insertion do not only affect the enzyme activity, they can dissociate the indiscriminate ssDNA activity from the specific target dsDNA cleavage and change its pattern as observed in the case of the G630V and R643E mutants.

### DNA Cleavage

The RuvC domain of CasΦ nucleases belong to the retroviral integrase superfamily that displays a characteristic RNaseH fold. The two nucleotides from the NT-strand in the catalytic CasΦ3 pocket are associated with the conserved E618 and D413 (FIG. 3e). The density did not allow base identification, and either dA or dG could be modelled. We built two guanines with a 5′-3′ polarity and a Ni²⁺ ion (Methods), in agreement with the number of nucleotides in the cleavage products and the purine rich sequence in that position (FIG. 1b, 3e). Therefore, the length of the DNA after DSB generation could permit that the cleaved NT-strand remains associated with the catalytic centre and may disturb the entrance of the T-strand delaying its catalysis, as previously observed (Pausch et al., 2020) (FIG. 5g). A second metal atom, modelled as Zn, is coordinated by 4 conserved cysteines, similarly to Cas12f (Takeda et al., 2021) and Cas12g (Li et al., 2021). This section of RuvC includes the conserved R691 3.7 Å away from the dinucleotide. This residue could facilitate the positioning of the phosphodiester backbone in the catalytic pocket (FIG. 3e). However, the rest of this region is different to the target nucleic acid-binding (TNB) domain in Cas12f and Cas12g (also known as the Nuc domain for Cas12a and Cas12b and the target-strand loading domain for Cas12e), as it displays a different structure that does not contain the helical regulatory lid motif.

RuvC domains introduce 5′-phosphorylated cuts and involve three acidic amino acids (Nowotny, 2009) and two divalent metal ions (Steitz & Steitz, 1993). The E618 and D413 carboxylate amino acids are important catalytic residues, and the E618A and D413A mutations abolish CasΦ3 activity (FIG. 3c-e). Both residues are predicted to coordinate the metal ions that activate the nucleophile and stabilize the transition state and the leaving group. In our structure, E618 and D413 coordinate the metal and the backbone of the dinucleotide (FIG. 3e). The side chain of D708, which is predicted to act as the third catalytic residue, is not observed due to electron irradiation (Bartesaghi et al., 2014). This active-site residue has been shown less critical than the other carboxylates in other RuvC domains, and substitutions of this amino acid to Asn or His lead to only partial loss of cleavage (Chapados et al., 2001 and Kanaya, 1998). However, the D708A mutation abrogates activity (FIG. 3c-e). Structural comparisons using DALI with other RuvC domains, including CRISPR-Cas proteins, support a two metal ion mechanism. Interestingly, we cannot observe differences with the RuvCs of CasΦ1 and 2 that could explain why CasΦ3 is unable to cleave, and thereby process, its own crRNA, as the sequence homology in this domain is high within the CasΦ family.

### Sequence Overview

| | | Organism |
|---|---|---|
| SEQ | | and optionally |
| ID NO: | Description | accession number |
| 1 | Casφ-1 (protein) | Biggiephage |
| 2 | Casφ-2 (protein) | Biggiephage |
| 3 | Casφ-3 (protein) | Biggiephage |
| 4 | Casφ-4 (protein) | Biggiephage |
| 5 | Casφ-5 (protein) | Biggiephage |
| 6 | Casφ-6 (protein) | Biggiephage |
| 7 | Casφ-7 (protein) | Biggiephage |
| 8 | Casφ-8 (protein) | Biggiephage |
| 9 | Casφ-9 (protein) | Biggiephage |
| 10 | Casφ-10 (protein) | Biggiephage |
| 11 | Casφ-1-bacteria optimized (DNA) | Artificial |
| 12 | Casφ-2-bacteria optimized (DNA) | Artificial |
| 13 | Casφ-3-bacteria optimized (DNA) | Artificial |
| 14 | Casφ-4-bacteria optimized (DNA) | Artificial |
| 15 | Casφ-5-bacteria optimized (DNA) | Artificial |
| 16 | Casφ-6-bacteria optimized (DNA) | Artificial |
| 17 | Casφ-7-bacteria optimized (DNA) | Artificial |
| 18 | Casφ-8-bacteria optimized (DNA) | Artificial |
| 19 | Casφ-9-bacteria optimized (DNA) | Artificial |
| 20 | Casφ-10-bacteria optimized (DNA) | Artificial |
| 21 | Casφ-1-human optimized (DNA) | Artificial |
| 22 | Casφ-2-human optimized (DNA) | Artificial |
| 23 | Casφ-3-human optimized (DNA) | Artificial |
| 24 | Casφ-4-human optimized (DNA) | Artificial |
| 25 | Casφ-5-human optimized (DNA) | Artificial |
| 26 | Casφ-6-human optimized (DNA) | Artificial |
| 27 | Casφ-7-human optimized (DNA) | Artificial |
| 28 | Casφ-8-human optimized (DNA) | Artificial |
| 29 | Casφ-9-human optimized (DNA) | Artificial |
| 30 | Casφ-10-human optimized (DNA) | Artificial |
| 31 | Casφ-3-ΔCT (protein) | Artificial |
| 32 | Casφ-3-ΔCT (DNA bact. opt.) | Artificial |
| 33 | Casφ-3-ΔCT (DNA human opt.) | Artificial |
| 34 | Casφ-1 crRNA constant region (RNA) | Artificial |
| 35 | Casφ-2 crRNA constant region (RNA) | Artificial |
| 36 | Casφ-3 crRNA constant region (RNA) | Artificial |
| 37 | Example crRNA (figure 1B) | Artificial |
| 38 | Example template | Artificial |
| | strand sequence (figure 1B) | |
| 39 | Example non-template | Artificial |
| | strand sequence (figure 1B) | |

### REFERENCES

Anders, C., Niewoehner, O., Duerst, A. & Jinek, M. Structural basis of PAM-dependent target DNA recognition by the Cas9 endonuclease. Nature 513, 569-573, doi: 10.1038/nature 13579 (2014).
Bartesaghi, A., Matthies, D., Banerjee, S., Merk, A. & Subramaniam, S. Structure of β-galactosidase at 3.2-Å resolution obtained by cryo-electron microscopy. Proceedings of the National Academy of Sciences 111, 11709, doi: 10.1073/pnas. 1402809111 (2014).
Burnley, T., Palmer, C. M. & Winn, M. Recent developments in the CCP-EM software suite. Acta Crystallographica Section D 73, 469-477, doi: doi: 10.1107/S2059798317007859 (2017).
Chapados, B. R. et al. Structural biochemistry of a type 2 RNase H: RNA primer recognition and removal during DNA replication. J Mol Biol 307, 541-556, doi: 10.1006/jmbi.2001.4494 (2001).
Emsley, P. & Cowtan, K. Coot: model-building tools for molecular graphics. Acta Crystallogr D Biol Crystallogr 60, 2126-2132, doi: 10.1107/S0907444904019158 (2004).
Goddard, T. D. et al. UCSF ChimeraX: Meeting modern challenges in visualization and analysis. Protein Sci 27, 14-25, doi: 10.1002/pro.3235 (2018).
Jiang, F., Zhou, K., Ma, L., Gressel, S. & Doudna, J. A. STRUCTURAL BIOLOGY. A Cas9-guide RNA complex preorganized for target DNA recognition. Science 348, 1477-1481, doi: 10.1126/science.aab1452 (2015).
Jiang, F. et al. Structures of a CRISPR-Cas9 R-loop complex primed for DNA cleavage. Science 351, 867-871, doi: 10.1126/science.aad8282 (2016).
Jiang, F. & Doudna, J. A. CRISPR-Cas9 Structures and Mechanisms. Annual Review of Biophysics 46, 505-529, doi: 10.1146/annurev-biophys-062215-010822 (2017).
Kanaya, S. Enzymatic activity and protein stability of E. coli ribonuclease HI. Ribonucleases H., 1-38 (1998).
Li, Z., Zhang, H., Xiao, R., Han, R. & Chang, L. Cryo-EM structure of the RNA-guided ribonuclease Cas12g. Nature Chemical Biology 17, 387-393, doi: 10.1038/s41589-020-00721-2 (2021).
Liebschner, D. et al. Macromolecular structure determination using X-rays, neutrons and electrons: recent developments in Phenix. Acta Crystallogr D Struct Biol 75, 861-877, doi: 10.1107/S2059798319011471 (2019).
Makarova, K. S., and Koonin, E. V. (2015). Annotation and Classification of CRISPR-Cas Systems. Methods Mol Biol 1311, 47-75.
Nowotny, M. Retroviral integrase superfamily: the structural perspective. EMBO Rep 10, 144-151, doi: 10.1038/embor.2008.256 (2009).
Pausch, P. et al. CRISPR-CasPhi from huge phages is a hypercompact genome editor. Science 369, 333-337, doi: 10.1126/science.abb1400 (2020).
Punjani, A., Rubinstein, J. L., Fleet, D. J. & Brubaker, M. A. cryoSPARC: algorithms for rapid unsupervised cryo-EM structure determination. Nature Methods 14, 290-296, doi: 10.1038/nmeth.4169 (2017).
Steitz, T. A. & Steitz, J. A. A general two-metal-ion mechanism for catalytic RNA. Proc Natl Acad Sci USA 90, 6498-6502, doi: 10.1073/pnas.90.14.6498 (1993).
Stella, S., Alcon, P. & Montoya, G. Structure of the Cpf1 endonuclease R-loop complex after target DNA cleavage. Nature 546, 559-563, doi: 10.1038/nature22398 (2017a).
Stella, S., Alcon, P. & Montoya, G. Class 2 CRISPR-Cas RNA-guided endonucleases: Swiss Army knives of genome editing. Nat Struct Mol Biol 24, 882-892, doi: 10.1038/nsmb.3486 (2017b).
Stella, S. et al. Conformational Activation Promotes CRISPR-Cas12a Catalysis and Resetting of the Endonuclease Activity. Cell 175, 1856-1871 e1821, doi: 10.1016/j.cell.2018.10.045 (2018).
Sternberg, S. H., Redding, S., Jinek, M., Greene, E. C. & Doudna, J. A. DNA interrogation by the CRISPR RNA-guided endonuclease Cas9. Nature 507, 62-67, doi: 10.1038/nature13011 (2014).
Swarts, D. C. & Jinek, M. Mechanistic Insights into the cis- and trans-Acting DNase Activities of Cas12a. Mol Cell 73, 589-600 e584, doi: 10.1016/j.molcel.2018.11.021 (2019).
Swarts, D. C., van der Oost, J. & Jinek, M. Structural Basis for Guide RNA Processing and Seed-Dependent DNA Targeting by CRISPR-Cas12a. Mol Cell 66, 221-233 e224, doi: 10.1016/j.molcel.2017.03.016 (2017).
Takeda, S. N. et al. Structure of the miniature type V-F CRISPR-Cas effector enzyme. Mol Cell 81, 558-570.e553, doi: 10.1016/j.molcel.2020.11.035 (2021).
Tan, Y. Z. et al. Addressing preferred specimen orientation in single-particle cryo-EM through tilting. Nat Methods 14, 793-796, doi: 10.1038/nmeth.4347 (2017).
Tegunov, D. & Cramer, P. Real-time cryo-electron microscopy data preprocessing with Warp. Nature Methods 16, 1146-1152, doi: 10.1038/s41592-019-0580-y (2019).
Yamano, T. et al. Crystal Structure of Cpf1 in Complex with Guide RNA and Target DNA. Cell 165, 949-962, doi: 10.1016/j.cell.2016.04.003 (2016).
Zivanov, J. et al. New tools for automated high-resolution cryo-EM structure determination in RELION-3. Elife 7, doi: 10.7554/eLife.42166 (2018).

### ITEMS

1. A mutant Cas12j endonuclease, such as a mutant Caso-3 or an orthologue thereof, comprising a polypeptide sequence having at least 95% sequence identity to:
   i) the sequence corresponding to residues 1 to 20, 36 to 97, 104 to 119, 151 to 179, 204 to 379, 396 to 619, 651 to 679, and 701 to 726 of SEQ ID NO: 3, wherein said polypeptide sequence further comprises:
      a. at least one amino acid mutation in a first region of the NPID domain corresponding to residues 21 to 35 of SEQ ID NO: 3, wherein each mutation independently is an amino acid substitution, insertion or deletion; and/or
      b. at least one amino acid mutation in a first region of the TPID domain corresponding to residues 98 to 103 of SEQ ID NO: 3, wherein each mutation independently is an amino acid substitution, insertion or deletion; and/or
      c. at least one amino acid mutation in a second region of the TPID domain corresponding to residues 120 to 150 of SEQ ID NO: 3, wherein each mutation independently is an amino acid substitution, insertion or deletion; and/or
      d. at least one amino acid mutation in a third region of the TPID domain or in a first region of the RBD domain corresponding to residues 180 to 203 of SEQ ID NO: 3, wherein each mutation independently is an amino acid substitution, insertion or deletion; and/or
      e. at least one amino acid mutation in a second region of the RBD domain or in a first region of the RuvC-I domain corresponding to residues 380 to 395 of SEQ ID NO: 3, wherein each mutation independently is an amino acid substitution, insertion or deletion; and/or
      f. at least one amino acid mutation in a first region of the RuvC-II domain corresponding to residues 620 to 650 of SEQ ID NO: 3, wherein each mutation independently is an amino acid substitution, insertion or deletion; and/or
      g. at least one amino acid mutation in a second region of the RuvC-II domain corresponding to residues 680 to 700 of SEQ ID NO: 3, wherein each mutation independently is an amino acid substitution, insertion or deletion; and/or
      h. at least one amino acid mutation in a third region of the RuvC-II domain corresponding to residues 726 to 766 of SEQ ID NO: 3, wherein each mutation independently is an amino acid substitution, insertion or deletion;
   and/or
   ii) SEQ ID NO: 3, wherein said polypeptide sequence comprises at least one amino acid substitution in a position selected from the positions corresponding to residues 26, 30, 54, 55, 123, 197, 355, 360, 413, 618, 625, 626, 630, 643, 673, 675, 676, 680, 683, 691, 698, 701 and 708 of SEQ ID NO: 3.
2. The mutant Cas12j endonuclease or orthologue thereof according to item 1, wherein said mutant endonuclease comprises a polypeptide sequence having at least 95% sequence identity to the sequence corresponding to residues 1 to 726 of SEQ ID NO: 3, wherein said polypeptide sequence further comprises a C-terminal deletion of the sequence corresponding to residues 727 to 766 of SEQ ID NO: 3, such as wherein the endonuclease comprises or consists of a polypeptide sequence having at least 95% sequence identity to SEQ ID NO: 31.
3. The mutant Cas12j endonuclease or orthologue thereof according to any one of the preceding items, wherein the mutant endonuclease has one or more altered activities compared to the wild type endonuclease, said activity being selected from the group consisting of double-stranded cleavage of a target nucleic acid sequence, single-stranded cleavage of a target nucleic acid sequence and target nucleic acid recognition.
4. The mutant Cas12j endonuclease or orthologue thereof according to any one of the preceding items, wherein the endonuclease is comprised in a medium comprising divalent nickel (Ni²⁺), divalent manganese (Mn²⁺) and/or divalent copper (Co²⁺).
5. A polynucleotide encoding the mutant Cas12j endonuclease or orthologue thereof according to any one of the preceding items.
6. A recombinant vector comprising a polynucleotide according to item 5, or a nucleic acid sequence encoding a mutant Cas12j endonuclease or orthologue thereof according to any one of items 1 to 4.
7. A cell capable of expressing the mutant Cas12j endonuclease or orthologue thereof according to any one of items 1 to 4, the polynucleotide according item 5, or the recombinant vector according to item 6.
8. A system for expression of a crRNA-Cas12j complex comprising
   a. a polynucleotide according to item 5, or a recombinant vector according to item 6 comprising a polynucleotide encoding a mutant Cas12j endonuclease or orthologue thereof; and
   b. a polynucleotide or a recombinant vector comprising a polynucleotide encoding a guide RNA (crRNA), optionally operably linked to a promoter; and
   c. optionally, a cell for expression of the polynucleotide or the recombinant vector of a. and b.
9. Use of a crRNA-Cas12j complex in a method for introducing a nucleic acid break in a first target nucleic acid, wherein:
   a. a mutant Cas12j endonuclease or orthologue thereof is contacted with a guide RNA (crRNA), thereby obtaining a crRNA-Cas12j complex capable of recognizing a second target nucleic acid, the second target nucleic acid comprising a protospacer adjacent motif (PAM), and wherein the Cas12j endonuclease or orthologue thereof is according to any one of items 1 to 4;
   b. the crRNA-Cas12j complex is contacted with the first target nucleic acid; whereby a nucleic acid break is made in the first target nucleic acid sequence.
10. A method of introducing a nucleic acid break in a first target nucleic acid, comprising the steps of:
   a. designing a guide-RNA (crRNA) capable of recognising a second target nucleic acid comprising a protospacer adjacent motif (PAM);
   b. contacting the crRNA of step a. with a mutant Cas12j endonuclease or orthologue thereof, wherein the mutant Cas12j endonuclease or orthologue thereof is according to any one of items 1 to 4, or encoded by a polynucleotide or a vector according to any one of items 5 to 6, thereby obtaining a crRNA-Cas12j complex capable of binding to said second target nucleic acid, and
   c. contacting the crRNA and the mutant Cas12j endonuclease with said first target nucleic acid,
thereby introducing one or more nucleic acid breaks in the first target nucleic acid.
11. An in vitro method of introducing a site-specific, double-stranded break at a second target nucleic acid in a mammalian cell, the method comprising introducing into the mammalian cell a crRNA-Cas12j complex, wherein the Cas12j is a mutant Cas12j endonuclease or orthologue according to any one of items 1 to 4, and wherein the crRNA is specific for the second target nucleic acid.
12. A method for detection of a second target nucleic acid in a sample, the method comprising:
   a. Providing a crRNA-Cas12j complex, wherein the Cas12j is a mutant Cas12j endonuclease or orthologue thereof according to any one of items 1 to 4, and wherein the crRNA is specific for the second target nucleic acid;
   b. Providing a labelled ssDNA, wherein the ssDNA is labelled with at least one set of interactive labels comprising at least one dye and at least one quencher;
   c. Contacting the crRNA-Cas12j complex and the ssDNA with the sample, wherein the sample comprises at least one second target nucleic acid; and
   d. Detecting cleavage of the ssDNA by detecting a fluorescent signal from the fluorophore; and
   e. Optionally, determining the level and/or concentration of the second target nucleic acid, wherein the level and/or concentration of the second target nucleic acid is correlated to the cleaved ssDNA,
thereby detecting the presence of the second target nucleic acid in the sample, wherein step c. optionally comprises activation of the crRNA-Cas12j complex.
13. A method for detection and optionally quantification of a second target nucleic acid, such as a nucleic acid fragment of a viral genome, a microbial genome, a gene of a pathogen, or a nucleic acid sequence associated with a human disease, in a sample, the method comprising:
   a. Providing a crRNA-Cas12j complex, wherein the Cas12j is a mutant Cas12j endonuclease or orthologue thereof according to any one of items 1 to 4, wherein
      i. the mutant Cas12j has an abrogated endonuclease activity;
      ii. the mutant Cas12j comprises a detectable protein label; and
      iii, the crRNA is specific for the second target nucleic acid;
   b. Contacting the crRNA-Cas12j complex with the sample, wherein the sample comprises at least one second target nucleic acid; and
   c. Detecting and optionally quantifying the presence of the second target nucleic acid by detecting the protein label, such as a fluorescent signal.
14. An in vitro method for diagnosis of a disease in a subject, the method comprising:
   a. Providing a crRNA-Cas12j complex, wherein the Cas12j is a mutant Cas12j endonuclease or orthologue thereof according to any one of items 1 to 4, and wherein the crRNA is specific for a second target nucleic acid;
   b. Providing a labelled ssDNA, wherein the ssDNA is labelled with at least one set of interactive labels comprising at least one dye and at least one quencher;
   c. Providing a sample from the subject, wherein said sample comprises or is suspected of comprising the second target nucleic acid; and
   d. Determining the level and/or concentration of the second target nucleic acid as defined in any one of the preceding items,
wherein the second target nucleic acid is a nucleic acid fragment that correlates with the disease, such as wherein the second target nucleic acid is a biomarker of the disease,
thereby diagnosing a disease in a subject.
15. An in vitro method for diagnosis of an infectious disease in a subject, the method comprising:
   a. Providing a crRNA-Cas12j complex, wherein the Cas12j is a mutant Cas12j endonuclease or orthologue thereof according to any one of items 1 to 4, and wherein the crRNA is specific for a second target nucleic acid;
   b. Providing a labelled ssDNA, wherein the ssDNA is labelled with at least one set of interactive labels comprising at least one dye and at least one quencher;
   c. Providing a sample from the subject, wherein said sample comprises or is suspected of comprising the second target nucleic acid; and
   d. Determining the level and/or concentration of the second target nucleic acid as defined in any one of the preceding items,
wherein the second target nucleic acid is a nucleic acid of the genome of an infectious agent causing the disease or a fragment thereof, thereby diagnosing an infectious disease in a subject.

1. A mutant Cas12j comprising a polypeptide sequence having at least 95% sequence identity to:
i) the sequence corresponding to residues 1 to 20, 36 to 97, 104 to 119, 151 to 179, 204 to 379, 396 to 619, 651 to 679, and 701 to 726 of SEQ ID NO: 3, wherein said polypeptide sequence further comprises:
a. at least one amino acid mutation in a first region of the NPID domain corresponding to residues 21 to 35 of SEQ ID NO: 3, wherein each mutation independently is an amino acid substitution, insertion or deletion; and/or
b. at least one amino acid mutation in a first region of the TPID domain corresponding to residues 98 to 103 of SEQ ID NO: 3, wherein each mutation independently is an amino acid substitution, insertion or deletion; and/or
c. at least one amino acid mutation in a second region of the TPID domain corresponding to residues 120 to 150 of SEQ ID NO: 3, wherein each mutation independently is an amino acid substitution, insertion or deletion; and/or
d. at least one amino acid mutation in a third region of the TPID domain or in a first region of the RBD domain corresponding to residues 180 to 203 of SEQ ID NO: 3, wherein each mutation independently is an amino acid substitution, insertion or deletion; and/or
e. at least one amino acid mutation in a second region of the RBD domain or in a first region of the RuvC-I domain corresponding to residues 380 to 395 of SEQ ID NO: 3, wherein each mutation independently is an amino acid substitution, insertion or deletion; and/or
f. at least one amino acid mutation in a first region of the RuvC-II domain corresponding to residues 620 to 650 of SEQ ID NO: 3, wherein each mutation independently is an amino acid substitution, insertion or deletion; and/or
g. at least one amino acid mutation in a second region of the RuvC-II domain corresponding to residues 680 to 700 of SEQ ID NO: 3, wherein each mutation independently is an amino acid substitution, insertion or deletion; and/or
h. at least one amino acid mutation in a third region of the RuvC-II domain corresponding to residues 726 to 766 of SEQ ID NO: 3, wherein each mutation independently is an amino acid substitution, insertion or deletion; and/or
ii) SEQ ID NO: 3, wherein said polypeptide sequence comprises at least one amino acid substitution in a position selected from the positions corresponding to residues 26, 30, 54, 55, 123, 197, 355, 360, 413, 618, 625, 626, 630, 643, 673, 675, 676, 680, 683, 691, 698, 701 and 708 of SEQ ID NO: 3.

2. (canceled)

3. The mutant Cas12j or orthologue thereof according to claim 1, wherein said mutant Cas12j comprises a polypeptide sequence having at least 95% sequence identity to the sequence corresponding to residues 1 to 726 of SEQ ID NO: 3, wherein said polypeptide sequence further comprises a C-terminal deletion of the sequence corresponding to residues 727 to 766 of SEQ ID NO: 3; and/or wherein the mutant Cas12j comprises or consists of a polypeptide sequence having at least 95% sequence identity to SEQ ID NO: 31.

4. (canceled)

5. The mutant Cas12j or orthologue thereof according to claim 1, wherein the at least one amino acid substitution is a substitution of an amino acid having a charged side chain to an amino acid having an uncharged side chain.

6. The mutant Cas12j or orthologue thereof according to claim 1, wherein the at least one amino acid substitution is a) a substitution of an amino acid having a charged side chain to an amino acid residue having a non-polar side chain; and/or b) a substitution of an amino acid having a charged side chain to an amino acid residue having a non-polar side chain; and/or c) a substitution of an amino acid having a charged side chain to a glycine, alanine, valine, leucine, isoleucine, serine or threonine; and/or d) a substitution of an amino acid having a charged side chain to a glycine; and/or e) a substitution of an amino acid to an alanine.

7-10. (canceled)

11. The mutant Cas12j or orthologue thereof according to claim 1, wherein the at least one amino acid substitution is in the NPID domain; and/or wherein the at least one amino acid substitution is in the TPID domain; and/or wherein the at least one amino acid substitution is in the RBD domain; and/or wherein the at least one amino acid substitution is in the RuvC-I domain; and/or wherein the at least one amino acid substitution is in the RuvC-II domain.

12-16. (canceled)

17. The mutant Cas12j or orthologue thereof according to claim 1, wherein the mutant Cas12j is a nicking endonuclease.

18. The mutant Cas12j or orthologue thereof according to claim 1, comprising a substitution at one or more positions selected independently from the group consisting of: K26; K30; F54; K55; Q123; Q197; L355; T360; D413; E618; K625; F626; G630; R643; P673; W675; T676; C680; C683; R691; R691; C698; C701; and D708 of SEQ ID NO: 3 or SEQ ID NO: 31.

19-41. (canceled)

42. The mutant Cas12j or orthologue thereof according to claim 1, wherein the mutant Cas12j is a mutant of a Cas12j selected from the group consisting of CasΦ-1 (SEQ ID NO: 1), CasΦ-2 (SEQ ID NO: 2), CasΦ-3 (SEQ ID NO: 3), CasΦ-4 (SEQ ID NO: 4), CasΦ-5 (SEQ ID NO: 5), CasΦ-6 (SEQ ID NO: 6), CasΦ-7 (SEQ ID NO: 7), CasΦ-8 (SEQ ID NO: 8), CasΦ-9 (SEQ ID NO: 9), and CasΦ-10 (SEQ ID NO: 10).

43. (canceled)

44. The mutant Cas12j or orthologue thereof according to claim 1, wherein the mutant Cas12j has one or more altered activities compared to the wild type Cas12j, said activity being selected from the group consisting of double-stranded cleavage of a target nucleic acid sequence, single-stranded cleavage of a target nucleic acid sequence and target nucleic acid recognition.

45-54. (canceled)

55. A polynucleotide encoding the mutant Cas12j endonuclease or orthologue thereof according to claim 1.

56. The polynucleotide according to claim 0, wherein the mutant Cas12j is encoded by a polynucleotide comprising or consisting of a nucleic acid sequence with at least 80% sequence identity to a nucleic acid sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 32 and SEQ ID NO: 33.

57-58. (canceled)

59. A recombinant vector comprising nucleic acid sequence encoding a mutant Cas12j or orthologue thereof according to claim 1

60-64. (canceled)

65. A cell capable of expressing the mutant Cas12j or orthologue thereof according to claim 1 a polynucleotide encoding the mutant Cas12j or orthologue thereof according to claim 1 a recombinant vector comprising a nucleic acid sequence encoding a mutant Cas12j or orthologue thereof according to claim 1.

66. A system for expression of a crRNA-Cas12j complex comprising
a. a polynucleotide encoding the mutant Cas12j or orthologue thereof according to claim 1 a recombinant vector comprising a polynucleotide encoding a mutant Cas12j or orthologue thereof according to claim 1;
b. a polynucleotide or a recombinant vector comprising a polynucleotide encoding a guide RNA (crRNA), optionally operably linked to a promoter.

67-68. (canceled)

69. Use of a crRNA-Cas12j complex in a method for introducing a nucleic acid break in a first target nucleic acid, wherein:
a. a mutant Cas12j or orthologue thereof is contacted with a guide RNA (crRNA), thereby obtaining a crRNA-Cas12j complex capable of recognizing a second target nucleic acid, the second target nucleic acid comprising a protospacer adjacent motif (PAM), and wherein the Cas12j or orthologue thereof is according to claim 1
b. the crRNA-Cas12j complex is contacted with the first target nucleic acid; whereby a nucleic acid break is made in the first target nucleic acid sequence.

70-79. (canceled)

80. A method of introducing a nucleic acid break in a first target nucleic acid, comprising the steps of:
a. designing a guide-RNA (crRNA) capable of recognising a second target nucleic acid comprising a protospacer adjacent motif (PAM);
b. contacting the crRNA of step a. with a mutant Cas12j or orthologue thereof, wherein the mutant Cas12j or orthologue thereof is according to claim 1, or wherein the mutant Cas12j or orthologue thereof according to claim 1 is encoded by a polynucleotide or a vector hereby obtaining a crRNA-Cas12j complex capable of binding to said second target nucleic acid, and
c. contacting the crRNA and the mutant Cas12j with said first target nucleic acid, thereby introducing one or more nucleic acid breaks in the first target nucleic acid.

81-85. (canceled)

86. An in vitro method of introducing a site-specific, double-stranded break at a second target nucleic acid in a mammalian cell, the method comprising introducing into the mammalian cell a crRNA-Cas12j complex, wherein the Cas12j is a mutant Cas12j or orthologue according to claim 1 and wherein the crRNA is specific for the second target nucleic acid.

87. A method for detection of a second target nucleic acid in a sample, the method comprising:
a. Providing a crRNA-Cas12j complex, wherein the Cas12j is a mutant Cas12j or orthologue thereof according to claim 1 and wherein the crRNA is specific for the second target nucleic acid;
b. Providing a labelled ssDNA, wherein the ssDNA is labelled with at least one set of interactive labels comprising at least one dye and at least one quencher;
c. Contacting the crRNA-Cas12j complex and the ssDNA with the sample, wherein the sample comprises at least one second target nucleic acid; and
d. Detecting cleavage of the ssDNA by detecting a fluorescent signal from the fluorophore,
thereby detecting the presence of the second target nucleic acid in the sample, wherein step c. optionally comprises activation of the crRNA-Cas12j complex.

88-93. (canceled)

94. A method for detection and optionally quantification of a second target nucleic acid in a sample, the method comprising:
a. Providing a crRNA-Cas12j complex, wherein the Cas12j is a mutant Cas12j or orthologue thereof according to claim 1 wherein
i. the mutant Cas12j has an abrogated activity;
ii. the mutant Cas12j comprises a detectable protein label; and
iii, the crRNA is specific for the second target nucleic acid;
b. Contacting the crRNA-Cas12j complex with the sample, wherein the sample comprises at least one second target nucleic acid; and
c. Detecting and optionally quantifying the presence of the second target nucleic acid by detecting the protein label, such as a fluorescent signal.

95-97. (canceled)

98. An in vitro method for diagnosis of a disease in a subject, the method comprising:
a. Providing a crRNA-Cas12j complex, wherein the Cas12j is a mutant Cas12j or orthologue thereof according to claim 1 and wherein the crRNA is specific for a second target nucleic acid;
b. Providing a labelled ssDNA, wherein the ssDNA is labelled with at least one set of interactive labels comprising at least one dye and at least one quencher;
c. Providing a sample from the subject, wherein said sample comprises or is suspected of comprising the second target nucleic acid; and
d. Determining the level and/or concentration of the second target nucleic acid as defined in any one of the preceding claims,
wherein the second target nucleic acid is a nucleic acid fragment that correlates with the disease, such as wherein the second target nucleic acid is a biomarker of the disease, thereby diagnosing a disease in a subject.

99. (canceled)

100. An in vitro method for diagnosis of an infectious disease in a subject, the method comprising:
e. Providing a crRNA-Cas12j complex, wherein the Cas12j is a mutant Cas12j or orthologue thereof according to claim 1 and wherein the crRNA is specific for a second target nucleic acid;
f. Providing a labelled ssDNA, wherein the ssDNA is labelled with at least one set of interactive labels comprising at least one dye and at least one quencher;
g. Providing a sample from the subject, wherein said sample comprises or is suspected of comprising the second target nucleic acid; and
h. Determining the level and/or concentration of the second target nucleic acid as defined in any one of the preceding claims,
wherein the second target nucleic acid is a nucleic acid of the genome of an infectious agent causing the disease or a fragment thereof,
thereby diagnosing an infectious disease in a subject.

101-107. (canceled)
